SEQUENCE LISTING (65 sequences)

SEQ ID NO: 1 - DNA, 40 bp, Artificial Sequence
  Description of Artificial Sequence Primer Sequence
atatggatcccaatgttgggtctctttctcaattagttgc

SEQ ID NO: 2 - DNA, 33 bp, Artificial Sequence
  Description of Artificial Sequence Primer Sequence
atatggatccgattgatgtgggaggccagggtt

SEQ ID NO: 3 - DNA, 40 bp, Artificial Sequence
  Description of Artificial Sequence Primer Sequence
atatggatccaacaaaagcaaaacactgtttcattagcgg

SEQ ID NO: 4 - DNA, 38 bp, Artificial Sequence
  Description of Artificial Sequence Primer Sequence
atatggatcccctagactggagttaatgacggtcgtga

SEQ ID NO: 5 - DNA, 45 bp, Artificial Sequence
  Description of Artificial Sequence Primer Sequence
atatggatccgtatatgagtacactgtagctcttttcagacacac

SEQ ID NO: 6 - DNA, 31 bp, Artificial Sequence
  Description of Artificial Sequence Primer Sequence
atatggatcccccagagcacggctaaggggt

SEQ ID NO: 7 - DNA, 39 bp, Artificial Sequence
  Description of Artificial Sequence Primer Sequence
atatggatccttcatgagacattataccatctcacatct

SEQ ID NO: 8 - DNA, 37 bp, Artificial Sequence
  Description of Artificial Sequence Primer Sequence
atatggatccggctcagaagaaagacagtgtacggtg

SEQ ID NO: 9 - DNA, 35 bp, Artificial Sequence
  Description of Artificial Sequence Primer Sequence
atatggatccgacaggcgatgggataagacacctg

SEQ ID NO: 10 - DNA, 31 bp, Artificial Sequence
  Description of Artificial Sequence Primer Sequence
atatggatccagggaggcagtgtgggctgaa

SEQ ID NO: 11 - DNA, 32 bp, Artificial Sequence
  Description of Artificial Sequence Primer Sequence
atatggatcccatgggacctggccaggtttca

SEQ ID NO: 12 - DNA, 32 bp, Artificial Sequence
  Description of Artificial Sequence Primer Sequence
atatggatccggctggcagcagaaatcgggaa

SEQ ID NO: 13 - DNA, 30 bp, Artificial Sequence
  Description of Artificial Sequence Primer Sequence
atatggtaccgaaggccagagtgcctgtcc

SEQ ID NO: 14 - DNA, 33 bp, Artificial Sequence
  Description of Artificial Sequence Primer Sequence
atatgtcgacaaatcgctgtggtttgaatgcca

SEQ ID NO: 15 - DNA, 34 bp, Artificial Sequence
  Description of Artificial Sequence Primer Sequence
atatgtcgacgtgtgcactaccatgtccagcatg

SEQ ID NO: 16 - DNA, 33 bp, Artificial Sequence
  Description of Artificial Sequence Primer Sequence
atatgtcgacagggagaaccaagctacccagga

SEQ ID NO: 17 - DNA, 42 bp, Artificial Sequence
  Description of Artificial Sequence Primer Sequence
atatgtcgacagaaagattacccacaaatcaaaaccagggct

SEQ ID NO: 18 - DNA, 37 bp, Artificial Sequence
  Description of Artificial Sequence Primer Sequence
atatgtcgacggacagcttaaagagacaggctgagat

SEQ ID NO: 19 - DNA, 33 bp, Artificial Sequence
  Description of Artificial Sequence Primer Sequence
atatgtcgaccatcctgtcctgagcaagccctt

SEQ ID NO: 20 - DNA, 31 bp, Artificial Sequence
  Description of Artificial Sequence Primer Sequence
atatgtcgactgagggattcatccatgcaga

SEQ ID NO: 21 - DNA, 45 bp, Artificial Sequence
  Description of Artificial Sequence Primer Sequence
atatgtcgacgtttgtttgttttgttttttcgagacagggtttct

SEQ ID NO: 22 - DNA, 36 bp, Artificial Sequence
  Description of Artificial Sequence Primer Sequence
atatctcgagtctgcaatcccagtgtgtcaggagac

SEQ ID NO: 23 - DNA, 39 bp, Artificial Sequence
  Description of Artificial Sequence Primer Sequence
atatgtcgacgtctagctacctccatgaaactctgcacc

SEQ ID NO: 24 - DNA, 46 bp, Artificial Sequence
  Description of Artificial Sequence Primer Sequence
atatgtcgacattctccctccatatatacatctgttctatcgacag

SEQ ID NO: 25 - DNA, 31 bp, Artificial Sequence
  Description of Artificial Sequence Primer Sequence
atatgtcgaccgaccaggaggagggggtgtt

SEQ ID NO: 26 - DNA, 28 bp, Artificial Sequence
  Description of Artificial Sequence Primer Sequence
atatgagctctcccactccctgtggccg

SEQ ID NO: 27 - DNA, 20 bp, Artificial Sequence
  Description of Artificial Sequence Synthetic sgRNA
gtgaactgaactccccaccg

SEQ ID NO: 28 - DNA, 20 bp, Artificial Sequence
  Description of Artificial Sequence Synthetic sgRNA
gcgctgagatatatgaatgc

SEQ ID NO: 29 - DNA, 20 bp, Artificial Sequence
  Description of Artificial Sequence Synthetic sgRNA
gggcggggcggttcctgatt

SEQ ID NO: 30 - DNA, 20 bp, Artificial Sequence
  Description of Artificial Sequence Synthetic sgRNA
gcagcagggtcagcgtgaac

SEQ ID NO: 31 - DNA, 20 bp, Artificial Sequence
  Description of Artificial Sequence Synthetic sgRNA
tgcttaggcacggtagtcat

SEQ ID NO: 32 - DNA, 20 bp, Artificial Sequence
  Description of Artificial Sequence Synthetic sgRNA
tctggtctggtcactacaaa

SEQ ID NO: 33 - DNA, 20 bp, Artificial Sequence
  Description of Artificial Sequence Synthetic sgRNA
gtgatctaacacacccccct

SEQ ID NO: 34 - DNA, 20 bp, Artificial Sequence
  Description of Artificial Sequence Synthetic sgRNA
gggtttggggctagtgtaat

SEQ ID NO: 35 - DNA, 20 bp, Artificial Sequence
  Description of Artificial Sequence Synthetic sgRNA
ggggtttgacctgagccatc

SEQ ID NO: 36 - DNA, 20 bp, Artificial Sequence
  Description of Artificial Sequence Synthetic sgRNA
gagcttttggtgtcttgacc

SEQ ID NO: 37 - DNA, 20 bp, Artificial Sequence
  Description of Artificial Sequence Synthetic sgRNA
ggaggggttaactaacccac

SEQ ID NO: 38 - DNA, 20 bp, Artificial Sequence
  Description of Artificial Sequence Synthetic sgRNA
ggatcaataactacttggct

SEQ ID NO: 39 - DNA, 20 bp, Artificial Sequence
  Description of Artificial Sequence Synthetic sgRNA
tagccatctcccagtcaagc

SEQ ID NO: 40 - DNA, 20 bp, Artificial Sequence
  Description of Artificial Sequence Synthetic sgRNA
atggacccttactcccaaat

SEQ ID NO: 41 - DNA, 20 bp, Artificial Sequence
  Description of Artificial Sequence Synthetic sgRNA
gaggtgaagggtcccaatcg

SEQ ID NO: 42 - DNA, 20 bp, Artificial Sequence
  Description of Artificial Sequence Synthetic sgRNA
ctgcgttttaagccttataa

SEQ ID NO: 43 - DNA, 20 bp, Artificial Sequence
  Description of Artificial Sequence Synthetic sgRNA
gtgaactgaactccccaccg

SEQ ID NO: 44 - DNA, 20 bp, Artificial Sequence
  Description of Artificial Sequence Synthetic sgRNA
gcgctgagatatatgaatgc

SEQ ID NO: 45 - DNA, 20 bp, Artificial Sequence
  Description of Artificial Sequence Synthetic sgRNA
gggcggggcggttcctgatt

SEQ ID NO: 46 - DNA, 20 bp, Artificial Sequence
  Description of Artificial Sequence Synthetic sgRNA
gcagcagggtcagcgtgaac

SEQ ID NO: 47 - DNA, 20 bp, Artificial Sequence
  Description of Artificial Sequence Synthetic sgRNA
ggcttaggcacggtagtcat

SEQ ID NO: 48 - DNA, 20 bp, Artificial Sequence
  Description of Artificial Sequence Synthetic sgRNA
gctggtctggtcactacaaa

SEQ ID NO: 49 - DNA, 20 bp, Artificial Sequence
  Description of Artificial Sequence Synthetic sgRNA
gtgatctaacacacccccct

SEQ ID NO: 50 - DNA, 20 bp, Artificial Sequence
  Description of Artificial Sequence Synthetic sgRNA
gggtttggggctagtgtaat

SEQ ID NO: 51 - DNA, 20 bp, Artificial Sequence
  Description of Artificial Sequence Synthetic sgRNA
ggggtttgacctgagccatc

SEQ ID NO: 52 - DNA, 20 bp, Artificial Sequence
  Description of Artificial Sequence Synthetic sgRNA
gagcttttggtgtcttgacc

SEQ ID NO: 53 - DNA, 20 bp, Artificial Sequence
  Description of Artificial Sequence Synthetic sgRNA
ggaggggttaactaacccac

SEQ ID NO: 54 - DNA, 20 bp, Artificial Sequence
  Description of Artificial Sequence Synthetic sgRNA
ggatcaataactacttggct

SEQ ID NO: 55 - DNA, 20 bp, Artificial Sequence
  Description of Artificial Sequence Synthetic sgRNA
gagccatctcccagtcaagc

SEQ ID NO: 56 - DNA, 20 bp, Artificial Sequence
  Description of Artificial Sequence Synthetic sgRNA
gtggacccttactcccaaat

SEQ ID NO: 57 - DNA, 20 bp, Artificial Sequence
  Description of Artificial Sequence Synthetic sgRNA
gaggtgaagggtcccaatcg

SEQ ID NO: 58 - DNA, 20 bp, Artificial Sequence
  Description of Artificial Sequence Synthetic sgRNA
gtgcgttttaagccttataa

SEQ ID NO: 59 - DNA, 118 bp, Artificial Sequence
  Description of Artificial Sequence Synthetic sgRNA
taatacgactcactataggtgaactgaactccccaccggttttagagctagaaatagcaagttaaaataaggctagtccgttatcaacttgaaaaagtggcaccgagtcggtgctttt

SEQ ID NO: 60 - DNA, 20 bp, Artificial Sequence
  Description of Artificial Sequence Primer Sequence
gcatctctgctccccacttt

SEQ ID NO: 61 - DNA, 20 bp, Artificial Sequence
  Description of Artificial Sequence Primer Sequence
ggctcccagtgtgacctttt

SEQ ID NO: 62 - DNA, 20 bp, Artificial Sequence
  Description of Artificial Sequence Primer Sequence
ctgaggctccttatcgggga

SEQ ID NO: 63 - DNA, 20 bp, Artificial Sequence
  Description of Artificial Sequence Primer Sequence
cagtaagccaccttgagcca

SEQ ID NO: 64 - DNA, 20 bp, Artificial Sequence
  Description of Artificial Sequence Primer Sequence
cctgaacgcctgagctaaca

SEQ ID NO: 65 - DNA, 20 bp, Artificial Sequence
  Description of Artificial Sequence Primer Sequence
ctgaatgcccaaaacgcacc

(19) United States
(12) Patent Application Publication
(10) Pub. No.: US 2018/0251533 A1
(43) Pub. Date: Sep. 6, 2018

(54) METHODS AND COMPOSITIONS FOR MODULATING MONOCYTE POPULATIONS AND RELATED USES THEREOF

(71) Applicant: La Jolla Institute for Allergy and Immunology, La Jolla, CA (US)

(72) Inventors: Catherine HEDRICK, La Jolla, CA (US); Graham THOMAS, La Jolla, CA (US)

(73) Assignee: La Jolla Institute for Allergy and Immunology, La Jolla, CA (US)

(21) Appl. No.: 15/759,017

(22) PCT Filed: Sep. 12, 2016
(86) PCT No.: PCT/US2016/051308

§ 371 Date: Mar. 9, 2018

Related U.S. Application Data
(60) Provisional application No. 62/216,976, filed on Sep. 10, 2015.

(51) Int. Cl. C07K 16/18; C12N 15/113; C12Q 1/6883

(52) U.S. Cl. CPC C07K 16/18; C07K 2317/76; C12Q 1/6883; C12N 15/113

(57) ABSTRACT

Method and compositions for modulating specific populations of monocytes or macrophages are disclosed. Methods include, in certain embodiments, modulating expression or activity of Nr4a1 (Nur77). Compositions disclosed herein include agonistic and antagonistic agents that modulate expression or activity of Nur77 and uses thereof. In various embodiments, methods of treating certain disorders and diseases related to aberrant monocyte or macrophage development are provided. In further various embodiments, methods of identifying agents that modulate specific populations of monocytes or macrophages, and agents that modulate Nur77 activity and/or expression are provided.

### RELATED APPLICATIONS

This patent application claims the benefit of, and priority to, U.S. Provisional Patent Application No. 62/216,976 filed on Sep. 10, 2015. The entire content of the foregoing application is incorporated herein by reference, including all text, tables and drawings.

### GOVERNMENT SUPPORT

This invention was made, in part, with government support under grant number RO1 HL134236 awarded by NIH. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Sep. 9, 2016, is named LIAI0448513_ST25.txt and is 15 KB (15,022 bytes) in size.

### INTRODUCTION

Monocytes are the main immune cell type that infiltrate atherosclerotic plaques, and a substantial body of evidence implicates monocytes as key players in the early stages of atherosclerosis. Mice have two major monocyte populations that can be found in the bone marrow (BM) and circulating in the blood. Both of these populations express the myeloid-defining markers CD11b and Csf1r (CD115), which can be used to identify these cells by flow cytometry. The two major monocyte populations can be separated based on the expression of the cell surface antigen Ly6C.

Ly6C^{hi} monocytes are the short-lived precursors of inflammatory macrophages and are key drivers of atherosclerosis¹. Ly6C^{hi} monocytes are recruited to atherosclerotic lesions in a Ccr2 and Ccr5-dependent manner, and preventing recruitment almost completely abrogates atherosclerosis in mice fed a high diet². Less in known about Ly6C^{low} monocyte function however they do infiltrate atherosclerotic lesions², and circumstantial evidence suggests that they protect against atherosclerosis, and generally act in wound repair^{3,4}. Ly6C^{low} monocytes display a characteristic patrolling behaviour on the vascular endothelium⁵ and help to remove damaged endothelial cells⁶.

Ly6C^{low} monocyte development requires the transcription factor Nr4a1 (Nur77; Nuclear Receptor Subfamily 4 Group A Member 1; e.g., UniProtKB: P22736) as Nr4a1^{-/-} mice completely lack Ly6C^{low} monocytes⁷. Nr4a1 is highly expressed and its effects are cell-intrinsic. Hence Nr4a1 is a lineage-determining transcription factor for Ly6C^{low} monocytes. In two independent models of atherosclerosis (Apoe^{-/-} and Ldlr^{-/-}), Nr4a1^{-/-} mice develop worse disease³ indicating a restorative function for Ly6C^{low} monocytes in atherosclerosis. This interpretation is confounded by the fact that Nr4a1^{-/-} macrophages are more inflammatory, a processes that adversely affects atherosclerosis³.

Evidence suggests that the human CD14⁺CD16⁻ monocytes are equivalent to the mouse Ly6C^{hi} population and that CD14^{dim}CD16⁺ monocytes are similar to Ly6C^{low} monocytes^{8,9}. CD14^{dim}CD16⁺ monocytes also have high Nr4a1 expression suggesting conserved function and raising the possibility that modulating Nr4a1 may allow manipulation of this subset in humans.

### SUMMARY

Disclosed herein is the identification of transcription factors that regulate Nr4a1 during Ly6C^{low} or CD14^{dim}CD16⁺ monocyte development. Modulation of such transcription factors allows for treatment of diseases as set forth herein.

In a first aspect, there is provided a method of treating an autoimmune disease in a subject in need thereof, the method including administering to the subject an effective amount of a PTPRA (Protein Tyrosine Phosphatase, Receptor Type A) antagonist.

In another aspect, there is provided a method of decreasing inflammation in a synovium of a subject in need thereof, the method including administering to the subject an effective amount of a PTPRA antagonist.

In another aspect, there is provided a method of decreasing expression of PTPRA in a fibroblast-like synoviocyte, the method including contacting said fibroblast-like synoviocyte with an effective amount of a PTPRA antagonist.

In another aspect, there is provided a method of decreasing TNF activity, IL-1 activity and/or PDGF activity in a fibroblast-like synoviocyte, the method including contacting the fibroblast-like synoviocyte with an effective amount of a PTPRA antagonist.

In another aspect, there is provided a method of decreasing invasiveness or migration of a fibroblast-like synoviocyte, the method including contacting the fibroblast-like synoviocyte with an effective amount of a PTPRA antagonist.

In another aspect, there is provided a pharmaceutical composition including a PTPRA antagonist and a pharmaceutically acceptable excipient.

In some aspects, there is provided a method of modulating CD14^{dim}CD16⁺ (CD115⁺CD11b⁺GR1⁻ (Ly6C⁻)) monocytes or macrophages. In some embodiments, methods of modulating CD14^{dim}CD16⁺ (CD115⁺CD11b⁺GR1⁻ (Ly6C⁻)) monocytes or macrophages comprises increasing or decreasing the amounts of CD14^{dim}CD16⁺ (CD115⁺CD11b⁺GR1⁻ (Ly6C⁻)) monocytes or macrophages in a subject (e.g., mammal or human). As described herein, the expression or activity of Nr4a1 (Nur77) can directly effect the activity and/or amounts of CD14dimCD16+(CD115+CD11b+GR1− (Ly6C−)) monocyte/macrophage populations in a subject. Thus, in certain embodiments, a method of modulating CD14^{dim}CD16⁺ (CD115⁺CD11b⁺GR1⁻ (Ly6C⁻)) monocytes or macrophages comprises modulating expression or activity of a transcription factor that regulates expression of Nr4a1 (Nur77). In certain aspects, a method of stimulating, promoting, increasing or inducing CD14^{dim}CD16⁺ (CD115⁺CD11b⁺GR1⁻ (Ly6C⁻)) monocyte or macrophage cell production, development, survival, proliferation, differentiation or activity, comprises modulating expression or activity of a transcription factor that regulates expression of Nr4a1 (Nur77).

Certain transcription factors are disclosed herein that bind to a 5′ upstream regulatory DNA sequence in the 5′ untranslated region (UTR) of the Nr4a1 gene, the cis-regulatory promoter region of the Nr4a1 gene, or in any other trans-acting Nr4a1-associated enhancer sequence, such as those identified herein, where such transcription factors can modulate Nr4a1 gene expression. Accordingly, disclosed herein, in certain embodiments, are transcription factors that bind to specific regulatory DNA sequences located in the 5′ UTR of the Nr4a1 gene, such as enhancer region 2 (Nr4a1se_2) in Ly6C− monocyte or upstream progenitor cell types thereof, or a homologous region in CD14^{dim}CD16⁺ (CD115⁺CD11b⁺GR1⁻ (Ly6C⁻)) monocytes or upstream progenitor cell types thereof. In some embodiments, the transcription factors are Klf2 and Klf4.

In some aspects, there are provided methods of inhibiting growth of a hyperproliferative cell, tumor cell, cancer cell, neoplastic cell, metastatic cell or tumor, wherein the method comprises administering an agonist or antagonist to a subject. In certain embodiments, an agonist or antagonist is an agonist or antagonist of a transcription factor identified herein. In certain embodiments, an agonist or antagonist is an agonist or antagonist of a Nur77 transcription or expression. In certain embodiments, an agonist or antagonist is an antibody, a small molecule, a peptide, an inhibitory nucleic acid, an allosteric inhibitor or a ligand mimetic. In some embodiments, an antagonist or antagonist is an antibody, or binding fragment thereof, that specifically binds to a Klf2 or Klf4 polypeptide. In some embodiments, an antagonist is an inhibitory nucleic acid that targets or binds to an mRNA directing the expression of a Klf2 or Klf4 polypeptide.

In some aspects, a method comprises decreasing, reducing, inhibiting, suppressing, limiting or controlling an undesirable or aberrant immune response, immune disorder, inflammatory response, inflammation autoimmune response, autoimmune disease, adverse cardiovascular event or cardiovascular disease in the subject. In certain embodiments, the method comprises administering an agent, agonist, or antagonist to a subject to treat or prevent an undesirable or aberrant immune response, immune disorder, inflammatory response, inflammation autoimmune response, autoimmune disease, adverse cardiovascular event or cardiovascular disease. In some embodiments, the method comprises treating cancer in a subject.

In certain aspects, there is provided a pharmaceutical composition comprising an agonist or antagonist of a transcription factor that regulates expression of Nr4a1 (Nur77) for treatment of an aberrant immune response, immune disorder, inflammatory response, inflammation, an autoimmune response, disorder or disease, cancer or an adverse cardiovascular event or cardiovascular disease in a subject. In some embodiments, the transcription factor binds to the DNA sequence in the region Nr4a1se_2 in Ly6C− monocyte or upstream progenitor cell types thereof or a homologous region in CD14^{dim}CD16⁺ (CD115⁺CD11b⁺GR1⁻ (Ly6C⁻)) monocytes or upstream progenitor cell types thereof. In certain embodiments, the transcription factor is Klf2 or Klf4. In certain embodiments, the agonist or antagonist is an antibody, a small molecule, a peptide, an inhibitory nucleic acid, an allosteric inhibitor or a ligand mimetic.

In some aspects, provided herein are methods of identifying an agent that modulates, increases or decreases CD14^{dim}CD16⁺ (CD115⁺CD11b⁺GR1⁻ (Ly6C⁻)) monocyte or macrophage amounts or activity. A representative method comprises: (a) contacting an experimental agent with an Nr4a1 promoter region in the presence of Klf2, Klf4 and/or Nf-κB, wherein the Nr4a1 promoter region is operably linked to a reporter nucleic acid; (b) determining an amount of the reporter nucleic acid, or a transcribed or translated product thereof; (c) comparing the amount determined in (b) to a control amount of reporter nucleic acid, or a transcribed or translated product thereof, wherein a difference between the amount determined in (b) and the control amount indicates the experimental agent is an agent that modulates, increases or decreases CD14^{dim}CD16⁺ (CD115⁺CD11b⁺GR1⁻ (Ly6C⁻)) monocyte or macrophage amounts or activity. In some embodiments, a experimental agent is an antibody or inhibitory nucleic acid. In some embodiments, a experimental agent is an antibody that specifically binds to Klf2 or Klf4.

In some embodiments, presented herein is a vertebrate, (e.g., a mammal, e.g., a rodent; e.g., a rat, e.g., a mouse, or the like) comprising deficient, disrupted or modified Nr4a1se_2 sequence. A vertebrate deficient in an Nr4a1se_2 sequence can be homozygous or heterozygous for a deleted, modified or disrupted Nr4a1se_2A sequence. In some embodiments, presented herein is method of using a vertebrate, (e.g., a mammal, e.g., a rodent; e.g., a rat, e.g., a mouse, or the like) comprising a deficient, disrupted or modified Nr4a1se_2 sequence to identify CD14dimCD16⁺ (CD115+CD11b+GR1−(Ly6C⁻)) monocyte functions.

### BRIEF DESCRIPTION OF THE DRAWINGS

FIG. 1. shows a UCSC genome browser screenshot showing, from top to bottom, PU.1 peaks in Ly6C^{low} monocytes, overlays of H3K27ac and H3K4me2 respectively with Ly6C^{hi} monocytes in Red and Ly6C^{low} monocytes in blue. The lower tracks show our enhancer candidate predictions and the Nr4a1 gene model.

FIG. 2. shows a graph of luciferase activity (y-axis) in RAW264.7 cells of 12 candidate enhancer sub-domains within Nr4a1se (x-axis). SEM shown, ** P<0.01, **** P<0.0001.

FIG. 3. shows a UCSC genome browser track showing the positions of designed sgRNAs and how they relate to the Nr4a1se sub-domains.

FIG. 4(a) shows representative flow cytometry plots showing blood monocyte frequencies in Nr4a1se_e2^{-/-}, e6^{-/-} and e9^{-/-} mice obtained from homozygous null founder mice (wild type (WT)). Gated Lin (CD3, CD19, Ly6G)⁻, CD115⁺ cells are shown stained for Ly6C (y-axis) and MHCII (x-axis). FIG. 4(b) shows a bar chart showing the reduction of Ly6C^{low} monocytes in Nr4a1se_e2 mice.

FIG. 5 shows over-represented TFBS in Nr4a1se candidate enhancer elements. Shown is a UCSC genome browser screenshot showing E02 from Nr4a1se illustrating the positions of motifs for Klf, AR and PU.1 (Spfi1) transcription factors.

FIG. 6 shows a graph of enhancer activity at Nr4a1se_2 as measured by luciferase assay. Data are plotted as the relative reporter activity of the (gene:enhancer/gene:promoter only) pair and are normalized with respect to the pEntry (no cDNA) control.

FIG. 7 shows a graph of cDNA overexpression of various Klf transcription factors in RAW264.7 cells in the presence of Nr4a1-TSS (promoter), Nr4a1-E2 (promoter+E2) and Neg (no promoter or E2) demonstrating increased E2-dependent transcription in the presence of Klf transcription factors.

FIG. 8 shows a graph of LPS-dependent activity of the Nr4a1-E2 reporter, and interaction effect with Klf2 transcription factor.

FIG. 9. Epigenomic profiling of Mo subsets and progenitors supports the model of Ly6C^{hi} to Ly6C^{low} Mo conversion. FIG. 9(a) shows a gating strategy used to sort monocytes and upstream progenitors. Cells were previously gated on live singlets (using a FSC-W/FSC-A gate). FIG. 9(b) shows UCSC genome browser screenshots showing H3K27ac and H3K4me2 tag distributions at key lineage genes. FIG. 9(c) shows distribution of H3K4me2 and H3K27ac tags±1 kb from PU.1 peak centers in DE enhancers.

FIG. 10. Ly6C^{low} Mo possess a cell-specific SE at the Nr4a1 locus. FIG. 10(a) shows a UCSC genome browser screenshot of the Nr4a1 locus depicting Ly6C^{low} Mo PU.1 and C/EBPβ binding and H3K4me2 and H3K27ac in both Ly6C^{hi} and Ly6C^{low} Mo. FIG. 10(b) and FIG. 10(c) show graphs of H3K4me2 and H3K27ac tag counts (y-axis) at Nr4a1se in MDP, cMoP, Ly6C^{hi} and Ly6C^{low} Mo as labelled on the x-axis. FIG. 10(d) shows a graphical illustration of mRNA-Seq expression levels of Nr4a1 in various tissue macrophage subsets taken from Lavin et al (2014), labelled ‘Lavin’, and from data disclosed herein, labelled ‘Thomas’. FIG. 10(e) shows H3K27ac levels at Nr4a1se for the same MP populations as in FIG. 10(d).

FIG. 11—Identification of a conserved SE sub-domain essential for Ly6C^{low} Mo development. FIG. 11(a) shows relative luciferase activity in RAW264.7 cells of candidate enhancer regions cloned into pGL4.Nr4a1 vector. Results shown are averaged over 2 independent experiments. Error bars represent SD, P-values calculated by 2-way anova * P<0.05, **P<0.01, ****P<0.0001. FIG. 1 (b) shows a UCSC genome browser screenshot of the human Nr4a1 locus. H3K27ac tracks for CD14+CD16^{dim} (classical) and CD14^{dim}CD16⁺ (non-classical) Mo are shown alongside BLAT alignments of nucleosome-free DNA sequence obtained from mouse E2, E6 and E9. Mo DNase-Seq data were taken from (Boyle et al, 2008); nucleotide sequence conservation is indicated using the GERP track. FIG. 1 (c) shows PCR Genotyping of Nr4a1se_2, Nr4a1se_6 and Nr4a1se_9 mice. FIG. 11(d) and FIG. 11(e) shows enumeration of peripheral blood Mo in Nr4a1se_2 (E2), Nr4a1se_6 (E6) and Nr4a1se_9 (E9) mice. Parametric t-tests performed relative to WT, ** p<0.01 *** p<0.001. f) FACS gating and representative flow plot for each genotype in FIG. 1 (d) and FIG. 1 (e).

FIG. 12—Nr4a1se_2 does not regulate Nr4a1 mRNA expression in response to inflammatory stimuli. FIG. 12(a) shows Nr4a1 mRNA expression levels in thioglycollate-elicited macrophages obtained from Nr4a1^{flox/flox} mice crossed to Lys2-Cre and Csf1r-Cre, WT (Cre-littermates) are shown as control. FIG. 12(b) shows a Kaplan-Mayer curve showing survival of WT, Nr4a1se_2^{-/-} and Nr4a1^{-/-} mice in response to a single dose of 2.5 mg/kg LPS I.P. Mantel-Cox (Log rank) test results: WT vs. Nr4a1^{-/-} p<0.01, Nr4a1se_2^{-/-} vs. Nr4a1^{-/-} p<0.05, Nr4a1se_2^{-/-} vs. WT p>0.05. FIG. 12(c) shows an RT-PCR time course of Nr4a1 mRNA in primary thioglycollate-elicited macrophages following LPS stimulation (100 ng/ml). FIG. 12(d) shows a Western blot showing Nr4a1 levels 1 h post LPS stimulation (100 ng/ml). FIG. 12(e) shows Nr4a1 mRNA expression in dose escalation at 1 h post LPS stimulation. FIG. 12(f) shows inflammatory cytokine mRNA expression in primary peritoneal macrophages (PM) 1 h post injection of 1 ug LPS I.P. FIG. 12(g) shows nitric oxide in PM culture supernatant 96h after LPS stimulation.

FIG. 13—Identification of motifs associated with Ly6C^{low} Mo development. FIG. 13(a) shows normalized PU.1 ChIP-Seq signal in Ly6C^{hi} and Ly6C^{low} Mo of Ly6C^{low} Mo-specific PU.1 sites ±400 bases from PU.1 peak center. FIG. 13(b) shows H3K27ac±500 bases of Ly6C^{low} Mo-specific PU.1 peaks. FIG. 13(c) shows relative expression of genes proximal to Ly6C^{low} Mo-specific PU.1 peaks. FIG. 13(d) shows overrepresented motifs in Ly6C^{low} Mo specific PU.1 peaks identified by de novo enrichment analysis. FIG. 13(e) shows RNA-Seq expression levels of TFs predicted to bind motifs in FIG. 13(d).

FIG. 14—Klf2 drives Ly6C^{hi} to Ly6C^{low} Mo conversion via Nr4a1se_2. FIG. 14(a) shows overexpression of candidate TFs in the presence of pGL4.Nr4a1_e2 and pGL4.Nr4a1 reporter vectors in RAW264.7 cells. The enhancer index is calculated as described in the methods. FIG. 14(b) shows blood Mo frequencies in Lys2-Cre Klf2^{flox/flox} and Lys2-Cre Klf4^{flox/flox} mice. FIG. 14(c) shows Nr4a1 mRNA expression levels in primary Ly6C^{hi} Mod) Klf2 mRNA correlated against Nr4a1 in Ly6C^{low} Mo. FIG. 14(e) shows the same data as FIG. 14(d) for Klf4 mRNA. FIG. 14(f) Klf2 mRNA expression levels in primary human Mo subsets as measured by microarray.

FIG. 15—Nr4a1se_2 is a monocyte-specific enhancer. FIG. 15(a) shows a gating scheme for tissue macrophage sorting. All cells were previously gated on live singlets. The side scatter high profile of lung CD11c⁺ macrophages is shown in the right panel (blue). FIG. 15(b) and FIG. 15(c) shows relative Nr4a1 mRNA expression in tissue macrophages and blood Ly6C^{hi} and Ly6C^{low} Mo. Statistics for FIG. 15(b) and FIG. 15(c) were measured by students t-test * p<0.05, ** p<0.01. FIG. 15(d) shows representative sections of B16F10 tumors in WT, Nr4a1^{-/-} and E2^{-/-} mice. FIG. 15(e) and FIG. 15(f) show quantification of cancer metastasis area. Error bars represent SD statistics were analyzed by ANOVA * p<0.05.

FIG. 16(a) shows differentially bound H3K4me2 peaks (P,1*10⁻⁵). A total of 640 enhancer regions were found to be differentially regulated between all conditions. FIG. 16(b) shows differentially bound H3K27ac peaks (P, 1*10⁻⁵). A total of 9,879 enhancer regions were found to be differentially regulated between all conditions. FIG. 16(c) shows hierarchical clustering of DE enhancer regions. Bands on the left show clusters used to define groups in FIG. 9(c). FIG. 16(d) shows the presence/absence of de novo motif instances in clusters associated with MDP, MDP/cMoP, Ly6C^{hi}/Ly6C^{low} Mo, or Ly6C^{low} Mo only, as defined in FIG. 9(c) and FIG. 16(c). Only motif instances with a significance p-value 1*10⁻⁴⁰ are shown, alongside the representative motif identified by HOMER.

FIG. 17 shows gene expression profiles Differential gene expression pairwise comparisons in mRNA-Seq data.

FIG. 18 shows a UCSC genome browser screenshot of Nr4a1 and surrounding region. The Nr4a1-associated super-enhancer (Nr4a1se) is highlighted by a vertical grey band. Super-enhancer predictions for Ly6C^{low} monocytes are shown in black directly above the gene prediction track.

FIG. 19(a) shows a UCSC genome browser screenshot showing positions of CRISPR sgRNA sites for E2, E6 and E9 KO mice. The TFs track shows superimposed PU.1 and CEBPb transcription factor binding profiles in Ly6C^{low} Mo. FIG. 19(b) shows a schematic of pGL4.10 luciferase reporter vector containing Nr4a1 300 bp of Nr4a1 promoter upstream of the TSS and the 5′ UTR sequence. FIG. 19(c) shows sgRNA design.

FIG. 20(a) shows mRNA expression levels of transcription factors belonging to family of over-represented motifs present in Ly6C^{low} monocyte enhancer regions based on RNA-Seq data. FIG. 20(b) shows Klf motifs (identified using HOMER) present in the Nr4a1 E2 enhancer sub-sequence.

FIG. 21(a) shows flow cytometric analysis of Mo subset frequencies in the bone marrow of Mac-Klf2 and Mac-Klf4 mice. FIG. 21(b) shows frequencies of GFP+ cells transduced with GFP and shRNA expressing retrovirus targeting Kf2, Klf4, or non-targeting control (NTC) sequence. FIG. 21(c) and FIG. 21(d) show Kf2 (c) and Klf4 (d) mRNA expression in blood monocyte subsets sorted from Mac-Klf2 and Mac-Klf4 mice. FIG. 21(e) FIG. 21(f) show Klf2 (e) and Klf4 (f) mRNA expression levels correlated against Nr4a1 mRNA expression in Ly6C^{hi} Mo.

FIG. 22(a) shows tissue macrophage subset frequencies in WT and Nr4a1se_2^{-/-} mice as measured by FACS. FIG. 22(b) shows blood monocyte subset frequencies in WT, Nr4a1^{-/-} and Nr4a1se_2^{-/-} mice 18 days after injection with 300,000 B16F10 melanoma cells. FIG. 22(c) shows quantification of histological sections for B16F10 tumors. FIG. 22(d) shows size distribution of individual tumors within lungs of mice relating to FIG. 15(d). Statistics for FIG. 22(c) and FIG. 22(d) were performed using students t-test, p values are shown.

### DETAILED DESCRIPTION

Enhancers regulate cell-specific patterns of gene expression and can be quantitated genome-wide using ChIP-Seq directed against the histone modification H3K4me2. Active enhancers can be further identified based on H3K27ac^{10,11}. Lineage-determining transcription factors (LDTFs) establish and maintain cell-specific enhancer repertoires. The major myeloid LDTF is PU.1¹¹. Differential PU.1 binding occurs between different macrophage populations and these cell-specific PU.1 peaks co-localize with motifs associated with transcription factors that maintain the identity of these distinct populations¹².

Disclosed herein are profiled enhancer landscapes of primary Ly6C^{hi} and Ly6C^{low} monocytes to identify factors regulating Ly6C^{low} monocyte development. The strategy integrated a molecular analysis of Nr4a1-associated enhancers with regulatory information inferred from genome-wide ChIP-Seq analysis to define high priority targets.

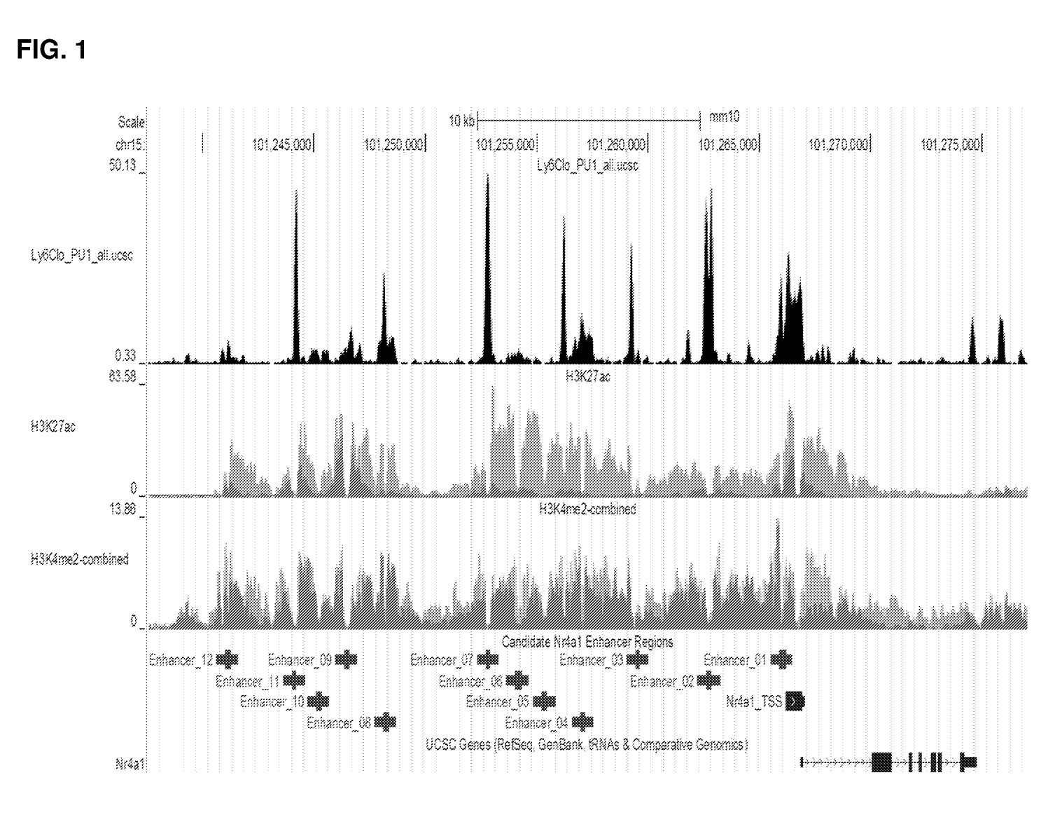

Disclosed herein are H3K4me2, H3K27ac and PU.1 ChIP-Seq data for primary mouse Ly6C^{hi} and Ly6C^{low} monocytes. Large enhancer region upstream of Nr4a1 that shows greatly increased activity in Ly6C^{low} monocytes were identified (FIG. 1). This region, termed Nr4a1se, displays the characteristics of a super-enhancer. Super-enhancers typically span >10 kb and are found nearby, and control the expression of, genes important for cell-type specification^{13,14}. Thus it was found that Nr4a1se regulates Nr4a1 in Ly6C^{low} monocytes.

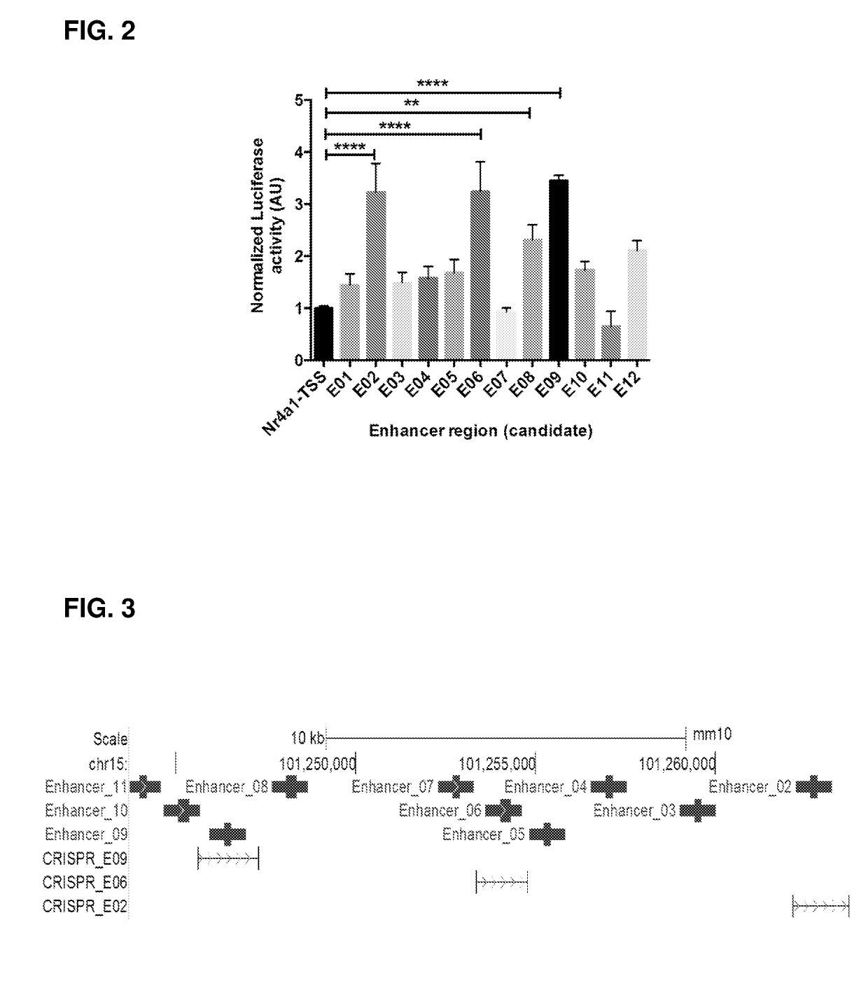

To characterize the regions of Nr4a1se that are functionally relevant for transcriptional regulation 12 regions defined on the basis of nucleosome depletion and PU.1 occupancy, two measures of active transcription factor binding¹¹, were cloned. Luciferase reporters consisting of the Nr4a1 promoter and each of these 12 candidate loci (FIGS. 1 and 2) were tested for enhancer activity using the macrophage like RAW264.7 cell line. Regions E02, E06 and E09 were found to display basal enhancer activity (FIG. 2). A large fold-change induction of these enhancers in RAW264.7 cells was not expected as the requisite transcription factors are expected to be specific to the Ly6C^{low} monocyte subset.

Enhancer landscapes of both human CD14⁺CD16⁻ and CD14^{dim}CD16⁺ monocytes have been reported¹⁵. Although not wishing to be bound to or limited by any theory, if the human CD14^{dim}CD16⁺ monocyte population is truly orthologous to the mouse Ly6C^{low} monocyte subset then the transcriptional regulation of lineage-determining transcription factors would be subject to conservation by natural selection. Using a comparative genomics approach it was found that orthologous human DNA sequence to mouse regions E02, E06 and E09 contain elevated H3K27ac in the CD14^{dim}CD16⁺ Ly6C^{low} monocyte equivalent.

As disclosed herein, it was discovered that E02, E06 and E09 are functional, evolutionarily conserved, sequences relevant for Nr4a1 gene expression in Ly6C^{low} monocytes. Using the CRISPR-Cas9 system^{16,17} mice were made containing deletions of each of these regions to test their role in Ly6C^{low} monocyte development. CRISPR-Cas9 is a nuclease complex that uses an RNA oligonucleotide (sgRNA) to direct the Cas9 nuclease to the site of genomic DNA cleavage with high precision. Administration of two sgRNA molecules alongside Cas9 results in pairs of DNA double-stranded breaks that are fused by the non-homologous end-joining DNA repair pathway¹⁸. As outlined in FIG. 3 this approach has been used to make Nr4a1se_2, 6 and 9^{-/-} mice respectively.

As disclosed herein, it was discovered by sequencing that the desired deletions are present, and multiple instances of germline transmission have been observed for each strain.

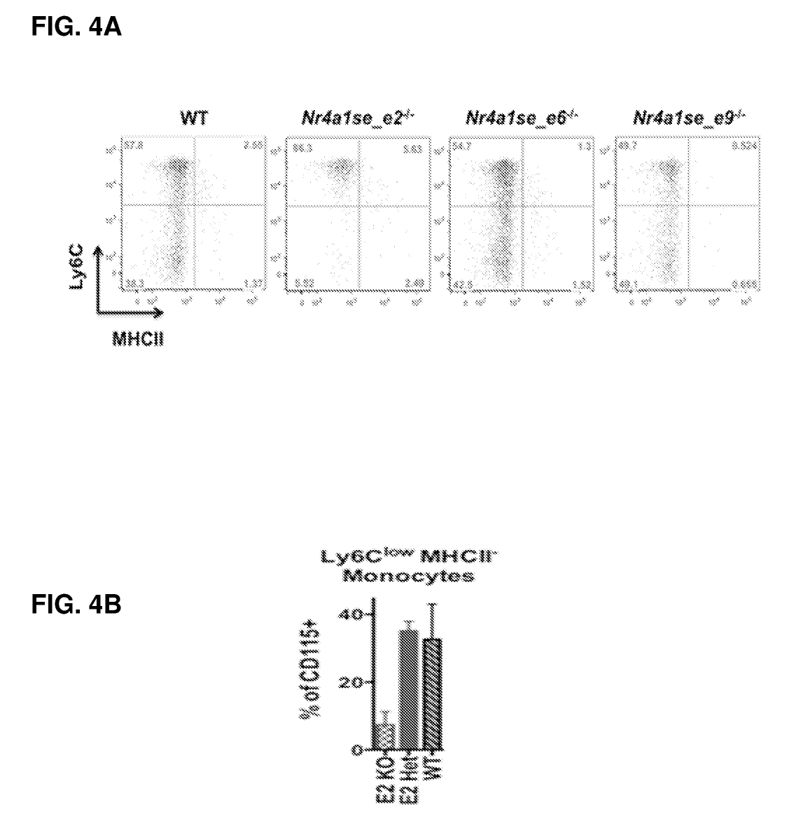

Global Nr4a1^{-/-} mice lack Ly6C^{low} monocytes, and therefore blood monocyte population frequencies in Nr4a1se_2, e6 and e9^{-/-} founder mice were assessed. Excitingly it was found that a single region, Nr4a1se_2, is required for the Ly6C^{low} monocyte subset in vivo (FIG. 4a, b). These findings are consistent with the hypothesis that a single, or small number, of transcription factors regulate Ly6C^{low} monocyte commitment by converging at a single genomic locus.

Without being bound to or limited by any particular theory, the transcription factors driving Nr4a1 expression in Ly6C^{low} monocytes may impart a global transcriptional signature within Ly6C^{low} monocyte-specific enhancers. De novo motif analysis of Ly6C^{low} monocyte-associated enhancers identified several motifs including those for PU.1 and Nur77.

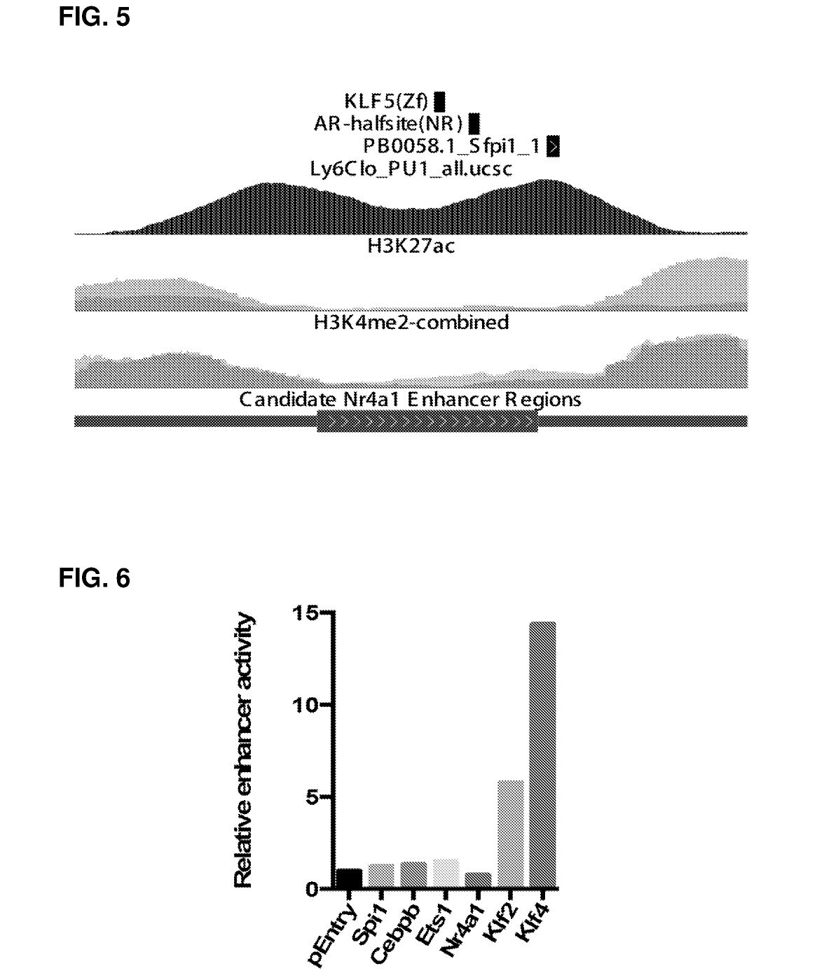

As also disclosed herein, motifs for CEBP, RUNX, KLF, MEF2, AP-1, AR/E2F and SMAD4 transcription factors were identified. These de novo motifs were mapped to Nr4a1se_2 sequence to identify candidate motifs driving transcriptional activity at this region. Instances of KLF, AR and PU.1 motifs were identified in this region (FIG. 5). In addition, a lower confidence Nf-κB motif associated with Ly6C^{low} monocytes that was present in the E2 region has been identified.

As disclosed herein, it was determined that transcription factors that bind to motifs identified in FIG. 5 regulate Nr4a1 transcription in Ly6C^{low} monocytes. RNA-Seq gene expression data was interrogated to find transcription factors that are both expressed in Ly6C^{low} monocytes and up-regulated in Ly6C^{low} relative to Ly6C^{hi} monocytes. Both monocyte subsets express multiple transcription factors that can bind to these motifs Specifically these transcription factors are Spi1 (PU.1), Ets1 (PU.1), Spib (PU.1), Nr4a1 (AR-halfsite), Klf2 (KLF), Klf4 (KLF), Klf6 (KLF), Klf10 (KLF), Klf13 (KLF).

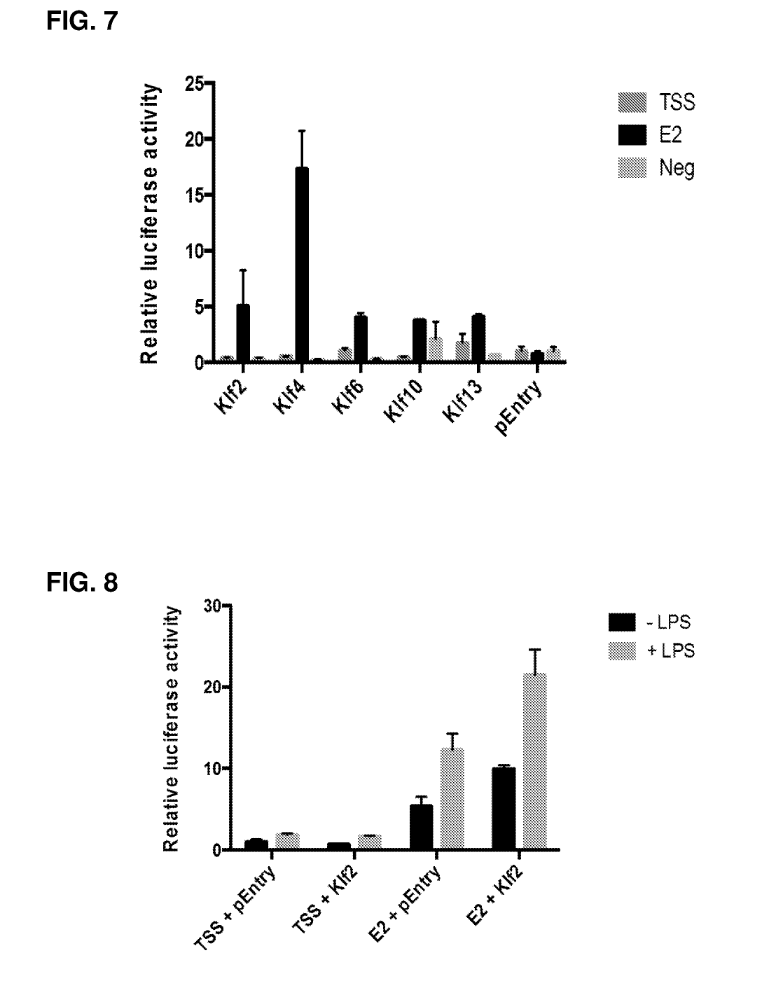

An in vitro approach was taken to identify transcription factors that act via Nr4a1se_2 enhancer element. cDNA clones (TrueORF, Origene) for each of the transcription factors defined above were transfected into macrophage-like RAW264.7 cells alongside the Nr4a1se_2 reporter vector. RAW264.7 cells are a well established workhorse for macrophage transcriptional regulation assays. As background controls the Nr4a1-TSS region without E2 enhancer sequence was used, and a vector containing neither the Nr4a1-TSS promoter or enhancer region. FIGS. 6 and 7 show the results from the study and cDNA overexpression of each of the transcription factors in the presence of the E2 enhancer sequence. These data clearly show that Klf family transcription factors, and klf2 and Klf4 in particular drive the expression of Nr4a1-luciferase activity.

As disclosed herein, also identified was an Nf-κB site within the E2 region and tested if this region is responsive to Nf-κB signalling. For this study the E2 reporter (and TSS-only, and negative control plasmid) was treated with the Tlr4 ligand LPS, a potent regulator of Nf-κB activity. The results from this study (FIG. 8) show that the E2 region is also LPS responsive, and that there is a synergistic effect between Klf2 and Nf-κB at the E2 locus.

As disclosed herein, mechanisms that regulate the expression of Nr4a1 in Ly6C^{low} monocytes (via the Nr4a1se_2 region) represent a druggable target. Agonising this pathway increases Ly6C^{low} monocyte numbers, antagonising the pathway blocks the production of these cells. Without being be bound to or limited by to any particular theory, Klf transcription factors and Nf-κB may play crucial roles in this process.

Thus in one embodiment, there is provided a novel approach to affect (promote/inhibit) non-classical (Ly6C^{low} in mouse and CD14^{dim}CD16⁺ in human) monocyte development. By modulating the activity of transcription factors that bind to the DNA sequence in the region Nr4a1se_2 in Ly6C^{low} monocytes, or upstream progenitor cell types (classical monocyte, cMoP, MDP), the transcription activity of Nr4a1 can be modulated. In turn this affects the activity of this cell type, which is of therapeutic importance, including in inflammation and wound healing. In certain embodiments of the provided methods, CD14^{dim}CD16⁺ (CD115⁺CD11b⁺GR1⁻ (Ly6C−)) monocytes or macrophages are modulated by modulating Nf-κB. In particular embodiments of the invention methods, Nf-κB is modulated in combination with modulating transcription factors.

In some embodiments, disclosed herein is a method of modulating the activity of CD14^{dim}CD16⁺ monocyte or macrophage cell production development, survival, proliferation differentiation or activity. Methods of modulating monocytes or macrophage can be in vitro methods or in vivo methods. In vitro methods often comprise contacting one or more cells, or portions thereof, with an agonist or antagonist. In vivo methods often comprise providing or administering an agonist or antagonist to a subject (e.g., a mammal, a human).

In certain embodiments, an agonist or antagonist is an antibody, or binding fragment thereof. In some embodiments an antibody, or binding fragments thereof, binds specifically to KLf2, Klf4 or Nf-κB. Accordingly, an antagonist antibody, or binding fragment thereof can bind specifically to Klf2, Klf4 or Nf-κB, which binding can inhibit or block the transcriptional regulatory activity of Klf2, Klf4 or Nf-κB. For example, specific binding of an antagonist antibody to Klf2 can inhibit or block binding of Klf2 to its DNA binding site or inhibit or block the transcriptional regulatory activity of Klf2. Alternatively, an agonist antibody, or binding fragment thereof can bind specifically to Klf2, Klf4 or Nf-κB, which binding can promote, increase, stimulate or induce the transcriptional activity of Klf2, Klf4 or Nf-κB. For example, specific binding of an agonist antibody to Klf2 can promote, increase, stimulate or induce binding of Klf2 to its DNA binding site or promote, increase, stimulate or induce the transcriptional regulatory activity of Klf2. Methods of identifying, selecting and/or assaying the agonist or antagonist activity of an antibody that specifically binds to Klf2, Klf4 or Nf-κB are disclosed herein.

In some embodiments, an agonist or antagonist antibody is administered to a subject. In certain embodiments, a subject having a disease or disorder (e.g., lymphoproliferative disorder (e.g., a cancer)) is treated for said disorder by administering to the subject an agonist or antagonist antibody. In certain embodiments, a subject having an autoimmune disorder or disease is treated for said disorder by administering to the subject an agonist or antagonist antibody.

The terms “antagonist” and the like refer to an agent which reduces the level of activity of a transcription factor or the level of expression of a transcription factor, e.g., Klf2 or Klf4. An antagonist can be a binding agent, a small molecule inhibitor, an allosteric inhibitor, an antibody, an inhibitory nucleic acid, an RNAi molecule, or a ligand mimetic.

The terms “subject,” “patient,” “individual,” etc. are not intended to be limiting and can be generally interchanged. That is, an individual described as a “patient” does not necessarily have a given disease, but may be merely seeking medical advice.

As used herein, the terms “treat” and “prevent” may refer to any delay in onset, reduction in the frequency or severity of symptoms, amelioration of symptoms, improvement in patient comfort or function (e.g. nt function), decrease in severity of the disease state, etc. The effect of treatment can be compared to an individual or pool of individuals not receiving a given treatment, or to the same patient prior to, or after cessation of, treatment. The term “prevent” generally refers to a decrease in the occurrence of a given disease (e.g. an autoimmune, inflammatory autoimmune, cancer, infectious, immune, or other disease) or disease symptoms in a patient. As indicated above, the prevention may be complete (no detectable symptoms) or partial, such that fewer symptoms are observed than would likely occur absent treatment.

“Nucleic acid” or “oligonucleotide” or “polynucleotide” or grammatical equivalents used herein means at least two nucleotides covalently linked together. The term “nucleic acid” includes single-, double-, or multiple-stranded DNA, RNA and analogs (derivatives) thereof. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, or even longer. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

A particular nucleic acid sequence also encompasses “splice variants.” Similarly, a particular protein encoded by a nucleic acid encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher, et al., J. Biol. Chem. 273(52):35095-35101 (1998).

An “inhibitory nucleic acid” is a nucleic acid (e.g. DNA, RNA, polymer of nucleotide analogs) that is capable of binding to a target nucleic acid (e.g. an mRNA translatable into a transcription factor) and reducing transcription of the target nucleic acid (e.g. mRNA from DNA) or reducing the translation of the target nucleic acid (e.g., mRNA) or altering transcript splicing (e.g. single stranded morpholino oligo). In certain embodiments, the “inhibitory nucleic acid” is a nucleic acid that is capable of binding (e.g. hybridizing) to a target nucleic acid (e.g. an mRNA translatable into a transcription factor) and reducing translation of the target nucleic acid. The target nucleic acid is or includes one or more target nucleic acid sequences to which the inhibitory nucleic acid binds (e.g. hybridizes). Thus, an inhibitory nucleic acid typically is or includes a sequence (also referred to herein as an “antisense nucleic acid sequence”) that is capable of hybridizing to at least a portion of a target nucleic acid at a target nucleic acid sequence. An example of an inhibitory nucleic acid is an antisense nucleic acid. Another example of an inhibitory nucleic acid is siRNA or RNAi (including their derivatives or pre-cursors, such as nucleotide analogs). Further examples include shRNA, miRNA, shmiRNA, or certain of their derivatives or pre-cursors. In some embodiments, the inhibitory nucleic acid is single stranded. In some embodiments, the inhibitory nucleic acid is double stranded.

An “antisense nucleic acid” is a nucleic acid (e.g. DNA, RNA or analogs thereof) that is at least partially complementary to at least a portion of a specific target nucleic acid (e.g. a target nucleic acid sequence), such as an mRNA molecule (e.g. a target mRNA molecule) (see, e.g., Weintraub, Scientific American, 262:40 (1990)), for example antisense, siRNA, shRNA, shmiRNA, miRNA (microRNA). Thus, antisense nucleic acids are capable of hybridizing to (e.g. selectively hybridizing to) a target nucleic acid (e.g. target mRNA). In some embodiments, the antisense nucleic acid hybridizes to the target nucleic acid sequence (e.g. mRNA) under stringent hybridization conditions. In some embodiments, the antisense nucleic acid hybridizes to the target nucleic acid (e.g. mRNA) under moderately stringent hybridization conditions. Antisense nucleic acids may comprise naturally occurring nucleotides or modified nucleotides such as, e.g., phosphorothioate, methylphosphonate, and -anomeric sugar-phosphate, backbone-modified nucleotides.

In some embodiments, an antagonist is an inhibitory nucleic acid. In certain embodiments, an inhibitory nucleic acid is an inhibitory RNA (iRNA), non-limiting examples of which include siRNA, RNAi, shRNA, miRNA, shmiRNA, morpholino oligos, the like, including derivatives and pre-cursors thereof, such as those comprising nucleotide analogs, and combinations thereof. An iRNA can mediate the targeted cleavage of an RNA transcript via an RNA-induced silencing complex (RISC) pathway. An iRNA can direct the sequence-specific degradation of mRNA through a process known as RNA interference (RNAi). In some embodiments, an iRNA modulates (e.g., inhibits, suppresses, blocks) the expression of a Klf2, Klf4 or Nf-κB polypeptide in a cell (e.g., a cell within a subject).

In some embodiments an iRNA includes a single stranded RNA that interacts with a target RNA sequence (e.g., Klf2, Klf4 or Nf-κB target mRNA sequence), which interaction directs cleavage or degradation of the target RNA. In another embodiment, an iRNA is a single-stranded siRNA that is introduced into a cell or organism to inhibit a target mRNA. Single-stranded siRNAs are generally 15-100 or 15-30 nucleotides in length and are sometimes chemically modified. The design and testing of single-stranded siRNAs are described in U.S. Pat. No. 8,101,348 and in Lima et al., (2012) Cell 150: 883-894.

In some embodiments, an iRNA is a single-stranded antisense RNA molecule that inhibits a target via an antisense inhibition mechanism. A single-stranded antisense RNA molecule is often complementary to a sequence within a target mRNA. Antisense RNA can inhibit translation in a stoichiometric manner by base pairing to a target mRNA and physically obstructing the translation machinery, see Dias, N. et al., (2002) Mol Cancer Ther 1:347-355. Alternatively, a single-stranded antisense RNA molecule can inhibit a target mRNA by hydridizing to a target mRNA and cleaving the target through an RNaseH cleavage event. A single-stranded antisense RNA molecule may be of any suitable length. In some embodiments, an antisense RNA is about 15 to about 30 nucleotides, or 30 nucleotides or more, in length and has a sequence that is completely or partially complementary to a target mRNA sequence.

In some embodiments, an iRNA is a double-stranded RNA and is referred to herein as a dsRNAi agent. A dsRNAi agent is often a complex of ribonucleic acid molecules, having a duplex structure comprising two anti-parallel and substantially complementary nucleic acid strands, referred to as having “sense” and “antisense” orientations with respect to a target RNA. In some embodiments of the invention, a dsRNAi agent triggers the degradation of a target RNA, e.g., an mRNA, through a post-transcriptional gene-silencing mechanism referred to herein as RNA interference or RNAi.

An iRNA generally can comprise one or two strands. In some embodiments, an iRNA comprises one strand of iRNA. In certain embodiments, an iRNA comprises two substantially complementary strands. In some embodiments, two strands of an iRNA are each separate RNA molecules. In some embodiments, two strands of an iRNA are covalently connected by a linker.

In certain embodiments, an iRNA comprises a morpholino oligo. In some embodiments, an antisense nucleic acid is a morpholino oligo. In some embodiments, a morpholino oligo is a single stranded antisense nucleic acid, as is know in the art. In some embodiments, a morpholino oligo decreases protein expression of a target, reduces translation of the target mRNA, reduces translation initiation of the target mRNA, or modifies transcript splicing. In some embodiments, the morpholino oligo is conjugated to a cell permeable moiety (e.g. peptide). Antisense nucleic acids may be single or double stranded nucleic acids.

In the cell, the antisense nucleic acids may hybridize to the target mRNA, forming a double-stranded molecule. The antisense nucleic acids interfere with the translation of the mRNA, since the cell will not translate a mRNA that is double-stranded. The use of antisense methods to inhibit the in vitro translation of genes is well known in the art (Marcus-Sakura, Anal. Biochem., 172:289, (1988)). Antisense molecules which bind directly to the DNA may be used.

Inhibitory nucleic acids can be delivered to the subject using any appropriate means known in the art, including by injection, inhalation, or oral ingestion. Another suitable delivery system is a colloidal dispersion system such as, for example, macromolecule complexes, nanocapsules, microspheres, beads, and lipid-based systems including oil-in-water emulsions, micelles, mixed micelles, and liposomes. An example of a colloidal system is a liposome. Liposomes are artificial membrane vesicles which are useful as delivery vehicles in vitro and in vivo. Nucleic acids, including RNA and DNA within liposomes and be delivered to cells in a biologically active form (Fraley, et al., Trends Biochem. Sci., 6:77, 1981). Liposomes can be targeted to specific cell types or tissues using any means known in the art. Inhibitory nucleic acids (e.g. antisense nucleic acids, morpholino oligos) may be delivered to a cell using cell permeable delivery systems (e.g. cell permeable peptides). In some embodiments, inhibitory nucleic acids are delivered to specific cells or tissues using viral vectors or viruses.

An “siRNA” refers to a nucleic acid that forms a double stranded RNA, which double stranded RNA has the ability to reduce or inhibit expression of a gene or target gene when the siRNA is present (e.g. expressed) in the same cell as the gene or target gene. The siRNA is typically about 5 to about 100 nucleotides in length, more typically about 10 to about 50 nucleotides in length, more typically about 15 to about 30 nucleotides in length, most typically about 20-30 base nucleotides, or about 20-25 or about 24-29 nucleotides in length, e.g., 20, 21, 22, 23, 24, 25, 26, 27, 28, 29, or 30 nucleotides in length. siRNA molecules and methods of generating them are described in, e.g., Bass, 2001, Nature, 411, 428-429; Elbashir et al., 2001, Nature, 411, 494-498; WO 00/44895; WO 01/36646; WO 99/32619; WO 00/01846; WO 01/29058; WO 99/07409; and WO 00/44914. A DNA molecule that transcribes dsRNA or siRNA (for instance, as a hairpin duplex) also provides RNAi. DNA molecules for transcribing dsRNA are disclosed in U.S. Pat. No. 6,573,099, and in U.S. Patent Application Publication Nos. 2002/0160393 and 2003/0027783, and Tuschl and Borkhardt, Molecular Interventions, 2:158 (2002).

The siRNA can be administered directly or siRNA expression vectors can be used to induce RNAi that have different design criteria. A vector can have inserted two inverted repeats separated by a short spacer sequence and ending with a string of T's which serve to terminate transcription.

Construction of suitable vectors containing the desired therapeutic gene coding and control sequences employs standard ligation and restriction techniques, which are well understood in the art (see Maniatis et al., in Molecular Cloning: A Laboratory Manual, Cold Spring Harbor Laboratory, New York (1982)). Isolated plasmids, DNA sequences, or synthesized oligonucleotides are cleaved, tailored, and re-ligated in the form desired.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles disclosed herein.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. In some embodiments, antibodies or fragments of antibodies may be derived from different organisms, including humans, mice, rats, hamsters, camels, etc. Antibodies disclosed herein may include antibodies that have been modified or mutated at one or more amino acid positions to improve or modulate a desired function of the antibody (e.g. glycosylation, expression, antigen recognition, effector functions, antigen binding, specificity, etc.).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}−C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of suitable antibodies as disclosed herein and for use according to the methods disclosed herein, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides as disclosed herein. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Typical antibodies of, and for use according to the invention include humanized and/or chimeric monoclonal antibodies.

In some embodiments, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein. Such effector moieties include, but are not limited to, an anti-tumor drug, a toxin, a radioactive agent, a cytokine, a second antibody or an enzyme.

The immunoconjugate can be used for targeting the effector moiety to a cell, e.g. CD14dimCD16+ monocytes, assay of which can be readily apparent when viewing the bands of gels with approximately similarly loaded with test and controls samples. Examples of cytotoxic agents include, but are not limited to ricin, doxorubicin, daunorubicin, taxol, ethidium bromide, mitomycin, etoposide, tenoposide, vincristine, vinblastine, colchicine, dihydroxy anthracin dione, actinomycin D, diphteria toxin, Pseudomonas exotoxin (PE) A, PE40, abrin, and glucocorticoid and other chemotherapeutic agents, as well as radioisotopes. Suitable detectable markers include, but are not limited to, a radioisotope, a fluorescent compound, a bioluminescent compound, chemiluminescent compound, a metal chelator or an enzyme.

Additionally, the recombinant proteins disclosed herein including the antigen-binding region of any of the antibodies disclosed herein can be used to treat inflammation. In such a situation, the antigen-binding region of the recombinant protein is joined to at least a drug having therapeutic activity. The second drug can include, but is not limited to, a nonsteroidal anti-inflammatory drug. Suitable nonsteroidal anti-inflammatory drugs include aspirin, celecoxib (Celebrex), diclofenac (Voltaren), diflunisal (Dolobid), etodolac (Lodine), ibuprofen (Motrin), indomethacin (Indocin), ketoprofen (Orudis), ketorolac (Toradol), nabumetone (Relafen), naproxen (Aleve, Naprosyn), oxaprozin (Daypro), piroxicam (Feldene), salsalate (Amigesic), sulindac (Clinoril), tolmetin (Tolectin).

Techniques for conjugating therapeutic agents to antibodies are well known (see, e.g., Arnon et al., “Monoclonal Antibodies For Immunotargeting Of Drugs In Cancer Therapy”, in MONOCLONAL ANTIBODIES AND CANCER THERAPY, Reisfeld et al. (eds.), pp. 243-56 (Alan R. Liss, Inc. 1985); Hellstrom et al., ANTIBODIES FOR DRUG DELIVERY IN CONTROLLED DRUG DELIVERY (2nd Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)).

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

An “RNAi molecule” is an siRNA, shRNA, miRNA, shmiRNA, or other nucleic acid, as well known in the art, that is capable of inducing RNAi and hybridizing to an RNA that is translatable to a transcription factor. The RNAi molecule is typically capable of decreasing the amount of transcription factor that is translated in a cell.

A “ligand mimetic” is a binding agent that is designed to mimic, in structure or in binding mode, a known ligand or is capable of inhibiting the binding of a natural or physiological ligand to a transcription factor. In some embodiments, a ligand mimetic is a synthetic chemical compound, peptide, protein, fusion protein, peptidomimetic, or modified natural ligand. For example, a ligand mimetic may bind the same amino acids or a subset of the same amino acids on a transcription factor that a natural ligand of the transcription factor binds during the physiological functioning of the transcription factor. Ligand mimetics include biopolymers (e.g. proteins, nucleic acids, or sugars), lipids, chemical molecules with molecular weights less than five hundred (500) Daltons, one thousand (1000) Daltons, five thousand (5000) Daltons, less than ten thousand (10,000) Daltons, less than twenty five thousand (25,000) Daltons, less than fifty thousand (50,000) Daltons, less than seventy five thousand (75,000), less than one hundred thousand (100,000), or less than two hundred fifty thousand (250,000) Daltons. In some embodiments, the synthetic chemical compound is greater than two hundred fifty thousand (250,000) Daltons. In certain embodiments, the binding agent is less than five hundred (500) Daltons. In some embodiments, a ligand mimetic is a protein.

In some embodiments, a ligand mimetic is a small chemical molecule. The term “small chemical molecule” and the like, as used herein, refers to a molecule that has a molecular weight of less than two thousand (2000) Daltons. In some embodiments, a small chemical molecule is a molecule that has a molecular weight of less than one thousand (1000) Daltons. In other embodiments, a small chemical molecule is a molecule that has a molecular weight of less than five hundred (500) Daltons. In other embodiments, a small chemical molecule is a molecule that has a molecular weight of less than five hundred (500) Daltons. In other embodiments, a small chemical molecule is a molecule that has a molecular weight of less than one hundred (100) Daltons.

In some embodiments, a transcription factor inhibitor is a small molecule. In other embodiments the inhibitor is an allosteric inhibitor of a transcription factor.

In some embodiments, there is provided herein a method of modulating CD14^{dim}CD16⁺ (CD115⁺CD11b⁺GR1⁻ (Ly6C⁻)) monocytes or macrophages, the method comprising modulating expression or activity of Nr4a1 (Nur77). In some embodiments, there is provided herein a method of modulating an amount of CD14^{dim}CD16⁺ (CD115⁺CD11b⁺GR1⁻ (Ly6C⁻)) monocytes or macrophages in a subject. In some embodiments, provided herein is a method of increasing or decreasing an amount of CD14^{dim}CD16⁺ (CD115⁺CD11b⁺GR1⁻ (Ly6C⁻)) monocytes or macrophages in a subject, the method comprising administering an agonist or antagonist described herein. An amount of monocytes or macrophages can be increased or decreased from about 2-fold to 10,000 fold or more relative to an amount of monocytes or macrophages existing in a subject prior to administering an agent (e.g., an agonist or antagonist) described herein. In certain embodiments, disclosed herein is a method of stimulating, promoting, increasing or inducing CD14^{dim} CD16⁺ monocyte or macrophage cell production, development, survival, proliferation, differentiation or activity, comprising modulating expression or activity of a transcription factor that regulates expression of Nr4a1 (Nur77).

In certain embodiments, provided herein is a method of identifying an agent that modulates CD14^{dim}CD16⁺ (CD115⁺CD11b⁺GR1⁻ (Ly6C⁻)) monocytes or macrophages. In certain embodiments, disclosed herein is a method of identifying an agent that modulates the amount of CD14^{dim}CD16⁺ (CD115⁺CD11b⁺GR1⁻ (Ly6C⁻)) monocytes or macrophages in a subject. In certain embodiments, disclosed herein is a method of identifying an agent that increases or decreases an amount of CD14^{dim}CD16⁺ (CD115⁺CD11b⁺GR1⁻ (Ly6C⁻)) monocytes or macrophages in a subject. An agent can be an antibody, a small molecule, a peptide, a polypeptide or protein, an inhibitory nucleic acid, an allosteric inhibitor or a ligand mimetic. In certain embodiments, an agent is an agonist or antagonist of Nr4a1 activity or expression. In certain embodiments, an agent is an agonist or antagonist of Klf2 or Klf4 activity or expression.

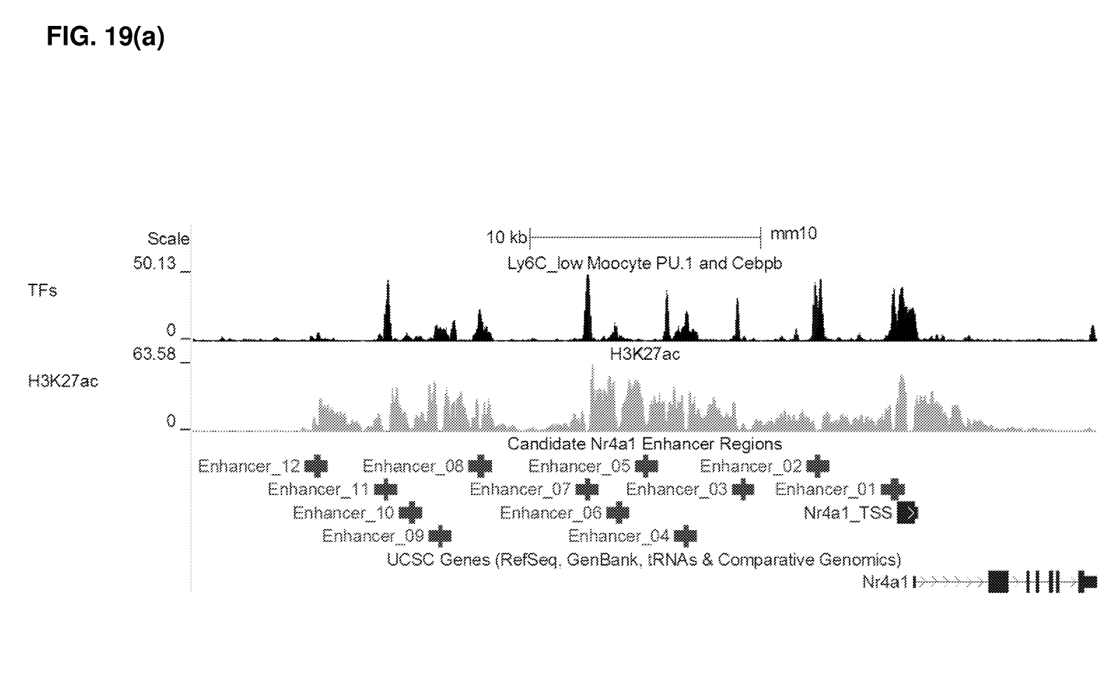

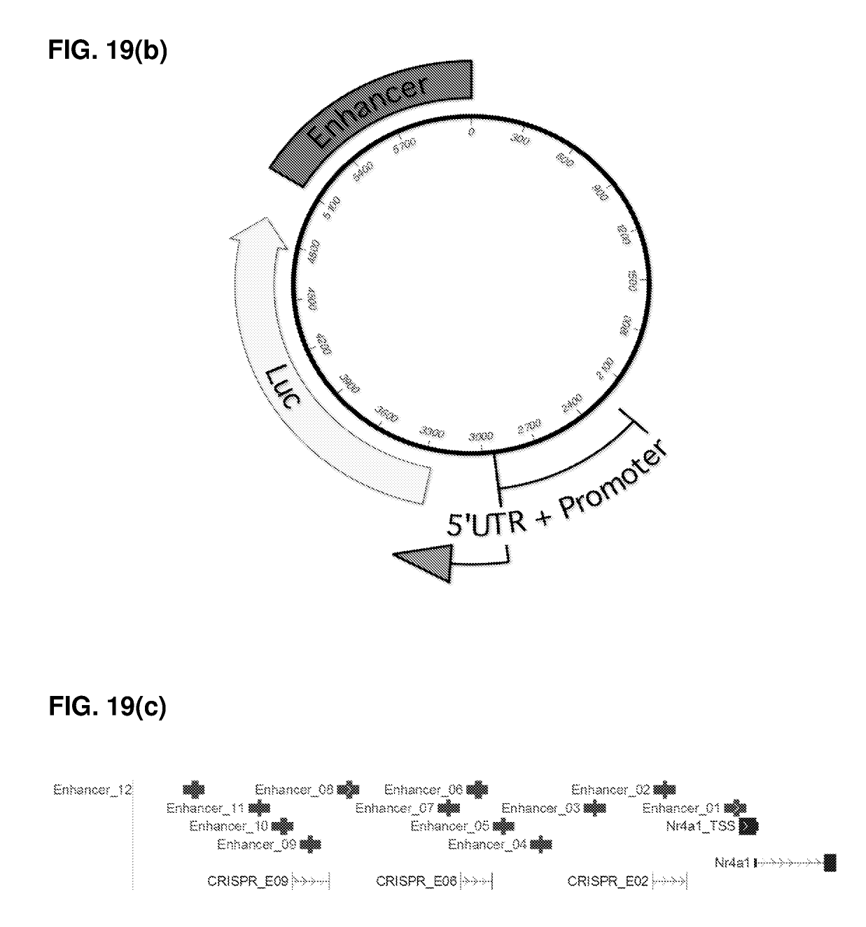

In some embodiments, a method of identifying an agent that modulates, increases or decreases CD14^{dim}CD16⁺ (CD115⁺CD11b⁺GR1⁻ (Ly6C⁻)) monocyte or macrophage amounts or activity comprises identifying an agent that modulates, increases, or decreases Nr4a1expression. In some embodiments, a method of identifying an agent that modulates, increases or decreases CD14^{dim}CD16⁺ (CD115⁺CD11b⁺GR1⁻ (Ly6C⁻)) monocyte or macrophage amounts or activity comprises identifying an agent that modulates, increases or decreases activity or expression of a transcription factor that regulates expression of Nr4a1 (Nur77). In some embodiments, a method of identifying an agent that modulates, increases or decreases CD14^{dim}CD16⁺ (CD115⁺CD11b⁺GR1⁻ (Ly6C⁻)) monocyte or macrophage amounts or activity comprises identifying an agent that modulates, increases or decreases activity or expression of Klf2, Klf4 and/or Nf-κB. In certain embodiments, agents that modulate, increase or decrease expression Nr4a1 can be identified by monitoring the activity of a transcriptional regulatory region of Nr4a1, which can be operably linked to a reporter sequence. In one non-limiting example, an experimental agent can be contacted with Klf2, Klf4 and/or Nf-κB in the presence of an Nr4a1 transcriptional regulatory region (e.g., any one or more of Enhancer_01 to Enhancer_12, e.g., see FIG. 19) operably linked to a suitable reporter. Expression or activity of the reporter can be monitored to determine if the experimental agent decreases or increases reporter activity. In some embodiments, a method of identifying an agent that modulates, increases or decreases CD14^{dim}CD16⁺ (CD115⁺CD11b⁺GR1⁻ (Ly6C⁻)) monocyte or macrophage amounts or activity comprises identifying an agent that modulates, increases, or decreases Nr4a1expression in the presence of Klf2, Klf4 and/or Nf-κB.

In some embodiments, a method of identifying an agent that modulates, increases or decreases CD14^{dim}CD16⁺ (CD115⁺CD11b⁺GR1⁻ (Ly6C⁻)) monocyte or macrophage amounts or activity comprises contacting an experimental agent with an Nr4a1 promoter region. An Nr4a1 promoter region can be a nucleic acid region 20 base pairs (bp) to 1500 bp in length located within the 5′ untranslated region (UTR) the Nr4a1 gene (HGNC: 7980, Entrez Gene: 3164, Ensembl: ENSG00000123358). In some embodiments, an Nr4a1 promoter region is 20 bp to 1000 bp, 20 bp to 750 bp, 20 bp to 500 bp, 20 bp to 250 bp or 20 bp to 100 bp in length. In certain embodiments, an Nr4a1 promoter region comprises about 20-1500 base pairs 5′ of the Nr4a1 transcriptional start site, or a subsequence thereof. In certain embodiments, an Nr4a1 promoter region comprises an enhancer region (e.g., enhancer_01 (Nr4a1se_1), 02 (Nr4a1se_2), 03 (Nr4a1se_3), 04 (Nr4a1se_4), 05 (Nr4a1se_5), 06 (Nr4a1se_6), 07 (Nr4a1se_7), 08 (Nr4a1se_8), 09 (Nr4a1se_9), 10 (Nr4a1se_10), 11 (Nr4a1se_11), 12 (Nr4a1se_12), or a combination thereof, e.g., see FIG. 19). In certain embodiments an Nr4a1 promoter region comprises an enhancer region isolated by a set of primer sequences shown in Table 1, or an Nr4a1 promoter region isolated as described herein (e.g., see section entitled “Enhancer Cloning”). In certain embodiments, an Nr4a1 promoter region comprises or consists of enhancer site E2 (Nr4a1se_2), E6 (Nr4a1se_6) or E9 (Nr4a1se_9) as disclosed herein.

Any suitable methods of detecting enhancer/promoter activity can be used to detect enhancer/promoter activity or transcription factor activity. In certain embodiments, an Nr4a1 promoter region, enhancer region, subsequence or combination thereof is operably linked to a suitable reporter nucleic acid. Reporter nucleic acids can be detected directly upon transcription, or indirectly upon translation of a suitable reporter sequence. A multitude of suitable reporter nucleic acids and suitable reporter polypeptides are known, any of which can be used for a method herein. In some embodiments, a reporter nucleic acid is a nucleic acid encoding Nr4a1. In some embodiments, a reporter nucleic acid is a nucleic acid encoding a luciferase. Expression of a reporter nucleic acid can be determined indirectly by measuring the amount or activity of a polypeptide encoded by a reporter nucleic acid (e.g., a reporter polypeptide).

In some embodiments, a method of identifying an agent that modulates, increases or decreases CD14^{dim}CD16⁺ (CD115⁺CD11b⁺GR1⁻ (Ly6C⁻)) monocyte or macrophage amounts or activity comprises contacting an experimental agent with an Nr4a1 promoter region operably linked to a reporter nucleic acid, in the presence of a transcription factor, non-limiting examples of which include Klf2, Klf4, and Nf-κB. Any suitable control can be used for a method herein. In certain embodiments a control assay is an experimental method performed in the absence of an experimental agent. Non-limiting examples of experimental agents include antibodies, inhibitory nucleic acids, small molecules, peptide, polypeptides, proteins, allosteric inhibitors, ligand mimetics, the like or combinations thereof. In certain embodiments an experimental agent binds specifically to Klf2, Klf4 or an Nf-κB polypeptide. In certain embodiments an experimental agent binds specifically to a nucleic acid encoding an Klf2, Klf4 or an Nf-κB polypeptide.

In certain embodiments, an agent that modulates, increases or decreases CD14^{dim}CD6⁺ (CD115⁺CD11b⁺GR1⁻ (Ly6C⁻)) monocyte or macrophage amounts or activity can be identified by comparing the expression, amount or activity of a reporter nucleic acid or reporter polypeptide produced in the presence of an experimental agent to an amount or activity of a reporter nucleic acid or reporter polypeptide produced in the absence of an experimental agent (e.g., a control). In certain embodiments, an experimental agent that increases the amount or activity of a reporter nucleic acid or reporter polypeptide relative to a control amount produced in the absence of an experimental agent is an agent that modulates or increases CD14^{dim}CD16+(CD115+CD11b+GR1⁻ (Ly6C⁻)) monocyte or macrophage amounts or activity. In certain embodiments, an experimental agent that decreases the amount or activity of a reporter nucleic acid or reporter polypeptide relative to a control amount produced in the absence of an experimental agent is an agent that modulates or decreases CD14^{dim}CD16⁺ (CD115⁺CD11b⁺GR1⁻ (Ly6C⁻)) monocyte or macrophage amounts or activity.

In some embodiments, a vertebrate, (e.g., a mammal, e.g., a rodent; e.g., a rat, e.g., a mouse, or the like) deficient in an Nr4a1se_2 sequence is generated. A vertebrate deficient in an Nr4a1se_2 sequence can be homozygous or heterozygous for a deleted, modified or disrupted Nr4a1se_2A sequence. A vertebrate deficient in an Nr4a1se_2 sequence can be used, in certain embodiments, for identifying CD14dimCD16+(CD115+CD11b+GR1− (Ly6C−)) monocyte functions. Monocyte functions identified can be classical or non-classical monocyte functions.

In embodiments, an immune disorder, inflammatory response, inflammation, autoimmune response, disorder or diseases is rheumatoid arthritis, juvenile rheumatoid arthritis, osteoarthritis, psoriatic arthritis, multiple sclerosis (MS), encephalomyelitis, myasthenia gravis, systemic lupus erythematosus (SLE), asthma, allergic asthma, autoimmune thyroiditis, atopic dermatitis, eczematous dermatitis, psoriasis, Sjögren's Syndrome, Crohn's disease, aphthous ulcer, iritis, conjunctivitis, keratoconjunctivitis, ulcerative colitis (UC), inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common γ chain (γc) deficiency, Janus-associated kinase 3 (JAK3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (γ5/14.1) deficiency, Hyper-IgM syndrome: X-linked (CD40 ligand deficiency) or non-X-linked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon γ receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1A/SAP deficiency), and hyper IgE syndrome.

In another aspect, there is provided a pharmaceutical composition comprising an agonist or antagonist of a transcription factor that regulates expression of Nr4a1 (Nur77) for treatment of an aberrant immune response, immune disorder, inflammatory response, inflammation, an autoimmune response, disorder or disease, cancer or an adverse cardiovascular event or cardiovascular disease in a subject. In certain embodiments, the transcription factor is Klf2 or Klf4. In some embodiments, a pharmaceutical composition comprises an agonist or antagonist of Klf2 or Klf4.

In certain embodiments, a pharmaceutical composition described herein is used to regulate expression of Nr4a1 (Nur77) for treatment of an aberrant immune response, immune disorder, inflammatory response, inflammation, an autoimmune response, disorder or disease, lymphoproliferative disorder, cancer or an adverse cardiovascular event or cardiovascular disease in a subject. In certain embodiments, a pharmaceutical composition described herein is used for the treatment of an autoimmune disease or a lymphoproliferative disease.

In embodiments, the pharmaceutical composition is for treating an individual who has a disease by administering to the individual a pharmaceutical composition including a therapeutically effective amount of a transcription factor that regulates expression of Nr4a1 (Nur77) and a pharmaceutically acceptable excipient.

The compositions disclosed herein can be administered by any means known in the art. For example, compositions may include administration to a subject intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intrathecally, subcutaneously, subconjunctival, intravesicularlly, mucosally, intrapericardially, intraumbilically, intraocularly, orally, locally, by inhalation, by injection, by infusion, by continuous infusion, by localized perfusion, via a catheter, via a lavage, in a creme, or in a lipid composition. Administration can be local or systemic.

Solutions of the active compounds as free base or pharmacologically acceptable salt can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms.

Pharmaceutical compositions can be delivered via intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal preparations are known and can include, for example, antibiotics and antihistamines.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In some embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like. The percentage of the compositions and preparations may, of course, be varied and may conveniently be between about 2 to about 75% of the weight of the unit, or typically between 25-60%. The amount of active compounds in such compositions is such that a suitable dosage can be obtained.

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion.

Sterile injectable solutions can be prepared by incorporating the active compounds or constructs in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. DMSO can be used as solvent for extremely rapid penetration, delivering high concentrations of the active agents to a small area.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

All patents, patent applications, publications, and other references, GenBank citations and ATCC citations cited herein are incorporated by reference in their entirety. In case of conflict, the specification, including definitions, will control.

All of the features disclosed herein may be combined in any combination. Each feature disclosed in the specification may be replaced by an alternative feature serving a same, equivalent, or similar purpose.

Any appropriate element disclosed in one aspect or embodiment of a method or composition disclosed herein is equally applicable to any other aspect or embodiment of a method or composition. For example, the therapeutic agents set forth in the description of the pharmaceutical compositions provided herein are equally applicable to the methods of treatment and vice versa.

As used herein, the singular forms “a”, “and,” and “the” include plural referents unless the context clearly indicates otherwise. Thus, for example, reference to “an agonist” “an antagonist” includes a plurality of such agonists and antagonists.

As used herein, all numerical values or numerical ranges include integers within such ranges and fractions of the values or the integers within ranges unless the context clearly indicates otherwise. Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-850, includes ranges of 1-20, 1-30, 1-40, 1-50, 1-60, 10-30, 10-40, 10-50, 10-60, 10-70, 10-80, 20-40, 20-50, 20-60, 20-70, 20-80, 20-90, 50-75, 50-100, 50-150, 50-200, 50-250, 100-200, 100-250, 100-300, 100-350, 100-400, 100-500, 150-250, 150-300, 150-350, 150-400, 150-450, 150-500, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed in any way.

### EXAMPLES

### Example 1

Deleting an Nr4a1 Super-Enhancer Subdomain Ablates Ly6C^{low} Monocytes while Preserving Macrophage Gene Function

Summary

Mononuclear phagocytes are a heterogeneous family that occupy all tissues and assume numerous roles to support tissue function and systemic homeostasis. Our ability to dissect the roles of individual subsets is limited by a lack of technologies that ablate gene function within specific mononuclear phagocyte sub-populations. Using Nr4a1-dependent Ly6C^{low} monocytes a proof-of-principle approach that addresses these limitations is disclosed. Combining ChIP-Seq and molecular approaches we identify a single, conserved, sub-domain within the Nr4a1 enhancer that is essential for Ly6C^{low} monocyte development. Mice lacking this enhancer lack Ly6C^{low} monocytes but retain Nr4a1 gene expression in macrophages during steady state and in response to LPS. As Nr4a1 regulates inflammatory gene expression and Ly6C^{low} monocytes, decoupling these processes allows Ly6C^{low} monocytes to be studied without confounding influences.

Introduction

Mononuclear phagocytes (MP) are an ontologically diverse family of cells comprised of macrophages and monocytes (Mo) (Perdiguero and Geissmann, 2016). From a traditional standpoint the primary functions of MP involve recognition and clearance of invading pathogens, maintaining tissue integrity and resolving inflammation (Sica and Mantovani, 2012). To perform these diverse functions MP occupy all bodily tissues and display a tremendous degree of phenotypic plasticity. In recent years transcriptomic profiling efforts have revealed the full extent of this heterogeneity, and functional studies have unveiled diverse, specialized and often unexpected roles for individual MP subsets (Amit et al., 2016). In this context, tissue macrophages and blood Mo may be considered accessory cells that allow optimal performance of the host tissue (Okabe and Medzhitov, 2016). In complement to their roles in immunity and host defense, tissue MP are now also recognized as major regulators of higher-order physiological processes including systemic energy balance (Odegaard and Chawla, 2011), intestinal peristalsis (Muller et al., 2014) and cognitive function (Parkhurst et al., 2013). Delineating the full extent of MP functions in health and disease represents a largely unexplored frontier in both immunology and physiology.

Mo constitute the blood-borne phase of the MP system and are composed of at least two subsets in both mouse and human that are believed to be conserved (Cros et al., 2010). Mouse Mo subsets can be discriminated using the Ly6C surface antigen (Geissmann et al., 2010), Ly6C^{hi} Mo are progenitors of inflammatory, and some tissue-resident macrophages. Ly6C^{low} Mo exhibit a patrolling behavior on the vascular endothelium and contribute to vascular homeostasis by maintaining the endothelial layer (Carlin et al., 2013). In addition, Ly6C^{low} Mo have recently been shown to play a crucial role protecting against the seeding of tumor metastases in the lung (Hanna et al., 2015).

An indispensable approach to understand cellular behavior is through loss-of-function. The identification of lineage defining transcription factors (LDTFs) for MP subsets has led to new insights into the functions of these cells. For example, the transcription factor Nr4a1 is the master regulator of the Ly6C^{low} Mo subset (Hanna et al., 2011). Nr4a1 is uniquely highly expressed in Ly6C^{low} Mo, and is required in a cell-intrinsic fashion. Accordingly Nr4a1^{-/-} mice were instrumental in revealing a role for Ly6C^{low} Mo in tumor metastasis (Hanna et al., 2015). In another example, loss of the transcription factor Spic selectively ablates splenic red pulp macrophages revealing a role for this population in iron homeostasis (Kohyama et al., 2009). Similarly macrophage-specific deletion of Gata6 impairs the maturation of peritoneal macrophages leading to the discovery that these cells modulate B-1 cell IgA production (Okabe and Medzhitov, 2014).

Pleiotropy, exemplified by the differential action of a single gene in multiple cell types, is a major obstacle to understanding cell-specific gene function. To overcome this problem the Cre recombinase-loxP (Cre-Lox) system is routinely used to ablate genes containing loxP-flanked exons by controlling Cre enzyme expression with cell-specific promoters. The ability of the Cre-Lox system to excise gene expression in a cell-specific manner is limited by the cell-specificity of Cre transgenes and the time taken for recombination to occur (Yona et al., 2012). These considerations present problems when using the Cre-Lox system to study gene function within populations of closely related cells. A prime example of this problem is Nr4a1. Nr4a1^{-/-} mice lack Ly6C^{low} Mo, macrophage Nr4a1 is also induced by LPS and represses inflammatory gene expression (Hanna et al., 2012). These confounding influences limit the utility of Nr4a1^{-/-} to study Ly6C^{low} Mo. Furthermore, the current MP Cre transgenes (Lys2-Cre, Csf1r-Cre, Cx3cr1-Cre) cannot delete Ly6C^{low} Mo Nr4a1 without also disrupting Nr4a1 across MP subsets. These pan-myeloid effects of the current myeloid Cre transgenes limit the ability of conditional deletion approaches to determine gene- and cell-type function within the diverse MP compartment.

Enhancers are critical determinants of gene expression (Andersson et al., 2014) and are identified by chromatin immunoprecipitation sequencing (ChIP-Seq) on the basis of high levels of histone H3 lysine 4 monomethylation (H3K4me1) and dimethylation (H3K4me2) (Heintzman et al., 2007; Heinz et al., 2010). MP enhancers are enriched for the LDTFs PU.1 and C/EBPβ, which instruct enhancer selection (Heinz et al., 2010). Enhancers are subject to additional regulation leading to their further classification as ‘poised’ or ‘active’. Enhancers are activated upon binding of signal dependent transcription factors (SDTFs), leading to acetylation at H3 lysine 27 (H3K27ac) and the increased expression of associated genes (Creyghton et al., 2010; Heinz et al., 2013). The enhancer landscapes between MP subsets show considerable diversity (Gosselin et al., 2014; Lavin et al., 2014) and result from the myriad environmental niches these populations are exposed to. As Nr4a1 shows unique expression characteristics in Ly6C^{low} Mo, we hypothesize a SDTF acting at a cell-specific enhancer regulates transcription of the Ly6C^{low} Mo LDTF Nr4a1.

Here we explore this hypothesis by mapping the Nr4a1 enhancer locus in Ly6C^{low} Mo. We identify a single sub-domain 4 kb upstream of the Nr4a1 transcription start site that is essential for Ly6C^{low} Mo development. Dissection of this element provides insight into the transcriptional processes driving Ly6C^{low} Mo development. Furthermore, using mice that lack this enhancer we show that macrophage Nr4a1 gene expression is unaffected both in response to inflammatory signaling and during steady state.

Results

Ly6C^{low} Mo are ontological neighbors to Ly6C^{hi} Mo

Lineage tracing and BrdU pulse-chase studies have established a consensus that Ly6C^{hi} Mo give rise to Ly6C^{low} Mo (Yona et al., 2012). Yet it remains possible that Ly6C^{low} Mo arise independent of the Ly6C^{hi} population. The Mo dendritic cell precursor (MDP) that expresses Flt3 (CD135), Cx3cr1 and Kit (CD117) gives rise to all mouse Mo (Hettinger et al., 2013). MDPs undergo further restriction towards the Mo lineage upon their differentiation into the common Mo progenitor (cMoP), at which point expression of FLT3 is lost and Ly6C expression is gained (Hettinger et al., 2013).

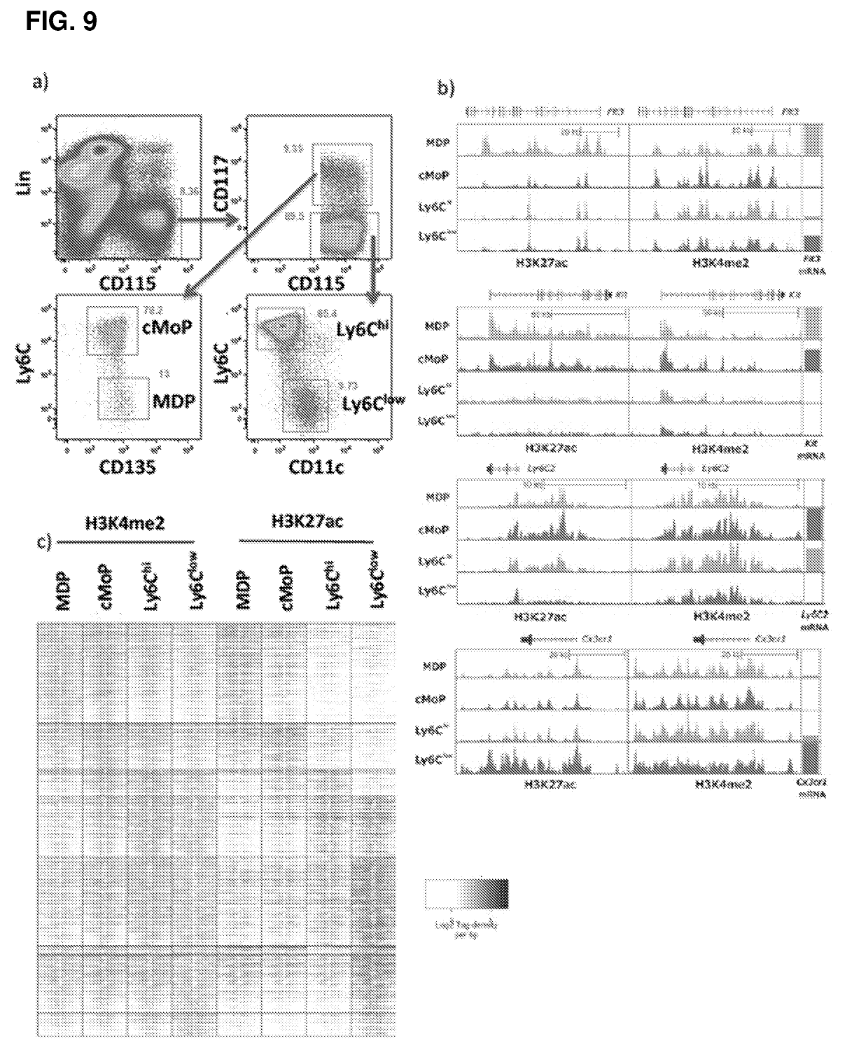

We reasoned that if Ly6C^{low} Mo arise independently of the Ly6C^{hi} population that we would observe a signature that was common to the Ly6C^{low} Mo and their progenitor, but not the Ly6C^{hi} Mo. To test this hypothesis we performed ChIP-Seq on MDP, cMoP, Ly6C^{hi} and Ly6C^{low} Mo (FIG. 9a) using H3K4me2 to define enhancers, and activity with H3K27ac. To assess data quality we visually inspected loci associated with prototypical marker genes for the profiled cell types (FIG. 9b). As expected MDPs showed enrichment for H3K27ac at the Flt3 locus whereas both MDPs and cMoPs showed H3K27ac at the Kit locus, both used to sort these populations. In contrast to Ly6C^{low} Mo, Ly6C^{hi} Mo and cMoPs show enhancer activity at the Ly6C2 locus encoding for the Ly6C surface antigen. Surprisingly MDPs also showed activity at the Ly6C2 locus in spite of low mRNA and cell-surface protein levels (FIG. 9a,b), perhaps indicating a priming of this locus prior to transcription. Finally, in Ly6C^{low} Mo Cx3cr1 shows marked H3K27ac reflecting the robust Cx3cr1 expression in this population (Carlin et al., 2013).

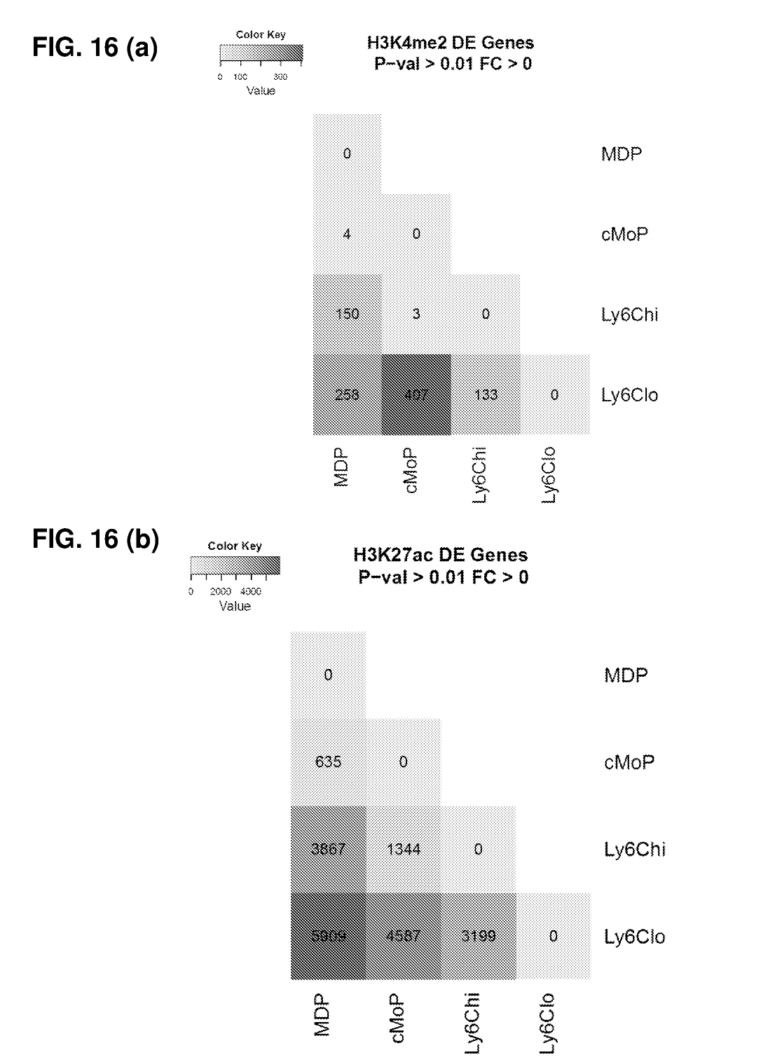

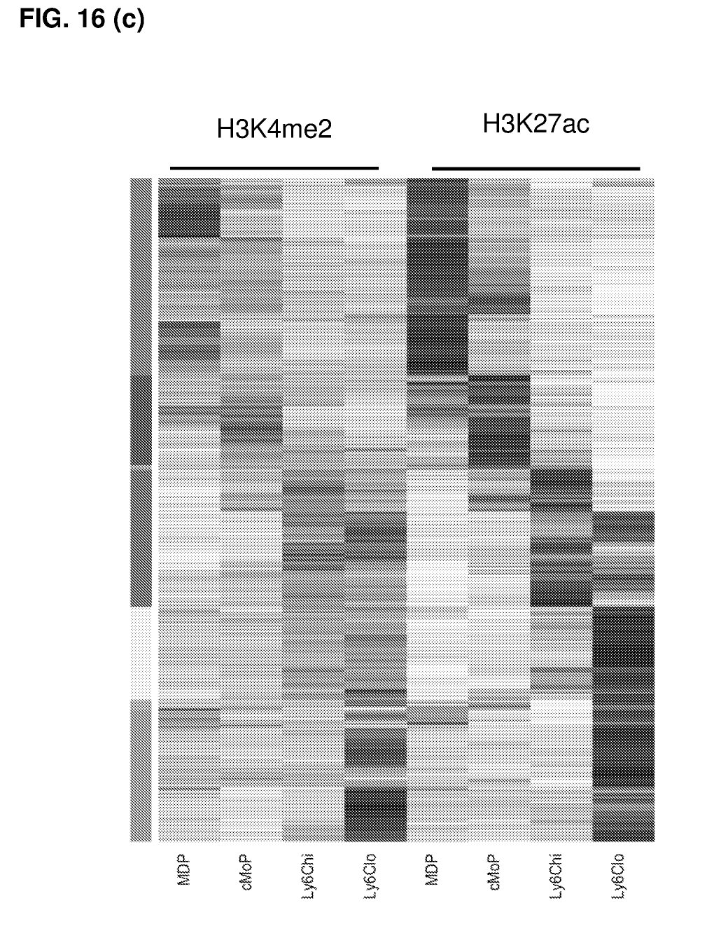

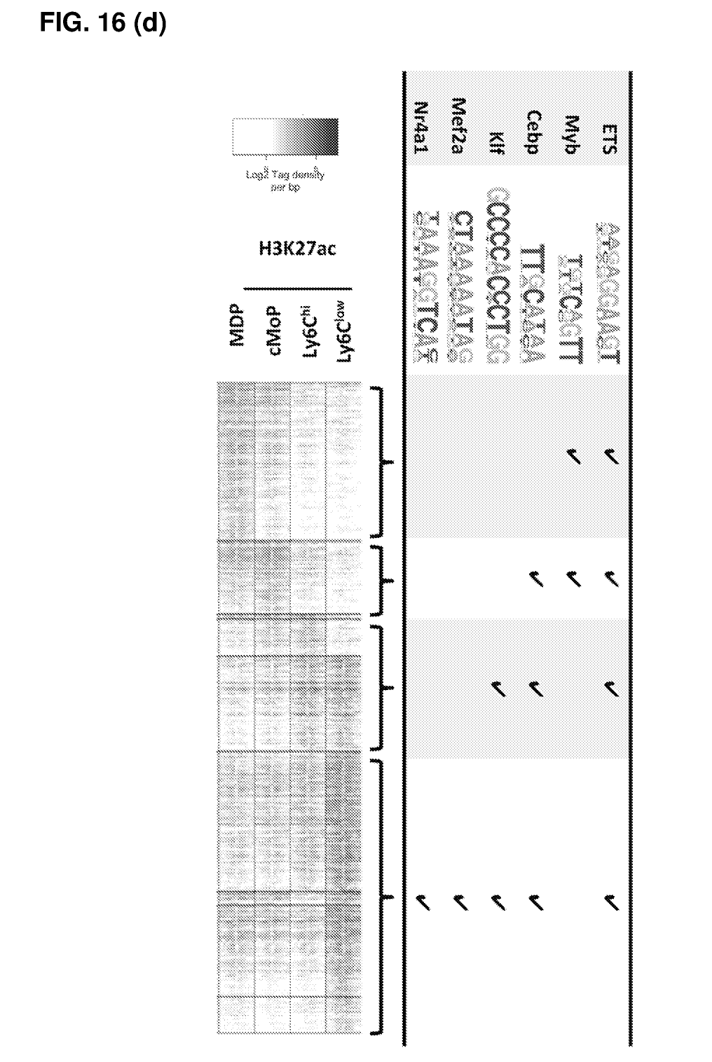

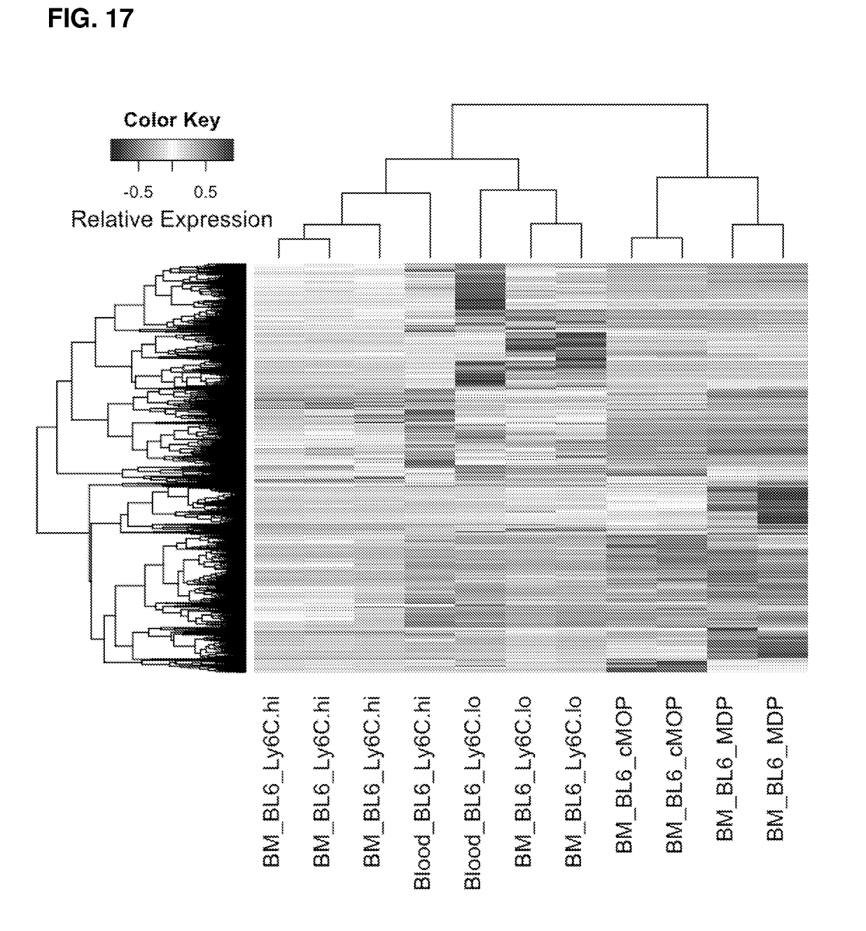

To gain a global overview of enhancer dynamics during Mo development H3K4me2 and H3K27ac were quantitated at all 99,462 enhancers defined as H3K4me2 enriched regions greater than 2.5 kb from the nearest transcription start site. Differential enrichment (DE) of histone marks was then computed between all pairwise comparisons identifying a total of 620 DE H3K4me2 regions (0.62% of total, FIG. 16(a)) and 9,879 H3K27ac regions (9.93% of total, FIG. 16(b)). Unbiased hierarchical clustering of DE enhancers (FIG. 1(c), FIG. 16(c)) showed that significant changes in H3K27ac are mirrored by more subtle shifts in H3K4me2. De novo motif analysis within these defined clusters identifies transcriptional processes driving Mo differentiation (FIG. 16(d)). All enhancer clusters were enriched for ETS (PU.1) motifs. Progenitor-associated enhancers showed restricted over-representation of Myb motifs, which were lost upon Ly6C^{hi} Mo differentiation, concomitant with the acquisition of CEBP, and KLF motifs. At the global acetylation level Ly6C^{low} Mo enhancers were uniquely characterized by over-represented NR4A and Mef2a motifs. Importantly we did not observe a signature of enhancer or motif usage that was shared between Ly6C^{low} Mo and either progenitor but not the Ly6C^{hi} Mo, nor did such a pattern emerge at the transcriptomic level (FIG. 17). Instead, the patterns of differential enhancer usage are consistent with a continuous transition between the MDP and Ly6C^{low} Mo with both the cMoP and Ly6C^{hi} Mo falling as intermediaries in this cascade. Collectively, these data strongly support the consensus model of Mo development in which Ly6C^{low} Mo derive directly from the Ly6C^{hi} Mo population.

Ly6C^{low} Mo Possess a Cell-Specific Super-Enhancer at the Nr4a1 Locus.

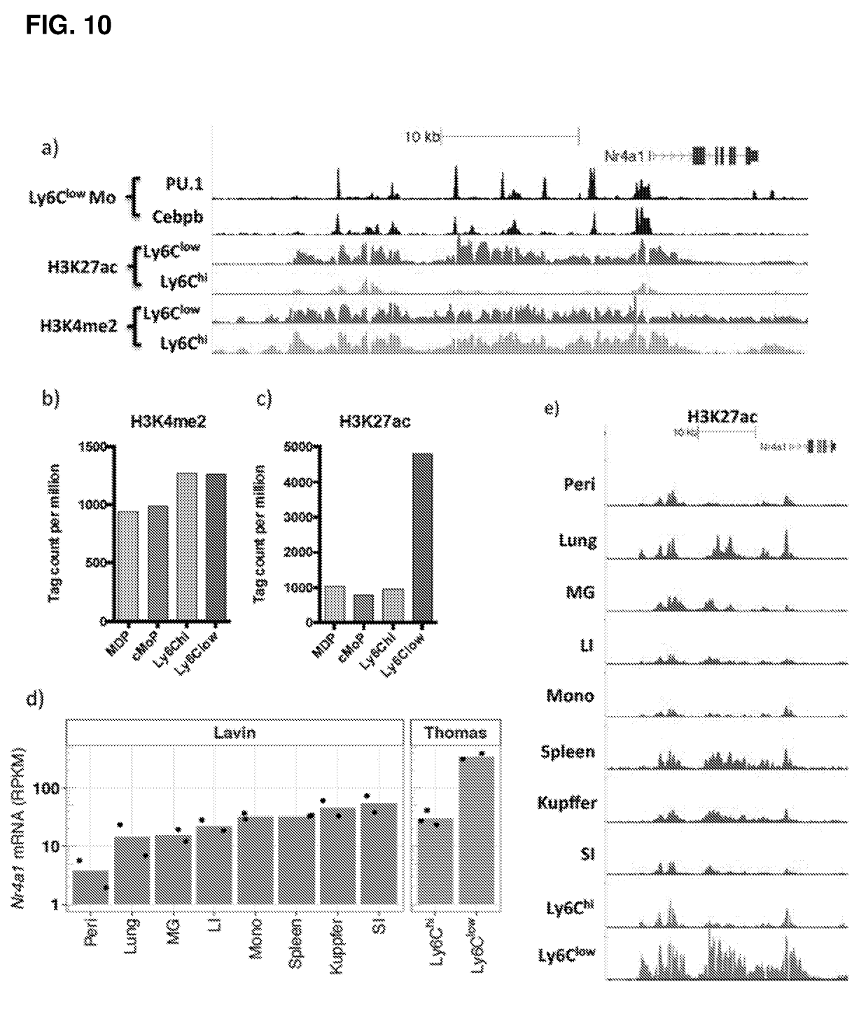

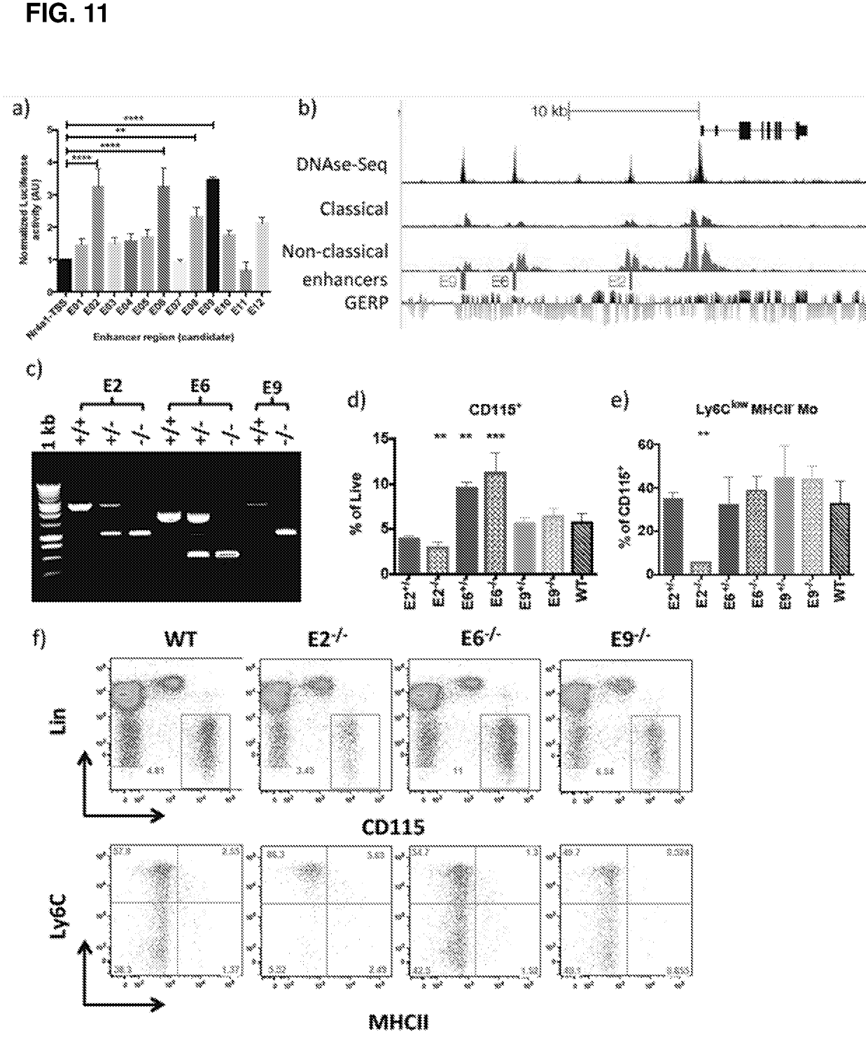

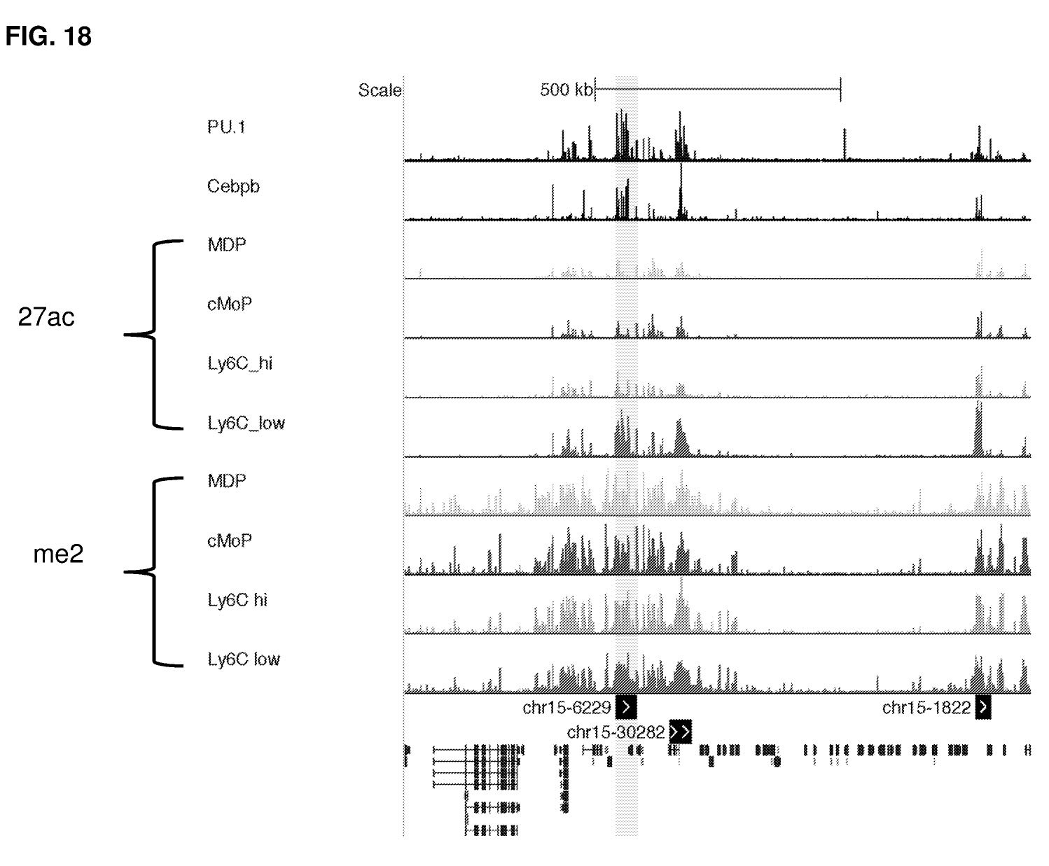

Super-enhancers (SE) are a recently defined class of highly cell-type specific enhancer (Whyte et al., 2013) that selectively mark genes involved in lineage specification and function. The defining features of SEs include their extended size, adornment with LDTFs and genomic correlates of high gene expression (Hnisz et al., 2013). As Ly6C^{low} Mo highly express Nr4a1 and require it for their development (Hanna et al., 2011) we postulated that it is a SE regulated gene. We used H3K27ac levels to define SEs in Ly6C^{low} Mo, confirming that Nr4a1 does overlap a SE (Nr4a1se; FIGS. 10(a) and 17). Validating the SE characteristics of Nr4a1se, we also observed a high density of the MP LDTFs PU.1 and CEBPβ (FIG. 18). Enumeration of H3K4me2 and H3K27ac tag counts at Nr4a1se in MDP, cMoP, Ly6C^{hi} and Ly6C^{low} Mo shows a selective and robust (5-fold) acquisition of H3K27ac in Ly6C^{low} Mo, demonstrating that Nr4a1se is a Ly6C^{low} Mo-specific SE region (FIG. 10b, c).

Formation of SEs represents a confluence of developmental and environmental cues (Hnisz et al., 2015). We leveraged the phenotypic heterogeneity of the diverse MP family to address whether steady-state Nr4a1 expression may be regulated by distinct mechanisms. First we assessed Nr4a1 expression in MP populations taken from Lavin et al (2014) and our own Mo subset data. We observe steady-state levels of Nr4a1 spanning three orders of magnitude (FIG. 10d). Importantly Nr4a1 expression in Ly6C^{hi} Mo from both datasets (Ly6C^{hi} and Mono) are consistent, facilitating the comparison between datasets. Consistent with previous observations we also observe by far the highest Nr4a1 expression in Ly6C^{low} Mo (Hanna et al., 2011).

Next we questioned whether the range of Nr4a1 mRNA expression is associated with the continuous acquisition of a fixed enhancer signature at Nr4a1se, or if Ly6C^{low} Mo possesses a unique H3K27ac profile. We visualized H3K27ac at Nr4a1se in MP populations corresponding those in FIG. 10d (FIG. 10e). We observed comparable H3K27ac tag distributions between Ly6C^{hi} Mo populations, facilitating a qualitative comparison between datasets. Each population was found to differ in both the absolute level of histone acetylation observed and, to varying degrees, also in the histone acetylation ‘fingerprint’ at Nr4a1se (FIG. 10(e)). Notably, the most intense H3K27ac signature at Nr4a1se was observed in Ly6C^{low} Mo (FIG. 10(d) & FIG. 10(e)). Taken together, these findings imply that distinct mechanisms may regulate steady-state Nr4a1 expression between MP populations.

Nr4a1Se_2 is a Conserved SE Sub-Domain Essential for Ly6C^{low} Mo Development

We sought to identify regions of Nr4a1se that regulate Nr4a1 gene expression in Ly6C^{low} Mo. Based on PU.1 and C/EBPβ binding in Ly6C^{low} Mo (FIG. 19(a)) we cloned 12 candidate sequences into luciferase reporters containing a minimal Nr4a1 promoter (FIG. 19(b). E2, E6, E8 and E9 induced modest but consistent luciferase activity in the myeloid RAW264.7 cell line indicating that these regions may regulate Nr4a1 in Ly6C^{low} Mo (FIG. 11a). It has been suggested that human CD14^{dim}CD16^{hi} Mo are homologous to mouse Ly6C^{low} Mo. This notion is based on global gene expression profiling and observation of the characteristic patrolling behavior in CD14^{dim}CD6^{hi} Mo (Cros et al., 2010). We reasoned that if CD14^{dim}CD16^{hi} and Ly6C^{low} Mo are truly orthologous that Nr4a1 should be regulated by a conserved mechanism. To test this hypothesis we assessed the genetic regulatory state of human DNA sequences orthologous to mouse E2, E6, E8 and E9 in human Mo using publicly available datasets (Boyle et al., 2008; Schmidl et al., 2014). Human Mo DNase-Seq clearly shows open chromatin at orthologous regions to mouse E2, E6 and E9 (FIG. 11b), however no orthologue for E8 was identified using BLAT. Human E2, E6 and E9 also possessed H3K27ac, and H3K27ac levels were higher at E2 and E6 in CD14^{dim}CD16^{hi} Mo than CD14^{hi}CD16^{neg} Mo, consistent with the pattern observed between mouse Mo subsets (FIG. 11b). This provides evidence that E2, E6 and E9 are functionally conserved enhancer elements between species, supporting the notion that these regions are important regulators of MP Nr4a1 gene expression.

To test the in vivo functions of E2, E6 and E9 we used the CRISPR-Cas9 system to generate three mouse strains, each containing a deletion of enhancer sequence to give Nr4a1se_2^{-/-}, Nr4a1se_6^{-/-} and Nr4a1se_9^{-/-} mice respectively (FIG. 11c, FIG. 19(c)). FACS analysis of peripheral Mo revealed a reduction in Mo frequencies in Nr4a1se_2^{-/-}, no difference in Nr4a1se_9^{-/-}, and a modest increase in Nr4a1se_6^{-/-} mice (FIG. 11d). WT-like Mo subset ratios were preserved in both Nr4a1se_6^{-/-} and Nr4a1se_9^{-/-} mice, however a striking deficit in Ly6C^{low} Mo phenocopying the Nr4a1^{-/-} strain was present in Nr4a1se_2^{-/-} mice (FIG. 11e,f) (Hanna et al., 2011). Therefore the conserved super-enhancer sub-domain Nr4a1se_2 is indispensable for Ly6C^{low} Mo development.

Nr4a1Se_2 Deletion Decouples Inflammation-Associated Nr4a1 Gene Expression from Ly6C^{low} Mo-Associated Nr4a1 Expression.

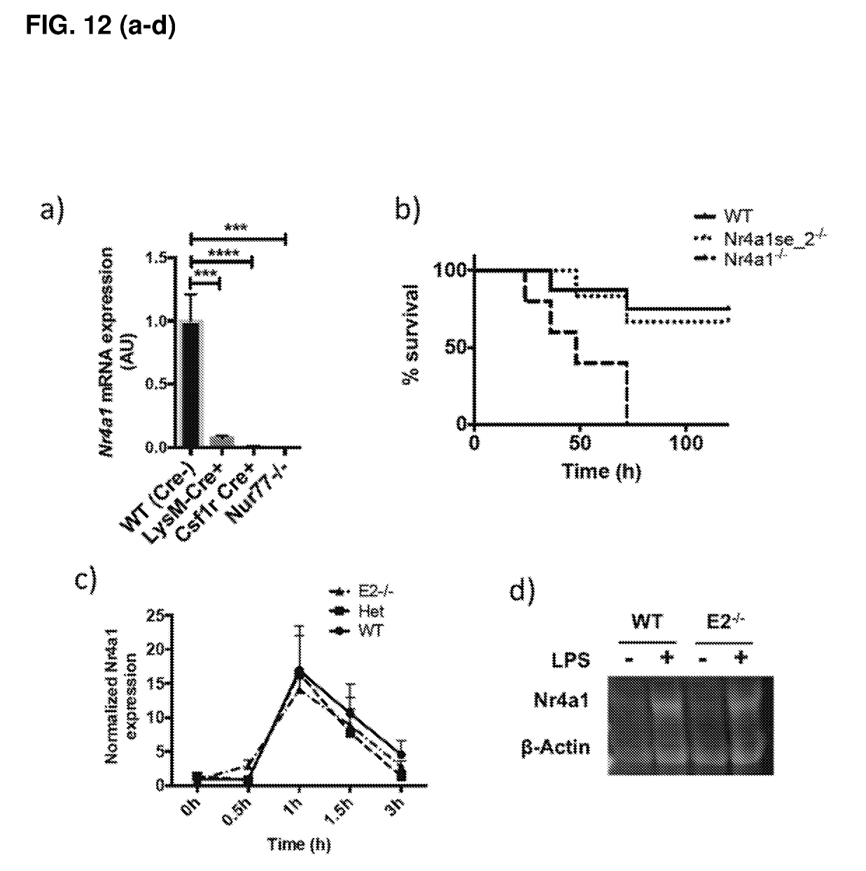

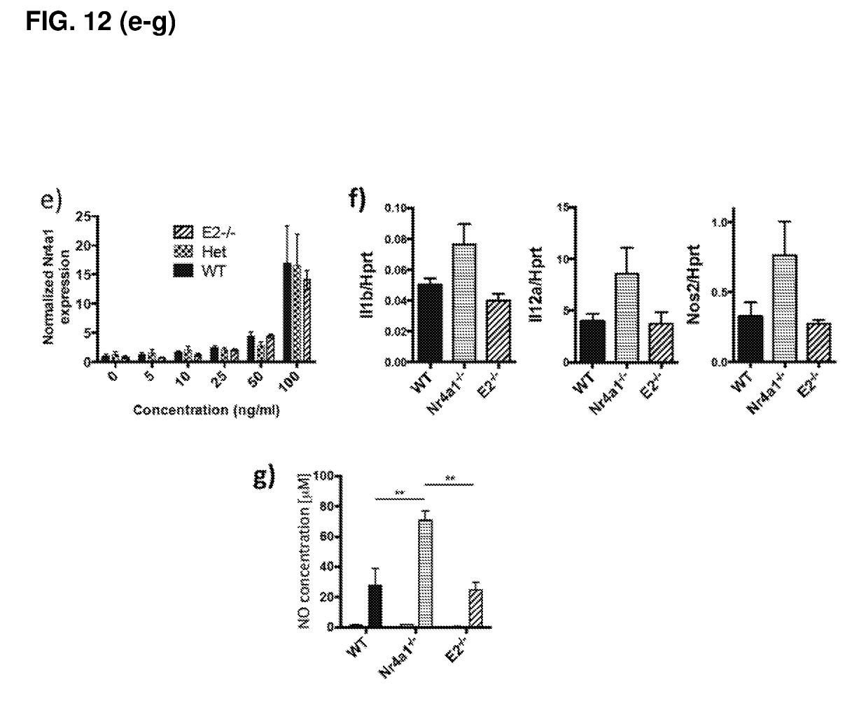

Current genetic models to study MP Nr4a1 are based on global Nr4a1^{-/-} mice or Cre recombinase mediated deletion of Nr4a1^{flox/flox} alleles using myeloid specific Cre transgenes, which include, but are not restricted to Csf1r, Lys2 (LysM) and Cx3cr1. While these tools ablate MP Nr4a1 they are non-specific with respect to individual MP subsets (Chow et al., 2011; Yona et al., 2012). Consequently, in models that lack Ly6C^{low} Mo, Nr4a1 is also disrupted in macrophages (FIG. 12a). As Ly6C^{low} Mo are absent in Nr4a1se_2^{-/-} mice and Nr4a1 is a key suppressor of macrophage inflammatory gene expression we questioned whether Nr4a1se_2^{-/-} macrophages possess wild type-like responses to inflammatory stimuli.

During endotoxic shock macrophage mediated production of inflammatory cytokines ultimately precipitates organ failure and mortality (Jacob et al., 2007). As Nr4a1^{-/-} mice are more sensitive to LPS-induced organ failure (Li et al., 2015) we decided to test the role of Nr4a1se_2 in this setting. We administered a single high dose of LPS (2.5 mg/kg) to Nr4a1se_2^{-/-}, Nr4a1^{-/-}, and WT mice by IP injection. All three groups displayed disheveled fur and shivering within the first 72 hours however Nr4a1^{-/-} mice showed a significantly higher mortality than both WT and Nr4a1se_2^{-/-} groups, whose symptoms resolved in the 72-120-hour time window (FIG. 12b). To understand the role of Nr4a1se_2 in the regulation of Nr4a1 gene expression we measured Nr4a1 responses to LPS stimulation in thioglycollate-elicited macrophages. WT, Nr4a1se_2^{-/-} and Nr4a1se_2^{-/-} macrophages all show the well-characterized peak of Nr4a1 mRNA at 1 h followed by a return to baseline by 3 h (FIG. 12c, Pei et al., 2005). Western blot analysis also confirmed protein induction in WT and Nr4a1se_2^{-/-} macrophages at 1h following stimulation (FIG. 12d). Furthermore, in a dose-response setting (FIG. 12e) we observed no differences in Nr4a1 mRNA levels between WT, Nr4a1se_2^{+/-} and Nr4a1se_2^{-/-} macrophages. Finally, following LPS challenge we detected higher levels of Il12, Il1b and Nos2 mRNA and iNOS activity in Nr4a1^{-/-} mice relative to Nr4a1se_2^{-/-} mice, which expressed levels comparable to wild type controls (FIG. 12(f) & FIG. 12(g)). These studies confirm that the Nr4a1se_2 region does not regulate Nr4a1 expression in response to TLR4 stimulation.

Differential PU.1 Binding Reveals Candidate Regulators of Ly6C^{low} Mo Gene Expression.

We sought to determine the molecular mechanisms regulating Nr4a1 expression in Ly6C^{low} Mo. Cooperative interactions between PU.1 and secondary co-factors establish and maintain macrophage enhancer repertoires (Heinz et al., 2010, 2013). Furthermore, differences in SDTF activity elicited within tissue microenvironments are responsible for the diverse range of MP enhancers observed in vivo (Gosselin et al., 2014; Lavin et al., 2014). One mechanism for enhancer acquisition involves SDTF driving the formation of latent enhancers associated with de novo H3K4me2 and PU.1 binding (Kaikkonen et al., 2013; Ostuni et al., 2013). Thus the subset of de novo PU.1 binding events provides valuable information concerning secondary SDTF identity by virtue of motif enrichment (Gosselin et al., 2014).

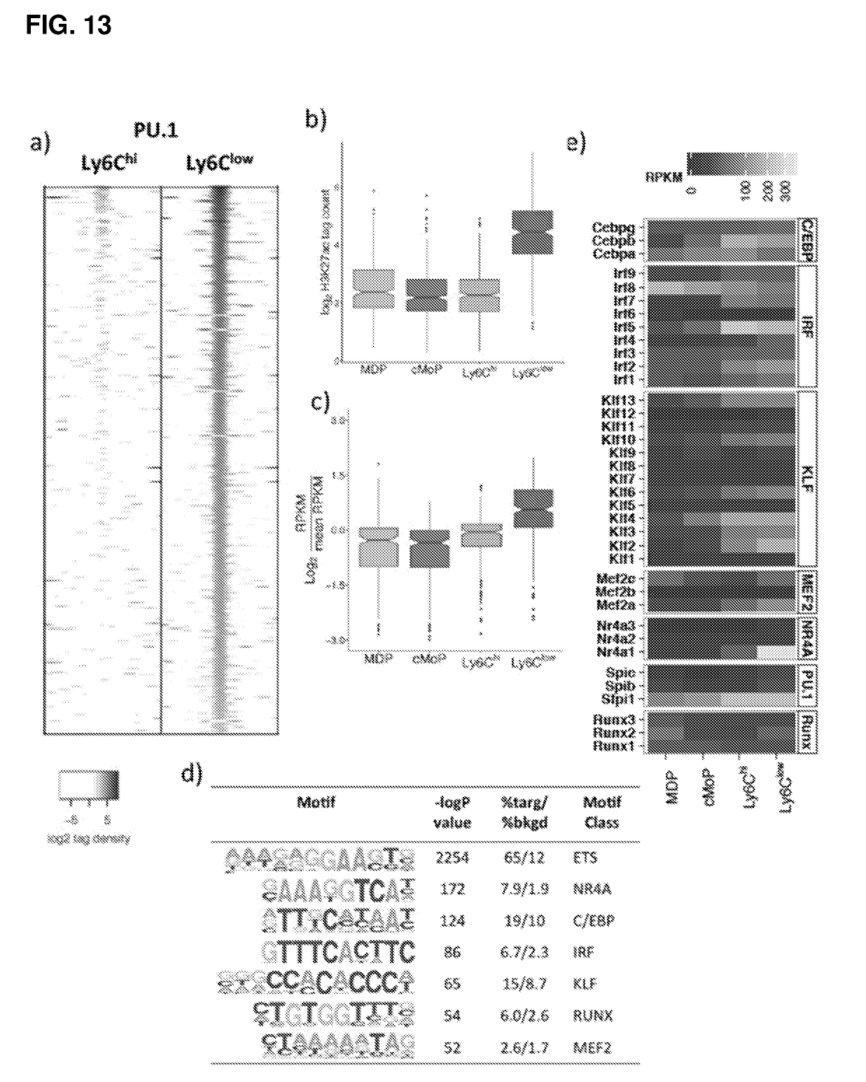

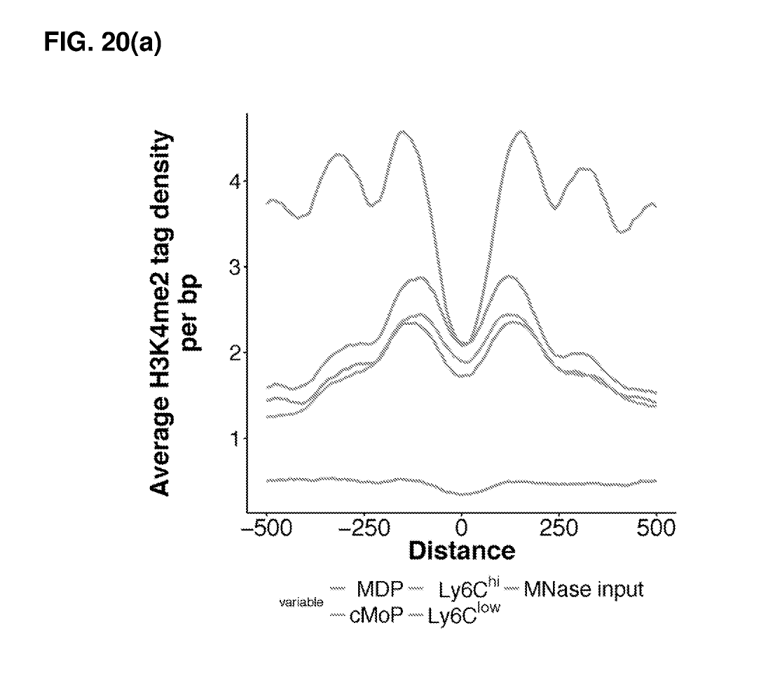

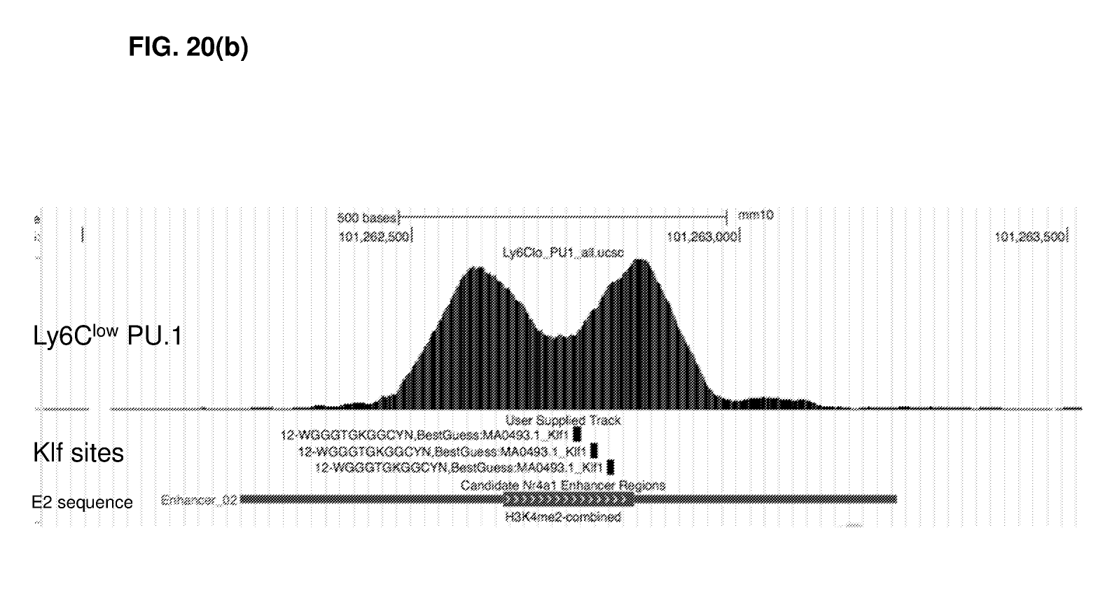

To define motifs associated with Ly6C^{low} Mo we determined PU.1 binding profiles in Ly6C^{hi} and Ly6C^{low} Mo, identifying a total of 65,070 PU.1 peaks. Strict criteria for differential binding led to the identification of 345 Ly6C^{low} Mo specific PU.1 peaks (FIG. 13a) that possess the hallmarks of a latent enhancer repertoire. These include increased H3K4me2 and nucleosome phasing at regions immediately surrounding the PU.1 peaks (FIG. 20(a)); elevated H3K27ac (FIG. 13b); and, increased expression of proximal mRNA transcripts (FIG. 13c) in Ly6C^{low} Mo relative to upstream progenitors. Thus, the Ly6C^{low} Mo PU.1-specific peak profile is associated with enhancers that regulate the Ly6C^{low} Mo gene expression program. Motifs implicated in Ly6C^{low} Mo gene expression were identified using de novo motif enrichment analysis on this PU.1 peak set. In strong agreement with our earlier analysis (FIG. 16(c)) we recovered ETS, C/EBP and NR4A motifs (FIG. 13d) as expected, alongside IRF, KLF, RUNX and MEF2 motifs. Using our RNA-Seq dataset we identified transcription factors that are expressed in Ly6C^{low} Mo and can bind to these motifs (FIG. 13(e)), considering the most abundant as our primary candidate. This approach defined Cebpb, Irf5, Klf2, Mef2a, Nr4a1, Sfpi1 (PU.1) and Runx2 as principal regulators of Ly6C^{low} Mo gene expression.

Klf2 Regulates Ly6C^{low} Mo Conversion Via Nr4a1Se_2.

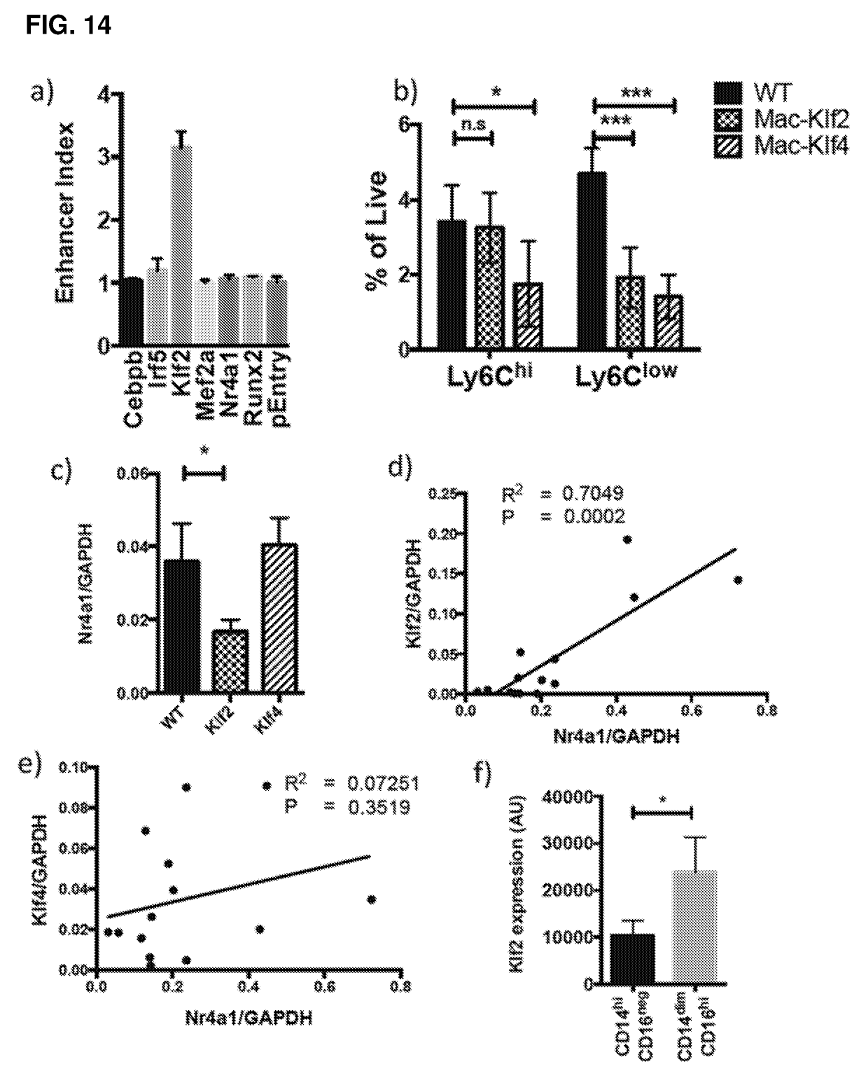

To identify transcription factors that regulate Nr4a1 expression via Nr4a1se_2 we overexpressed each candidate with the Nr4a1se_2 reporter. Cebpb, Irf5, Kf2, Mef2a, Nr4a1 and Runx2 were co-transfected into RAW264.7 macrophages alongside either the Nr4a1-TSS or Nr4a1se_2-TSS luciferase reporter plasmids. To account for promoter dependent effects of the cDNA and the intrinsic activity of the E2 sequence, an enhancer index was calculated that denotes the difference in the ratio of induced luciferase activity between Nr4a1-E2-TSS and Nr4a1-TSS. We found that only Klf2 drives E2-dependent luciferase expression (FIG. 14a). Supporting this finding a motif search for the enriched IRF, KLF, RUNX and MEF2 motifs within the E2 region identified a cluster of 3 KLF motifs but failed to find any instances of the other motif classes (FIG. 20(b)).

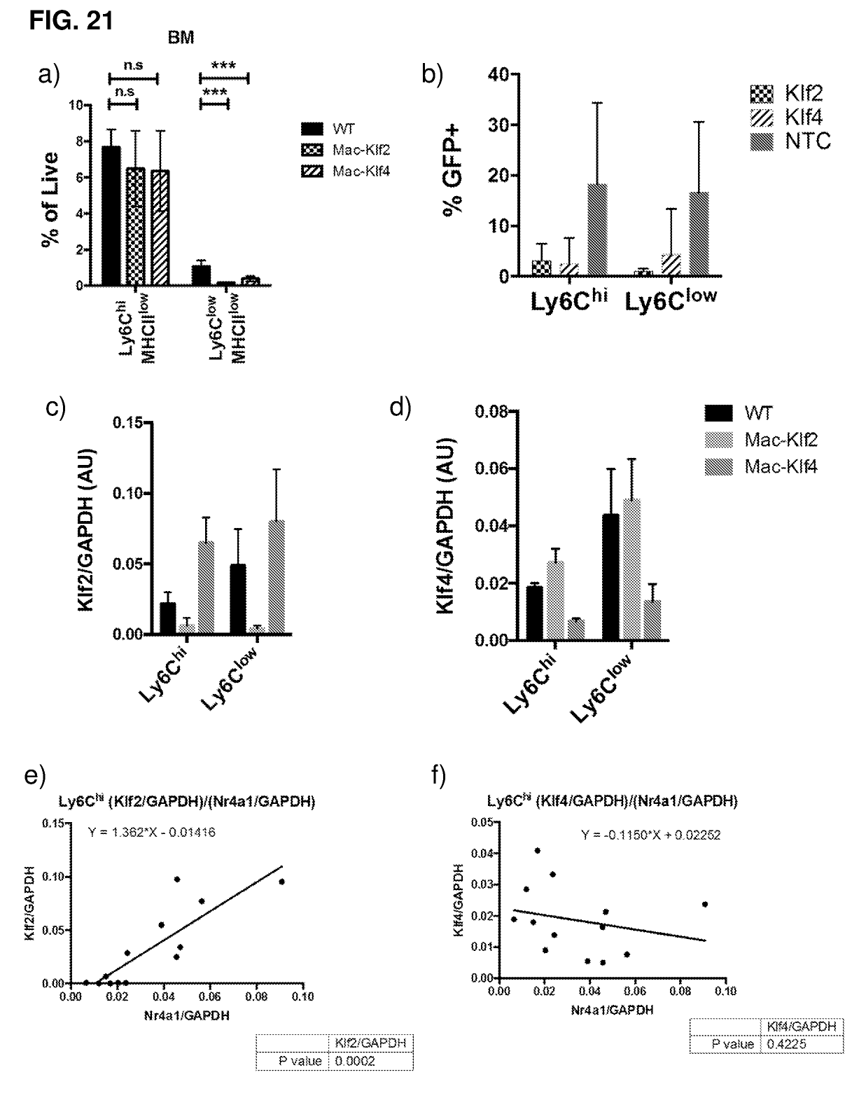

We chose to investigate the role of KLF factors in Ly6C^{low} Mo development. KLFs are broadly expressed in Ly6C^{low} Mo with Klf2 and Klf4 being the most abundant (FIG. 13e). Owing to the perinatal and embryonic lethality of Klf2^{-/-} and Klf4^{-/-} mice we assessed Mo frequencies in myeloid-specific Lyz2-Cre Klf2^{flox/flox} and Lyz2-Cre Klf4^{flox/flox} mice (denoted Mac-Klf2 and Mac-Klf4 respectively, Liao et al., 2011; Mahabeleshwar et al., 2011). Flow cytometric analysis of Mac-Klf4 blood Mo showed a decrease in both populations (FIG. 14b), consistent with previous reports (Alder et al., 2008). In Mac-Klf2 mice Ly6C^{hi} Mo were unaffected however Ly6C^{low} Mo were partially reduced (FIG. 14b). Similar results were also observed in the bone marrow of Mac-Klf2 and Mac-Klf4 mice and in bone marrow chimeric mice retrovirally transduced with shRNA targeting Klf2 and Klf4 (FIG. 21 (a) & (b)).

We have previously observed incomplete deletion of loxP-flanked genes in blood Mo using the Lys2-Cre system (Hanna et al., 2015). Therefore the differences in Mo frequencies between Mac-Klf2 and Mac-Klf4 mice may either reflect intrinsic differences in the ability of each gene to regulate Mo development, or simply the abundance of each TF in each subset. To address this we measured Klf2 and Klf4 mRNA in Ly6C^{hi} and Ly6C^{low} Mo sorted from Mac-Klf2 and Mac-Klf4 mice using primers that span the loxP-flanked exon to determine recombination efficiency. In Mac-Klf2 mice 72% and 91% loss of Klf2 mRNA was observed for Ly6C^{hi} and Ly6C^{low} Mo respectively (FIG. 21(c)). Thus the selective loss of Ly6C^{low} Mo in Mac-Klf2 mice is not consistent with the incomplete deletion of Klf2 in Ly6C^{hi} Mo. A role for Klf2 in Ly6C^{hi} to Ly6C^{low} Mo conversion is also consistent with the induction of Klf2 gene expression in the transition between subsets (FIG. 13e). In Mac-Klf4 mice Klf4 mRNA was reduced by 63% and 69% in Ly6C^{hi} and Ly6C^{low} Mo respectively (FIG. 21(d)). Although Mac-Klf4 mice possess fewer Ly6C^{low} Mo it is not clear whether this results from fewer precursor Ly6C^{hi} Mo, a decrease in Ly6C^{hi} to Ly6C^{low} Mo conversion, or both. We reasoned that if Klf2 or Klf4 regulate Ly6C^{hi} to Ly6C^{low} Mo conversion via Nr4a1 that Klf2/4 gene expression would predict Mo Nr4a1 expression. Indeed, Nr4a1 mRNA expression levels were lower in Ly6C^{hi} Mo derived from Mac-Klf2 mice, but not Mac-Klf4 mice (FIG. 14c). Subsequent investigation revealed a significant positive correlation between Klf2 and Nr4a1 transcript levels in both Mo subsets, however no such relationship was found between Klf4 and Nr4a1 gene expression (FIG. 14(d) & (e) and FIG. 21 (e) & (f)).

While of a preliminary nature, these correlative findings suggest that Klf2 regulates Ly6C^{low} Mo development via Nr4a1se_2. Given our evidence for a conserved mechanism of Nr4a1 gene expression between (FIG. 11b) we predict that KLF2 expression follows a similar pattern in human Mo subsets. Interrogation of microarray data for KLF2 in human Mo subsets confirmed this hypothesis, showing significantly higher KLF2 levels in human CD14^{dim}CD16^{hi} Mo (FIG. 14f).

Nr4a1Se_2 is a Mo-Specific Enhancer.

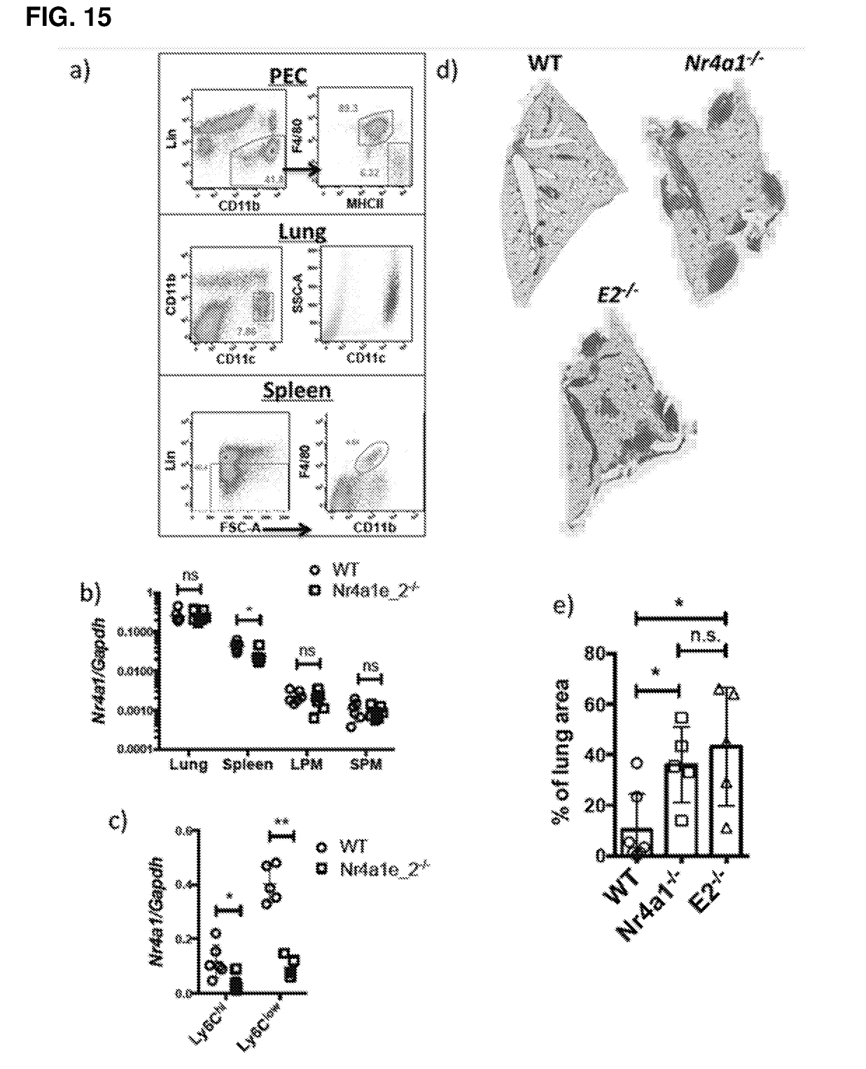

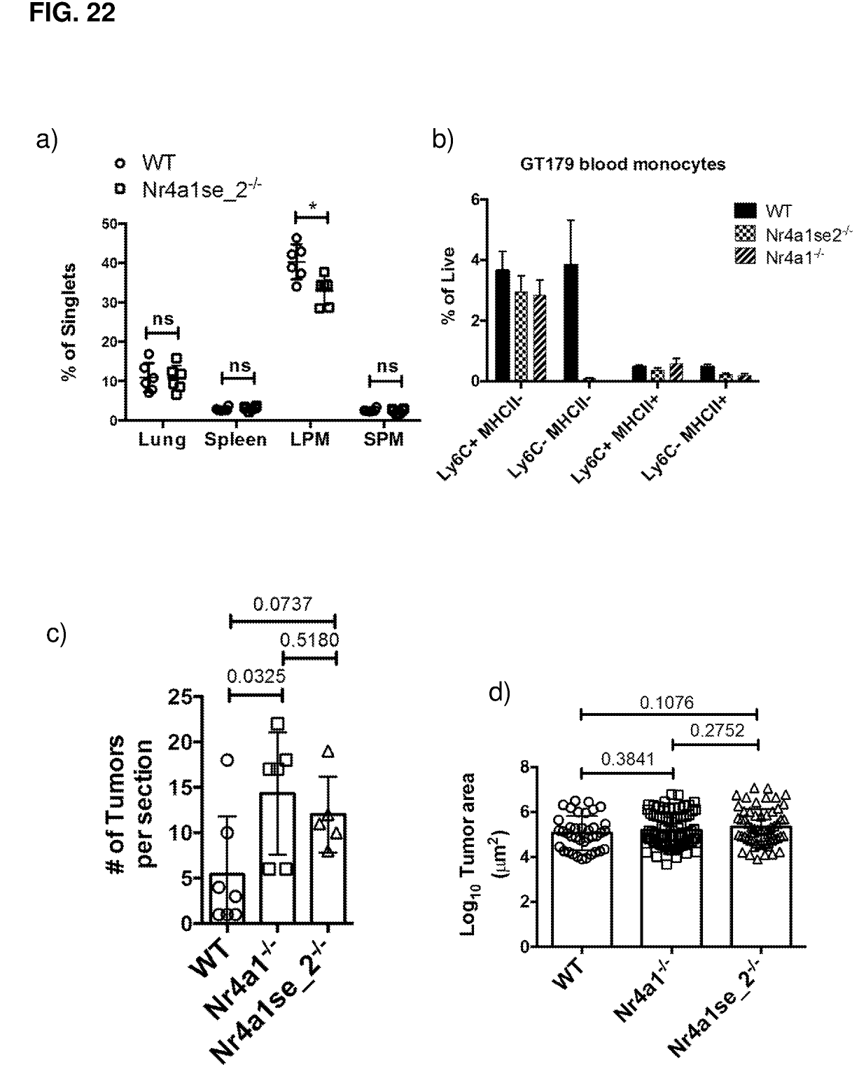

Nr4a1se_2 regulates Nr4a1 expression in Ly6C^{low} Mo but not in response to LPS stimulation. Furthermore, at steady state, macrophage Nr4a1 expression spans three orders of magnitude. To determine the extent to which Nr4a1se_2 is Ly6C^{low} Mo specific we measured Nr4a1 levels in various MP populations from Nr4a1se_2^{-/-} mice. Blood Mo, F4/80^{hi} large, F4/80^{int} small peritoneal macrophages (LPM and SPM respectively), CD11c⁺ lung alveolar macrophages, and F4/80⁺ splenic red pulp macrophages were selected (FIG. 15a). In line with previous observations (Tacke et al., 2015) macrophage frequencies were largely unchanged in Nr4a1^{-/-} mice, except for moderately fewer F4/80^{hi} LPM (FIG. 22(a)). RT-PCR analysis captured the expected range of Nr4a1 levels between macrophage populations (FIG. 15b). Strikingly however no differences in Nr4a1 mRNA expression were detected between tissue macrophages, except for moderately lower levels in Nr4a1se_2^{-/-} splenic macrophages. In contrast to this a substantial reduction in steady state Nr4a1 mRNA expression levels was observed in both Ly6C^{hi} and Ly6C^{low} Mo (FIG. 15c) showing that Nr4a1se_2 is a Mo-specific enhancer.

We recently reported that Ly6C^{low} Mo prevent cancer metastasis to the lung (Hanna et al., 2015). To demonstrate the utility of the Nr4a1se_2^{-/-} model to study Ly6C^{low} Mo we assessed tumor burden in mice using a well-established model of metastasis. B16F10 melanoma cells were intravenously injected into WT, Nr4a1^{-/-} and Nr4a1se_2^{-/-} mice. 18 days after challenge mice were sacrificed and tumor burden was measured by histology. Both Nr4a1^{-/-} and Nr4a1se_2^{-/-} mice showed a loss of Ly6C^{low} Mo (FIG. 22(b)) and significantly higher tumor burden than WT (FIG. 15d,e) however no difference was observed between Nr4a1^{-/-} and Nr4a1se_2^{-/-} mice. Consistent with a critical role for Ly6C^{low} Mo in controlling metastasis, the differences are explained by fewer tumor regions rather than tumor size (FIG. 22(c) & (d)). Therefore the Nr4a1se_2^{-/-} model effectively decouples Nr4a1-dependent inflammatory phenotypes from Ly6C^{low} Mo function, providing a new and improved tool to study the function of these cells. In contrast to conditional deletion strategies using the Cre-Lox system, enhancer targeting confers an unprecedented degree of specificity, enabling the causal inference of individual MP subsets in disease.

Discussion

### A Single Super-Enhancer Sub-Domain Regulates Ly6C^{low} Mo Development

During our investigation into the transcriptional regulatory pathways of Ly6C^{low} Mo development we identified Nr4a1se_2 as a single domain that is absolutely required for Ly6C^{low} Mo development. We also observed distinct, cell-subset specific, patterns of H3K27ac at the Nr4a1se locus leading us to investigate whether Nr4a1 gene expression is controlled by distinct mechanisms between MP subsets. Nr4a1se_2^{-/-} mice lack Ly6C^{low} Mo, yet macrophage Nr4a1 expression is largely unaffected during steady state and during inflammation. This implies a modular structure for the Nr4a1 locus control region Nr4a1se and is consistent with a recent analysis of several SEs showing that these regions consist of multiple sub-domains (Hnisz et al., 2015). Disrupting these sub-domains differentially affected target gene expression depending upon the locus. For example at SIK1, additive effects between sub-domains contribute towards the stable induction of gene expression, whereas at Prdm14, mRNA expression was regulated almost entirely by one of five sub-domains (Hnisz et al., 2015). Our study has identified three active and conserved MP enhancers upstream of Nr4a1; Nr4a1se_2, Nr4a1se_6 and Nr4a1se_9, yet only Nr4a1se_2 regulates Ly6C^{low} Mo development. This highlights the importance of detailed molecular analyses to establish the relevance of individual enhancer elements, and TF binding motifs within these elements. Whether Nr4a1se_6 and Nr4a1se_9 regulate MP Nr4a1 gene expression in response to other stimuli is a line of future enquiry.

Enhancer Targeting as a Strategy to Identify Regulators of Tissue MP Development.

Our studies suggest that KLF transcription factors regulate Nr4a1 gene expression via Nr4a1se_2. Ly6C^{low} Mo enhancers are enriched for KLF motifs, and these motifs are present in Nr4a1se_2. Multiple KLF family members are expressed in Ly6C^{low} Mo, which may act redundantly at Nr4a1se_2. In Ly6C^{low} Mo Klf2 and Klf4 are the most abundant family members, thus we investigated the roles of these genes. Both Klf2 and Klf4 regulate macrophage inflammatory gene expression by inhibiting p300 and PCAF recruitment to Nf-KB (Das et al., 2006; Liao et al., 2011). The different roles ascribed to each factor may arise from the different contexts in which they are expressed. Klf4 is induced during Ly6C^{hi} Mo differentiation, and is further up-regulated in macrophages by IL-4. In this context Klf4 regulates Ly6C^{hi} Mo development and facilitates the M2 program of macrophage activation (Feinberg et al., 2007; Liao et al., 2011). Klf2 however is expressed in circulating Mo, and strongly reduced by hypoxia leading to increased Nf-κB/HiF-1 activity (Mahabeleshwar et al., 2011).

Our findings suggest non-redundant roles for Klf2 and Klf4 in Mo development. Klf4 expression is high in both Mo subsets and Mac-Klf4 mice have fewer Mo. However, the ratio of Ly6C^{hi} to Ly6C^{low} Mo, and Ly6C^{low} Mo Nr4a1 expression are not affected in Mac-Klf4 mice (FIG. 14c). However, depletion of Klf2 facilitated a selective loss of Ly6C^{low} Mo and Klf2 mRNA levels were predictive of Nr4a1 levels, implying a causal relationship between Kf2 and Nr4a1. Despite repeated attempts we were not able to obtain successful immunoprecipitation of KLF2 at the Nr4a1se locus due to an absence of high-quality commercial antibodies, neither was overexpression of Kf2 sufficient to up-regulate Nr4a1 mRNA in vitro (data not shown). These preliminary findings suggest that Klf2 acts via Nr4a1se_2 locus and is necessary but not sufficient for Ly6C^{low} Mo development. The identification of additional critical co-factors acting via Nr4a1se_2, the Nr4a1 promoter or elsewhere will pave the way for a more detailed mechanistic understanding of how Nr4a1se_2 regulates Nr4a1 gene expression in Ly6C^{low} Mo development.

Enhancer Targeting as an Approach to Overcome Pleiotropic Effects in Closely Related Cell Types.

MP occupy all tissues of the body where these cells perform specialized functions to facilitate homeostasis. For example, Ly6C^{low} Mo maintain vascular integrity by facilitating removal of damaged endothelium (Carlin et al., 2013), alveolar macrophages maintain lung function by clearing surfactants (Nakamura et al., 2013), red pulp macrophages contribute to iron homeostasis by recycling erythrocytes (Kohyama et al., 2009), whereas the microglia's dedicated functions include supporting brain function by synaptic pruning (Paolicelli et al., 2011). These specialized functions are imparted by tissue-specific LTDFs; Ly6C^{low} Mo require Nr4a1 (Hanna et al., 2011); alveolar macrophages rely upon PPARγ (Schneider et al., 2014); splenic red pulp-macrophages macrophages need Spic (Kohyama et al., 2009); while resident peritoneal macrophages depend upon the transcription factor GATA6 (Okabe and Medzhitov, 2014).

In some cases, such as for Gata6 and Spic, LDTF expression within the MP system is cell-specific such that macrophage-specific deletion of the factor is sufficient to study the function of the associated subset. However, for the most part both the expression and requirement of key MP TFs are not cell-specific. Epigenetic analyses of MP subsets reveals shared motif usage and co-expression of cognate TFs over multiple subsets (Lavin et al., 2014). For example PPAR motifs are enriched in alveolar and renal macrophage enhancers and PPARγ regulates both alveolar macrophage and osteoclast differentiation (Schneider et al., 2014; Wan et al., 2007). Disease processes complicate matters further by invoking dynamic transcription factor expression and altering requirements, in this context PPARγ is up-regulated by IL-4 and controls alternative macrophage activation (Odegaard et al., 2007). Similarly, Nr4a1 is required for Ly6C^{low} Mo development and is also induced by LPS (Hanna et al., 2011; Pei et al., 2005). These pleiotropic effects limit our ability to study individual MP subsets and the lack of specificity provided by current myeloid Cre transgenes presents a significant problem when attempting to target individual subsets.

We have shown that enhancer targeting can overcome these limitations. Our approach leverages the unique enhancer repertoires resulting from the exclusive environment each MP subset occupies. While the concept of cell specific enhancers controlling LDTF expression is not new (Zheng et al., 2010), to our knowledge this application and degree of specificity is. We show that targeting cell-specific SE sub-domains within key LDTFs functionally decouples cell-specific aspects of gene expression while retaining physiological characteristics of gene function relevant to neighboring cell subsets. In our case, targeting the Nr4a1se_2 sub-domain has led to the generation of a new loss-of-function tool to study Ly6C^{low} Mo that preserves both Nr4a1 expression in other MP populations and the rapid kinetics of Nr4a1 expression in response to LPS signaling. Conceptually this strategy can be applied to any scenario in which gene expression is regulated by distinct enhancer sequences providing a new approach to study gene function amongst closely related cell types.

Experimental Procedures

### Mice

Mice were maintained in-house or purchased from the Jackson laboratory. All experiments followed guidelines of the La Jolla Institute for Allergy and Immunology (LIAI) Animal Care and Use Committee, and approval for use of rodents was obtained from LIAI according to criteria outlined in the Guide for the Care and Use of Laboratory Animals from the National Institutes of Health. Mice were euthanized by CO2 inhalation

### Cell Sorting

Mice were sacrificed by CO₂ inhalation and bone marrow or blood extracted into DPBS+2 mM ETDA. Following RBC lysis cells were blocked and stained for surface antigens and purified by flow cytometry as described herein.

### Thioglycollate Elicited Macrophages

Thioglycollate elicited macrophages were elicited by intraperitoneal injection of Iml 4% Brewer's Thioglycollate Medium. 5 days after injection macrophages were harvested and cultured as described in the extended experimental procedures

### RNA Isolation and RNA-Seq

Sorted cells were immediately spun down and stored in Trizol. RNA was extracted using DirectZol columns (Zymo Research). RNA was prepared for sequencing using the TruSeq v2 kit (Illumina).

### ChIP-Seq

Histone ChIP was performed using the protocol described in (Gilfillan et al., 2012) and transcription factor ChIP performed essentially as described in (Gosselin et al., 2014), with modifications described in the extended methods. ChIP-Seq libraries were prepared using the ThruPlex-FD kit (Rubicon Genomics).

### Molecular Cloning and Overexpression Studies

Enhancers were cloned into the SalI and BamH1 sites of a pGL4.10 series vector described in (Heinz et al., 2013) modified to contain an Nr4a1 minimal promoter (300 bp) and 5′UTR. Transfection assays were performed in murine RAW264.7 macrophages using Lipofectamine LTX. cDNA expression vectors were obtained from Origene. Full details are in the extended experimental procedures.

### CRISPR Mouse Generation

Mouse genome editing was performed essentially as described with minor modifications (Concepcion et al., 2015; Wang et al., 2013). Four sgRNA (two pairs targeting each enhancer flanking region) were injected into embryos of superovulated C57BL/6 mice along with Cas9 mRNA (Life Technologies) at the UCSD transgenic core facility. Knockout mice were mated with C57BL/6 and offspring thereafter maintained via sibling mating.

### Bone Marrow Chimeras

Bone marrow was shipped on ice as described in the extended experimental methods. Recipient mice were lethally irradiated and bone marrow transplanted by retroorbital injection. Mice were allowed to recover for at least 6 weeks before performing experiments.

### Cancer Study

300,000 B16F10 melanoma cells were injected via tail vein into recipient mice. Lungs were harvested in zinc buffered formalin and mounted into paraffin blocks. Sections were stained with H&E and slides were scanned with an AxioScan Z1 (Zeiss), see extended experimental procedures.

### Endotoxin Sensitivity Assay

Ultrapure LPS-EB (Invivogen) was made up in PBS and injected IP. Mice were monitored three times daily for the first 72 hours and twice daily thereafter.

### Bioinformatics Data Analysis

FASTQ files were mapped to the mouse mm10 reference genome using RNA-STAR for RNA-Seq experiments, or Bowtie for ChIP-Seq studies. RNA-Seq expression levels were quantitated using featureCounts and differential expression analyzed using edgeR. ChIP-Seq analysis was performed using HOMER. For full details see extended experimental procedures.

### Accession Numbers

Illumina sequencing for this project has been deposited at NCBI's Gene Expression Omnibus (GEO) as a SuperSeries under the accession number GSE80040.

### Acknowledgements

Funding sources: R01HLx18765, R01CA202987 and R01HL112039 to CCH; R01DK091183, R01CA17390 and R01HL088093 to CKG; R00HL123485 to CER; R01GM086912 to BAH; R01HL086548 and R01075427 to MKJ. AHA Fellowships 16POST27630002 to GDT and 12DG12070005 to RNH. KDR was supported in part by a Ruth L. Kirschstein National Research Service Award (NRSA) Institutional Predoctoral Training Grant, T32 GM008666, from the National Institute of General Medical Sciences.

### REFERENCES

Alder, J. K., Georgantas, R. W., Hildreth, R. L., Kaplan, I. M., Morisot, S., Yu, X., McDevitt, M., and Civin, C. I. (2008). Kruppel-Like Factor 4 Is Essential for Inflammatory Monocyte Differentiation In Vivo. I. Immunol. 180, 5645-5652.
Amit, I., Winter, D. R., and Jung, S. (2016). The role of the local environment and epigenetics in shaping macrophage identity and their effect on tissue homeostasis. Nat. Immunol. 17, 18-25.
Andersson, R., Gebhard, C., Miguel-Escalada, I., Hoof, I., Bornholdt, J., Boyd, M., Chen, Y., Zhao, X., Schmidl, C., Suzuki, T., et al. (2014). An atlas of active enhancers across human cell types and tissues. Nature 507, 455-461.
Boyle, A. P., Davis, S., Shulha, H. P., Meltzer, P., Margulies, E. H., Weng, Z., Furey, T. S., and Crawford, G. E. (2008). High-resolution mapping and characterization of open chromatin across the genome. Cell 132, 311-322.
Carlin, L. M., Stamatiades, E. G., Auffray, C., Hanna, R. N., Glover, L., Vizcay-Barrena, G., Hedrick, C. C., Cook, H. T., Diebold, S., and Geissmann, F. (2013). Nr4a1-Dependent Ly6Clow Monocytes Monitor Endothelial Cells and Orchestrate Their Disposal. Cell 153, 362-375.
Chow, A., Brown, B. D., and Merad, M. (2011). Studying the mononuclear phagocyte system in the molecular age. Nat. Rev. Immunol. 11, 788-798.
Concepcion, D., Ross, K. D., Hutt, K. R., Yeo, G. W., and Hamilton, B. A. (2015). Nxfl natural variant E610G is a semi-dominant suppressor of IAP-induced RNA processing defects. PLoS Genet. 11, e1005123.
Creyghton, M. P., Cheng, A. W., Welstead, G. G., Kooistra, T., Carey, B. W., Steine, E. J., Hanna, J., Lodato, M. A., Frampton, G. M., Sharp, P A., et al. (2010). Histone H3K27ac separates active from poised enhancers and predicts developmental state. Proc. Natl. Acad. Sci.
Cros, J., Cagnard, N., Woollard, K., Patey, N., Zhang, S.-Y., Senechal, B., Puel, A., Biswas, S. K., Moshous, D., Picard, C., et al. (2010). Human CD14dim monocytes patrol and sense nucleic acids and viruses via TLR7 and TLR8 receptors. Immunity 33, 375-386.
Das, H., Kumar, A., Lin, Z., Patino, W. D., Hwang, P. M., Feinberg, M. W., Majumder, P. K., and lain, M. K. (2006). Kruppel-like factor 2 (KLF2) regulates proinflammatory activation of monocytes. Proc. Natl. Acad. Sci. U.S.A. 103, 6653-6658.
Feinberg, M. W., Wara, A. K., Cao, Z., Lebedeva, M. A., Rosenbauer, F., Iwasaki, H., Hirai, H., Katz, J. P., Haspel, R. L., Gray, S., et al. (2007). The Kruppel-like factor KLF4 is a critical regulator of monocyte differentiation. EMBO J. 26, 4138-4148.
Geissmann, F., Manz, M. G., Jung, S., Sieweke, M. H., Merad, M., and Ley, K. (2010). Development of monocytes, macrophages, and dendritic cells. Science 327, 656-661.
Gilfillan, G. D., Hughes, T., Sheng, Y., Hjorthaug, H. S., Straub, T., Gervin, K., Harris, J. R., Undlien, D. E., and Lyle, R. (2012). Limitations and possibilities of low cell number ChIP-seq. BMC Genomics 13, 645.
Gosselin, D., Link, V. M., Romanoski, C. E., Fonseca, G. J., Eichenfield, D. Z., Spann, N. J., Stender, J. D., Chun, H. B., Garner, H., Geissmann, F., et al. (2014). Environment Drives Selection and Function of Enhancers Controlling Tissue-Specific Macrophage Identities. Cell 159, 1327-1340.
Hanna, R. N., Carlin, L. M., Hubbeling, H. G., Nackiewicz, D., Green, A. M., Punt, J. A., Geissmann, F., and Hedrick, C. C. (2011). The transcription factor NR4A1 (Nur77) controls bone marrow differentiation and the survival of Ly6C− monocytes. Nat. Immunol. 12, 778-785.
Hanna, R. N., Shaked, I., Hubbeling, H. G., Punt, J. A., Wu, R., Herrley, E., Zaugg, C., Pei, H., Geissmann, F., Ley, K., et al. (2012). NR4A1 (Nur77) deletion polarizes macrophages toward an inflammatory phenotype and increases atherosclerosis. Circ. Res. 110, 416-427.
Hanna, R. N., Cekic, C., Sag, D., Tacke, R., Thomas, G. D., Nowyhed, H., Herrley, E., Rasquinha, N., McArdle, S., Wu, R., et al. (2015). Patrolling monocytes control tumor metastasis to the lung. Science.
Heintzman, N. D., Stuart, R. K., Hon, G., Fu, Y., Ching, C. W., Hawkins, R. D., Barrera, L. O., Van Calcar, S., Qu, C., Ching, K. A., et al. (2007). Distinct and predictive chromatin signatures of transcriptional promoters and enhancers in the human genome. Nat. Genet. 39, 311-318.
Heinz, S., Benner, C., Spann, N., Bertolino, E., Lin, Y. C., Laslo, P., Cheng, J. X., Murre, C., Singh, H., and Glass, C. K. (2010). Simple combinations of lineage-determining transcription factors prime cis-regulatory elements required for macrophage and B cell identities. Mol. Cell 38, 576-589.
Heinz, S., Romanoski, C. E., Benner, C., Allison, K. A., Kaikkonen, M. U., Orozco, L. D., and Glass, C. K. (2013). Effect of natural genetic variation on enhancer selection and function. Nature.
Hettinger, J., Richards, D. M., Hansson, J., Barra, M. M., Joschko, A.-C., Krijgsveld, J., and Feuerer, M. (2013). Origin of monocytes and macrophages in a committed progenitor. Nat. Immunol. 14, 821-830.
Hnisz, D., Abraham, B. J., Lee, T. I., Lau, A., Saint-Andre, V., Sigova, A. A., Hoke, H A., and Young, R. A. (2013). Super-enhancers in the control of cell identity and disease. Cell 155, 934-947.
Hnisz, D., Schuijers, J., Lin, C. Y., Weintraub, A. S., Abraham, B. J., Lee, T. I., Bradner, J. E., and Young, R. A. (2015). Convergence of Developmental and Oncogenic Signaling Pathways at Transcriptional Super-Enhancers. Mol. Cell 58, 362-370.
Jacob, A., Zhou, M., Wu, R., Halpern, V. J., Ravikumar, T. S., and Wang, P. (2007). PRO-INFLAMMATORY CYTOKINES FROM KUPFFER CELLS DOWNREGULATE HEPATOCYTE EXPRESSION OF ADRENOMEDULLIN BINDING PROTEIN-1. Biochim. Biophys. Acta 1772, 766-772.
Kaikkonen, M. U., Spann, N. J., Heinz, S., Romanoski, C. E., Allison, K. A., Stender, J. D., Chun, H. B., Tough, D. F., Prinjha, R. K., Benner, C., et al. (2013). Remodeling of the Enhancer Landscape during Macrophage Activation Is Coupled to Enhancer Transcription. Mol. Cell 51, 310-325.
Kohyama, M., Ise, W., Edelson, B. T., Wilker, P. R., Hildner, K., Mejia, C., Frazier, W. A., Murphy, T. L., and Murphy, K. M. (2009). Role for Spi-C in the development of red pulp macrophages and splenic iron homeostasis. Nature 457, 318-321.
Lavin, Y., Winter, D., Blecher-Gonen, R., David, E., Keren-Shaul, H., Merad, M., Jung, S., and Amit, I. (2014). Tissue-Resident Macrophage Enhancer Landscapes Are Shaped by the Local Microenvironment. Cell 159, 1312-1326.
Li, L., Liu, Y., Chen, H., Li, F., Wu, J., Zhang, H., He, J., Xing, Y., Chen, Y., Wang, W., et al. (2015). Impeding the interaction between Nur77 and p38 reduces LPS-induced inflammation. Nat. Chem. Biol. 11, 339-346.
Liao, X., Sharma, N., Kapadia, F., Zhou, G., Lu, Y., Hong, H., Paruchuri, K., Mahabeleshwar, G. H., Dalmas, E., Venteclef, N., et al. (2011). Kruippel-like factor 4 regulates macrophage polarization. J. Clin. Invest 121, 2736-2749.
Mahabeleshwar, G. H., Kawanami, D., Sharma, N., Takami, Y., Zhou, G., Shi, H., Nayak, L., Jeyaraj, D., Grealy, R., White, M., et al. (2011). The myeloid transcription factor KLF2 regulates the host response to polymicrobial infection and endotoxic shock. Immunity 34, 715-728.
Muller, P. A., Koscsó, B., Rajani, G. M., Stevanovic, K., Berres, M.-L., Hashimoto, D., Mortha, A., Leboeuf, M., Li, X.-M., Mucida, D., et al. (2014). Crosstalk between muscularis macrophages and enteric neurons regulates gastrointestinal motility. Cell 158, 300-313.
Nakamura, A., Ebina-Shibuya, R., Itoh-Nakadai, A., Muto, A., Shima, H., Saigusa, D., Aoki, J., Ebina, M., Nukiwa, T., and Igarashi, K. (2013). Transcription repressor Bach2 is required for pulmonary surfactant homeostasis and alveolar macrophage function. J. Exp. Med. 210, 2191-2204.
Odegaard, J. I., and Chawla, A. (2011). Alternative macrophage activation and metabolism. Annu. Rev. Pathol. 6, 275-297.
Odegaard, J. I., Ricardo-Gonzalez, R. R., Goforth, M. H., Morel, C. R., Subramanian, V., Mukundan, L., Red Eagle, A., Vats, D., Brombacher, F., Ferrante, A. W., et al. (2007). Macrophage-specific PPARgamma controls alternative activation and improves insulin resistance. Nature 447, 1116-1120.
Okabe, Y., and Medzhitov, R. (2014). Tissue-Specific Signals Control Reversible Program of Localization and Functional Polarization of Macrophages. Cell 157, 832-844. Okabe, Y., and Medzhitov, R. (2016). Tissue biology perspective on macrophages. Nat. Immunol. 17, 9-17.
Ostuni, R., Piccolo, V., Barozzi, I., Polletti, S., Termanini, A., Bonifacio, S., Curina, A., Prosperini, E., Ghisletti, S., and Natoli, G. (2013). Latent Enhancers Activated by Stimulation in Differentiated Cells. Cell 152, 157-171.
Paolicelli, R. C., Bolasco, G., Pagani, F., Maggi, L., Scianni, M., Panzanelli, P., Giustetto, M., Ferreira, T. A., Guiducci, E., Dumas, L., et al. (2011). Synaptic pruning by microglia is necessary for normal brain development. Science 333, 1456-1458.
Parkhurst, C. N., Yang, G., Ninan, I., Savas, J. N., Yates III, J. R., Lafaille, J. J., Hempstead, B. L., Littman, D. R., and Gan, W.-B. (2013). Microglia Promote Learning-Dependent Synapse Formation through Brain-Derived Neurotrophic Factor. Cell 155, 1596-1609.
Pei, L., Castrillo, A., Chen, M., Hoffmann, A., and Tontonoz, P. (2005). Induction of NR4A orphan nuclear receptor expression in macrophages in response to inflammatory stimuli. J. Biol. Chem. 280, 29256-29262.
Perdiguero, E. G., and Geissmann, F. (2016). The development and maintenance of resident macrophages. Nat. Immunol. 17, 2-8.
Schmidl, C., Renner, K., Peter, K., Eder, R., Lassmann, T., Balwierz, P. J., Itoh, M., Nagao-Sato, S., Kawaji, H., Carninci, P., et al. (2014). Transcription and enhancer profiling in human monocyte subsets. Blood blood—2013-02-484188.
Schneider, C., Nobs, S. P., Kurrer, M., Rehrauer, H., Thiele, C., and Kopf, M. (2014). Induction of the nuclear receptor PPAR-y by the cytokine GM-CSF is critical for the differentiation of fetal monocytes into alveolar macrophages. Nat. Immunol. 15, 1026-1037.
Sica, A., and Mantovani, A. (2012). Macrophage plasticity and polarization: in vivo veritas. J. Clin. Invest. 122, 787-795.
Tacke, R., Hilgendorf, I., Garner, H., Waterborg, C., Park, K., Nowyhed, H., Hanna, R. N., Wu, R., Swirski, F. K., Geissmann, F., et al. (2015). The transcription factor NR4A1 is essential for the development of a novel macrophage subset in the thymus. Sci. Rep. 5, 10055.
Varol, C., Landsman, L., Fogg, D. K., Greenshtein, L., Gildor, B., Margalit, R., Kalchenko, V., Geissmann, F., and Jung, S. (2007). Monocytes give rise to mucosal, but not splenic, conventional dendritic cells. J. Exp. Med. 204, 171-180.
Wan, Y., Chong, L.-W., and Evans, R. M. (2007). PPAR-gamma regulates osteoclastogenesis in mice. Nat. Med. 13, 1496-1503.
Wang, H., Yang, H., Shivalila, C. S., Dawlaty, M. M., Cheng, A. W., Zhang, F., and Jaenisch, R. (2013). One-step generation of mice carrying mutations in multiple genes by CRISPR/Cas-mediated genome engineering. Cell 153, 910-918.
Whyte, W. A., Orlando, D. A., Hnisz, D., Abraham, B. J., Lin, C. Y., Kagey, M. H., Rahl, P. B., Lee, T. I., and Young, R. A. (2013). Master transcription factors and mediator establish super-enhancers at key cell identity genes. Cell 153, 307-319.
Yona, S., Kim, K.-W., Wolf, Y., Mildner, A., Varol, D., Breker, M., Strauss-Ayali, D., Viukov, S., Guilliams, M., Misharin, A., et al. (2012). Fate Mapping Reveals Origins and Dynamics of Monocytes and Tissue Macrophages under Homeostasis. Immunity.
Zheng, Y., Josefowicz, S., Chaudhry, A., Peng, X. P., Forbush, K., and Rudensky, A. Y. (2010). Role of conserved non-coding DNA elements in the Foxp3 gene in regulatory T-cell fate. Nature 463, 808-812.

### Example 2

### Supplemental Materials and Methods

### Cell Preparation and Isolation

Bone marrow monocytes were harvested from femurs, tibias and hip bones of 8-12 week old male C57BL/6 mice (strain 006664) obtained from the Jackson laboratory (Bar Harbor, Me.). Prior to FACS staining cells were subject to a brief (3 minute) red blood cell lysis (RBC lysis buffer, EBiosciences) at room temperature. All FACS staining was performed in FACS buffer (DPBS+10% FCS+2 mM EDTA). Prior to surface staining FC receptors were blocked with anti-CD16/32 (clone 93) for 30 minutes. Surface staining was performed for 30 minutes in a final volume of 500 μl for FACS sorts and 100 μl for regular flow cytometry. Surface staining was performed using Live/Dead Yellow (Thermo Fisher) and antibody combinations in accordance with the gating schemes in the figures. Lineage positive cells were identified using pooled APC conjugated anti-CD3, CD19, Ly6G and NK1.1 (clones145-2C11, 1D3, IA8 and PK136 respectively). Additional cell surface markers used were CD117-PE-Cy7 (ACK2), CD115 PE or BV421 (clone AFS98), Ly6C APC-Cy7 or PerCP-Cy5.5 (clone HK1.4), CD135-PE (clone A2F10.1), CD11c-FITC (clone N418), F4/80 PE-Cy7 (clone BM8), CD11b FITC (clone M1/70) and MHCII-BV605 (clone M1/70). All antibodies were obtained from Biolegend (San Diego, Calif.). Samples were washed twice in at least 200 μl FACS buffer before acquisition. Cells were sorted using a FACS Aria II (BD biosciences) and conventional flow cytometry using an LSRII (BD biosciences). All flow cytometry was performed on live cells.

### ChIP and Sequencing Library Preparation

ChIP assays for histone modifications were performed as previously described (Gilfillan et al., 2012). To obtain sufficient numbers of MDPs bone marrow from 10 mice were pooled and sorted. For each ChIP assay 500,000 FACS isolated cells were immediately washed with PBS and resuspended in MNase digestion buffer (50 mM Tris pH 8.0, 1 mM CaCl2, 0.2% Triton X-100). Cells were then digested in to mononucleosomal fragments using micrococcal nuclease (MNase, Affymetrix, CA). Enzymatic digestion was quenched by addition of 1/10 volume stop buffer (110 mM Tris pH 8.0, 55 mM EDTA), samples briefly sonicated using a Bioruptor (Diagenode, Belgium) and then adjusted to RIPA buffer conditions by adding an equal volume of 2×RIPA buffer (280 mM NaCl, 1.8% Triton x100, 0.2% SDS, 0.2% Sodium Deoxycholate, 5 mM EGTA). Immunoprecipitations were performed in a final volume of 100 μl. 2 μg of anti-H3K4me2 (Millipore 07-030) or 2 μg of anti-H3K27ac (Diagenode C15410196) were added and incubated overnight with rotation at 4° C. Antibody-antigen-DNA complexes were recovered by incubating for 2 hours with 10 pd Protein A Dynabeads previously washed in RIPA buffer (10 mM Tris pH 8.0, 140 mM NaCl, 1 mM EDTA, 1% Triton X-100, 0.2% SDS, 0.2% Sodium Deoxycholate). Complexes were then washed 5× in ice cold RIPA buffer and 1× in LiCl wash buffer (10 mM Tris pH 8.0, 250 mM LiCl, 1 mM EDTA, 0.5% Igepal CA-630, 0.5% Sodium deoxycholate), each wash was performed in 200 μl at 4° C. for 5 minutes with rotation. All above buffers were supplemented with 1× Protease inhibitor Cocktail, 1 mM PMSF and 5 mM Sodium Butyrate (Sigma). Beads were subject to a final wash in 200 μl ice cold TE buffer (Invitrogen) without protease inhibitors and eluted in 100 μl 1% SDS-TE buffer at 37° C. for 20 minutes. Finally, protein was treated with 2 μl proteinase K (Ambion) for 1h at 55° C. and DNA purified using a ChIP Clean & Concentrate column (Zymo, Irvine, Calif.) eluting in 30 μl final volume.

ChIP for PU.1 and C/EBPb were performed as previously described (Gosselin et al., 2014). 500,000 sorted cells were washed in PBS and immediately fixated for 9 minutes at room temperature in 1% methanol free formaldehyde in PBS (ThermoFisher Scientific). Fixation was quenched by addition of 1/20 volume 2.625M glycine solution and cells were washed twice with PBS. Cell pellets were then snap frozen in a dry ice/methanol bath and stored at −80° C. until needed. Nuclei were enriched by resuspending cell pellets in 10 mM HEPES pH 7.9, 85 mM KCl, 1 mM EDTA, 0.5% Igepal CA-630 and incubating on ice for 10 minutes. Nuclei were harvested by spinning at 3000 g for 10 minutes, and lysed in 130 μl lysis buffer (10 mM Tris pH 8.0, 100 mM NaCl, 1 mM EDTA, 0.5 mM EGTA, 0.1% Sodium Deoxycholate, 0.5% N-lauroylsarcosine). DNA was transferred in to Covaris micro tubes (Covaris, MA) sheared into 150-600 bp fragments using a Covaris E220 (14 mins, duty cycle 3%, 100 cycles per burst). Final volume was adjusted to 200 μl and 22 μl 10% Triton X-100 added, cell debris was then cleared by spinning at maximum speed for 5 minutes. 20 μl of protein A dynabeads pre-conjugated to 3 ug anti PU.1 or C/EBPb (sc-352× and sc-150× respectively, Santa Cruz Biotechnology, CA) were added to each chromatin preparation and incubated with rotation for 2h at 4° C. Antibody-antigen-DNA complexes were then washed in 3×WBI (20 mM Tris pH 7.4, 150 mM NaCl, 0.1% SDS, 1% Triton X-100, 2 mM EDTA,), 3× LiCl WB (10 mM Tris pH 7.4, 250 mM LiCl, 1% Triton X-100, 0.7% Sodium Deoxycholate, 1% Igepal CA-630) and 1×TET (TE, 0.2% Tween-20) buffer. All wash volumes were 200 μl. DNA was eluted in 1% SDS-TE for 30 minutes at 37° C., NaCl was added to a final concentration of 300 mM and crosslinking reversed overnight at 65° C. Finally, protein was treated with 2 μl proteinase K (Ambion) for 1 h at 55° C. and DNA purified using a ChIP Clean & Concentrate column (Zymo, Irvine, Calif.) eluting in 30 μl final volume.

ChIP-Seq libraries were prepared using an initial 0.3-5 ng DNA using the ThruPlex-FD kit (Rubicon Genomics, MI) in accordance with the manufacturer's guidelines. RNA-Seq libraries were prepared using the Tru-Seq v2 library preparation kit (Illumina, La Jolla, Calif.) in accordance with the manufacturer's instructions.

### RNA-Seq Differential Expression Analysis

Sequencing libraries were sequenced using an Illumina HiSeq at the LIAI sequencing core facility. RNA-Seq libraries were sequenced as paired-end 50 base reads, and ChIP-Seq libraries were sequenced as single-end 50 base reads. Illumina BCL files were converted into FASTQ format using bcl2fastq (v1.8.4, Illumina).

Paired end RNA-Seq reads were mapped to the mouse mm10 reference genome using RNA-STAR v2.3.0 (Dobin et al., 2013) compiled with gene models derived from the Ensembl v73 genome annotation set. Gene counts were quantitated with the same annotation set using featureCounts (Liao et al., 2013). Differential expression analysis was performed using edgeR v3.4.2 (Robinson et al., 2010). Variance calculations were computed using the common dispersion method and sequencing depth differences adjusted using the ‘relative log expression’ method. Differential expression was computed against all pairwise comparisons and an FDR (Benjamini-Hochberg) corrected P-value threshold of 1e-5 applied for calling statistical significance. RPKM calculations were computed in edgeR using gene lengths derived from featureCounts. Publicly available RNA-Seq data from Levin et al were obtained from GEO GSE63340 (Levin et al., 2014) and processed as above.

### ChIP-Seq Peak Calling, Differential Binding and UCSC Genome Browser Visualization

ChIP-Seq reads were mapped to the mm10 reference genome using Bowtie (v, Langmead et al., 2009). Homer (v4.4, Heinz et al., 2010) was used for ChIP-Seq peak calling.

Enhancer regions were defined by merging biological replicates for H3K4me2 libraries and running the findPeaks algorithm with ‘style-histone’ against the MNase-treated DNA input control. H3K4me2 profiles defined by each cell type were then aggregated using mergePeaks to give the final H3K4me2 peak set. These peaks were assigned to target genes using HOMER, and enhancers defined as H3K4me2 peaks whose centers were >2.5 kb from the nearest annotated transcription start site. Enhancer activity was measured by quantitating H3K4me2 and H3K27ac within these defined enhancer regions. Differences in sequencing depth were accounted for by normalizing to an effective library size of 1×10⁷ reads per lane. Transcription factor ChIP-Seq peaks were called using HOMER by running the findPeaks algorithm with the ‘style-factor’ flag using CH2O treated input DNA control as background sequence. Transcription factor peaks were called independently for each lane and merged using mergePeaks prior to quantification of tags within peaks using the annotatePeaks command in HOMER. Visualization was performed at the UCSC genome browser using bigWig files generated in HOMER.

ChIP-Seq data for Lavin et al (2014) were obtained using GEO accession GSE63339 and processed as above. Human ChIP-Seq data were accessed from GEO accession SRP015328 and mapped to the human hg19 reference genome using the protocols outlined above. The bigWig file of human monocyte DNase-Seq was downloaded from the ENCODE project at <URL: https://www.encodeproject.org/files/ENCFF000TAU/@@download/ENCFF000TAU.bigWig > accessed Sep. 10, 2015, and visualized directly in the UCSC genome browser.

### Microarray Data

Human monocyte Klf2 expression was obtained from NCBI GEO Profiles under accession GDS4219, data shown are for probe ID 219371_s_at, which is representative of the three probes available for Klf2 on this array type in this experiment.

### Differential Enhancer Profiling, Hierarchical Clustering and ChIP-Sea Visualization

Differential histone and transcription factor binding were determined using edgeR. To identify patterns of enhancer usage between MDP, cMoP, Ly6C^{hi} and Ly6C^{low} monocytes we employed a very permissive threshold for differential expression, this was to identify any weak signals in the data arising from these closely related cell types. Statistical significance was computed between all pairwise comparisons of cell types using an FDR corrected (Benjamini-Hochberg) p value threshold of 0.01 without the application of a fold-change cutoff. Variance was estimated using the common dispersion estimate by treating all conditions as pseudoreplicates of a single group. The final set of differentially enriched (DE) enhancers represents the union of all differentially bound enhancers determined by H3K4me2 or H3K27ac in any pairwise comparison.

In order to perform hierarchical clustering, matrices of tag counts for H3K4me2 and H3K27ac for the set of DE enhancers were obtained. Each matrix was independently mean centered and variance stabilized (with respect enhancers, row mean/row SD). The normalized matrices were then concatenated and subject to hierarchical clustering using a Euclidean distance and complete linkage clustering approach using the base functions in R. Based upon visual inspection, the results the tree was cut into nine clusters, the ordering of these clusters was used to order the final histogram figure (FIG. 9c). Within each cluster enhancers were then ranked according to their ‘peak score’ as determined by HOMER, this ranking was used to order rows within clusters in FIG. 9c. All PU.1 peaks overlapping each DE enhancer were extracted and H3K4me2/H3K27ac tag counts calculated +/−1 kb from each PU.1 peak center using HOMER's annotatePeaks with option ‘-ghist’. The final results were plotted using the R package gplots.

UCSC genome browser tracks were visualized using track hubs generated with the HOMER program makeBigWigHub.pl.

Differential PU.1 Peak Calling Between Ly6C^{hi} and Ly6C^{low} Mo

A complete monocyte PU.1 peak set was obtained by concatenating the PU.1 peaks defined in both Ly6C^{hi} and Ly6C^{low} Mo as described in ‘ChIP-Seq peak calling, differential binding and UCSC genome browser visualization’. Tag counts for PU.1 libraries were counted within this peak set for both monocyte populations and differential binding an analysis was performed in edgeR using the same procedures as for differential histone binding. For PU.1 peaks an FDR corrected (Benjamini-Hochberg) p-value cutoff of 1e-5 and log 2 fold change cutoff of 3 were applied.

### Enhancer Cloning

All specific cloning details for individual sequences are in Table 1. The Nr4a1 5′UTR and 650 bases of promoter sequence were cloned into the KpnI and SalI sites of a minimal pGL4.10 luc2 reporter vector. This vector, termed pGL4.10-TSS was used as the host vector for all enhancer clones. All enhancer sequences were isolated from genomic DNA by PCR using the primers detailed in Table 1. For PCR using Phusion High-fidelity polymerase (NEB, MA) 10 μl 5×HF buffer, 1 μl 10 mM dNTP, 2.5 μl F primer (10 uM), 2.5 μl R primer (10 uM) 2 μl mouse genomic DNA (250 ng), 1.5 μl DMSO, 30 μl H2O and 0.5 μl Phusion polymerase were used per reaction. PCR was performed using the following protocol at the indicated annealing temperature: −1×98° C. 30s; 30×98° C. 10s, anneal 30s, 72° C. 30s; 72° C. 10 min. For PCR using LA Taq GC buffer (Clontech, Mountain View, Calif.) 25 μl GC buffer I, 8 μl dNTP, 2.5 μl F primer (10 uM), 2.5 μl R primer (10 uM), 2 μl DNA (250 ng), 9.5 μl H2O and 0.5 μl LA-Taq were used per reaction. PCR was performed as per the manufacturer's instructions using the annealing temperatures in Table 1 and 1-minute extension time. PCR products were purified by gel extraction using Zymoclean Gen DNA recovery columns (Zymo, Irvine, Calif.). All cloning was performed at 1:4 vector. insert molar ratio using NEB reagents (Ipswich, Mass.) and clones were validated by PCR sequencing. PCR was performed using a MyCycler (BioRad, Irvine, Calif.)

### Transfection and Overexpression Experiments

RAW264.7 macrophages were obtained from ATCC and maintained at low passage (<15) in DMEM+(10% FCS, 1% Pen/Strep, 1% L-glut). Transfection studies were performed using 0.3 ug pGL4.10 luciferase reporter and 0.2 ug cDNA or 0.5 ug pGL4.10 luciferase reporter only, in addition 0.04 ug pmaxGFP (Lonza, Basel) and 0.01 ug pTK Renilla (ThermoFisher, Waltham, Mass.) luciferase control were added. 0.55 ug of plasmid DNA was complexed at 8:1 ratio with lipofectamine LTX as per manufacturer's guidelines and 100 μl per well given to RAW.264.7 cells at ˜70% confluency and activity measured typically 12-16 hours later using the Dual Luciferase Reporter Assay (Promega, Madison, Wis.) on Sirius luminometer (Titerkek-Berthold, Pforzheim, Germany).

shRNA Virus BMT Experiments

PLAT-E packaging cells were grown to 60-70% confluency in DMEM (Gibco, +10% FCS, 1% L-glut, 1 μg/ml puromycin, 10 μg/ml blasticidin) in 10 cm dishes at 37° C. in 10% CO2. Pools of three UltramiR (TRANSomiC technologies, Huntsville, Ala.) retroviral vectors (LMN) for Klf2 (ULTRA-3224750, ULTRA-3224748, ULTRA-3224747), Klf4 (ULTRA-3224770, ULTRA-3224769, ULTRA-3224768) and Non-targeting shRNA control were transfected into PLAT-E cells using JetPrime transfection in accordance with the manufacture's guidelines. 7 pg total DNA was transfected per 10 cm plate. 1 hour prior to transfection media was replaced with antibiotic free media, cells were incubated overnight. The following were replaced with fresh 10 ml DMEM, without antibiotics. The following morning the retroviral supernatant was harvested and used for transduction of bone marrow stem cells (below). 8 ml fresh DMEM was added to the cells and the last step repeated the following day.

Murine stem cells were isolated from bone marrow by negative selection using the EasySep Mouse Hematopoietic Progenitor Isolation Kit (StemCell technologies, Vancouver, BC) in accordance with manufacturer's guidelines. Cells were resuspended in 2.5 ml of STEMSPAN SFEM media (StemCell Technologies, Vancouver) containing 200 ng/ml rmSCF, 40 ng/ml rhIL-6 and 20 ng/ml rmIL-3 in 6 well plates previously coated for 2 hours with 2 ml of 25 μg/ml human fibronectin and washed once with PBS. Bone marrow stem cells from one mouse were split between 2 wells and an equal volume (1.25 ml) retroviral supernatant was added to each well. Cells were then spun at 850 g for 1 h at 37° C. and incubated for 22h at 37° C. in 10% CO₂. A second batch of retrovirus was added the following day and spin transduction repeated. Following the second transduction cells were harvested and transferred via retroorbital injection into lethally irradiated C57BU6 host mice (2×600 Rads, 2-3 hours apart). Bone marrow was allowed to reconstitute for 6 weeks prior to analysis.

### CRISPR Mouse Generation

sgRNA sequences were designed using the CRISPR Design web tool online at <URL: http://crispr.mit.edu>. To minimize off-target effects we only considered sgRNAs with a score >70. For each enhancer deletion two pairs of sgRNAs flanking the target region were designed that were at least 50 base pairs apart. sgRNA templates were ordered as Ultramers from IDT (Coralville, Iowa) as a chimeric T7 promoter sequence, variable crRNA (see Table 2 for individual sequence information) and invariable tracrRNA. In order to promote robust transcription from the T7 promoter the first two bases of each sgRNA sequence were substituted for guanines. dsDNA templates for each sgRNA were generated by PCR using the Pfu Ultra II polymerase (Agilent Technologies, Santa Clara, Calif.). PCR reactions were cleaned using ChIP Clean & Concentrate columns (Zymo, Irvine, Calif.) and used as input for in vitro transcription using the MEGAscript T7 kit (Thermo Fisher, Waltham, Mass.) in accordance with the manufacturer's instructions. RNA was cleaned using the MEGAclear kit (Thermo Fisher) in accordance with the manufacturer's guidelines.

For embryo injections 0.5 day fertilized embryos were collected from 3-4 week-old superovulated C57BL/6 females (Harlan, WI) by injecting 5.0 I.U each of PMSG (Sigma Aldrich) and hCG (Sigma Aldrich). Embryos were transferred into M2 medium (Millipore) and injected into the cytoplasm with sgRNA mixed at 25 ng/μl each along with 50 ng/μl Cas9 mRNA (GeneArt CRISPR Nuclease mRNA, Thermo Fisher) to give a final 150 ng/μl RNA in IDTE buffer. These injected embryos were cultured in an incubator in KSOMaa medium (Zenith) in a humidified atmosphere of 5% CO2 at 37 C over night. The embryos were implanted at 2-cell stage into recipient pseudo pregnant ICR female mice. Embryo injections were performed at the University of California, San Diego transgenic core facility.

### CRISPR Mouse Breeding and Genotyping

Founder mice were screened for the presence of one or two altered alleles using a PCR strategy that flanks the expected mutation. PCRs were designed such that the wild type product is 2-3 kb and the respective deletion allele around 1 kb shorter. Primer sequences and PCR details are in Table 3. PCRs were carried out using the specified kits in accordance with the manufacturer's guidelines, with primer extension and annealing temperatures stated in the table. Founder animals were crossed with C57BU6 mice obtained from the Jackson laboratory (strain 000664) to obtain heterozygous F1 animals that were interbred via sibling mating to generate Nr4a1se_2^{-/-}, Nr4a1se_6^{-/-}, and Nr4a1se_9^{-/-} mice. Observed phenotypes were present in homozygous null mice derived from at least three independent founder mice for each strain. For the Nr4a1se_2^{-/-} strain we also confirmed by Sanger sequencing that the downstream DNA sequence from the deletion into the Nr4a1 first intron was not modified

### LPS Challenge

Male mice aged 8-15 weeks old were intraperitoneally injected with 2.5*10⁶ EU/kg (equivalent to 2.5 mg/kg) Ultrapure LP-EB (Invivogen) in PBS. Mice were age and sex matched in all experiments. Prior to injection LPS was sonicated for 5 minutes at room temperature in a bath sonicator (FS20H, Fisher Scientific). Mice were monitored three times daily for the first 72 hours and twice daily thereafter.

### B16F10, Histology and Microscopy Quantification

300,000 B16F10 melanoma cells were intravenously injected by tail vein injection into recipient mice as previously described (Hanna et al 2015). 18 days following injection mice were sacrificed, lungs were filled with zinc buffered formalin and stored in the same buffer before embedding into paraffin blocks. Sections were cut at 4 μm, adhered to positively charged slides and dried over night. Sections were dewaxed with Slide Brite, rehydrated and stained with hematoxylin (Thermofisher), differentiated with acid alcohol, blued with Scott's water and stained with eosin (Thermofisher). Slides were scanned with ZEISS AxioScan Z1 slide scanner using 10×/0.3 NA or 20×/0.8 NA objective.

### Klf Mice

For Lys2^{Cre} Klf2^{flox/flox} and Lys2^{Cre} Klf4^{flox/flox} studies bone marrow (femur, tibia and hip bone) and Cre negative littermate controls were harvested, cleaned and shipped overnight on wet ice in BMM (RPMI, 1% Hepes, 1% Anti/Anti (Gibco), 1% BME (Gibco), 1% NEAA (Gibco), 1% Sodium Pyruvate (Gibco), 10% FCS). Bone marrow was harvested by scraping bones clean and immersing in 70% ethanol for 10 seconds before extracting marrow by centrifugation at 5,900 g for 15 seconds in a 1.5 ml Eppendorf tube. Bone marrow was immediately resuspended in room temperature sterile PBS and injected into lethally irradiated recipient mice (2×600 rads) by retroorbital injection at a ratio of 3:1 (recipient to donor). Mice were allowed to reconstitute for at least 6 weeks prior to sacrifice and analysis.

TABLE 1
Details for cloning Nr4a1 enhancer candidates into PGL4.10 luciferase reporter.

| | | | | Anneal | | |
|---|---|---|---|---|---|---|
| | Enh_F_primer_seq | Enh_R_primer_seq | PCR | temp | RE | Com |
| Plasmid | (SEQ. ID. NOs.: 1-13) | (SEQ. ID. NOs.: 14-26) | protocol | (° C.) | Digestion | cells |
| pGL4_Nr4a1_E12 | ATATGGATCCCAATGTTGGGT | ATATGTCGACAAATCGCTGTGG | Phusion | 72 | Bamh1 + | Top10 |
| | CTCTTTCTCAATTAGTTGC | TTTGAATGCCA | HF | | SalI | OneShot |
| pGL4_Nr4a1_E11 | ATATGGATCCGATTGATGTGG | ATATGTCGACGTGTGCACTACC | | | | |
| | GAGGCCAGGGTT | ATGTCCAGCATG | Phusion | 72 | Bamh1 + | NEB |
| | | | HF | | SalI | Stable |
| pGL4_Nr4a1_E10 | ATATGGATCCAACAAAAGCAA | ATATGTCGACAGGGAGAACCAA | Phusion | 72 | Bamh1 + | NEB |
| | AACACTGTTTCATTAGCGG | GCTACCCAGGA | HF | | SalI | Stable |
| pGL4_Nr4a1_E09 | ATATGGATCCCCTAGACTGGA | ATATGTCGACAGAAAGATTACC | LA Taq | 66 | Bamh1 + | Top10 |
| | GTTAATGACGGTCGTGA | CACAAATCAAAACCAGGGCT | GC 1 | | SalI | OneShot |
| pGL4_Nr4a1_E08 | ATATGGATCCGTATATGAGTA | ATATGTCGACGGACAGCTTAAA | Phusion | 70.4 | Bamh1 + | NEB |
| | CACTGTAGCTCTTTTCAGACA | GAGACAGGCTGAGAT | HF | | SalI | Stable |
| | CAC | | | | | |
| pGL4_Nr4a1_E07 | ATATGGATCCCCCAGAGCACG | ATATGTCGACCATCCTGTCCTG | LA Taq | 66 | Bamh1 + | Top10 |
| | GCTAAGGGGT | AGCAAGCCCTT | GC 1 | | SalI | OneShot |
| pGL4_Nr4a1_E06 | ATATGGATCCTTCATGAGACA | ATATGTCGACTGAGGGATTCAT | LA Taq | 60 | Bamh1 + | Top10 |
| | TTATACCATCTCACATCT | CCATGCAGA | GC 1 | | SalI | OneShot |
| pGL4_Nr4a1_E05 | ATATGGATCCGGCTCAGAAGA | ATATGTCGACGTTTGTTTGTTT | LA Taq | 66 | Bamh1 + | Top10 |
| | AAGACAGTGTACGGTG | TGTTTTTTCGAGACAGGGTTTC | GC 1 | | SalI | OneShot |
| | | T | | | | |
| pGL4_Nr4a1_E04 | ATATGGATCCGACAGGCGATG | ATATCTCGAGTCTGCAATCCCA | Phusion | 72 | Bamh1 + | NEB |
| | GGATAAGACACCTG | GTGTGTCAGGAGAC | HF | | XhoI | Stable |
| pGL4_Nr4a1_E03 | ATATGGATCCAGGGAGGCAGT | ATATGTCGACGTCTAGCTACCT | Phusion | 72 | Bamh1 + | Top10 |
| | GTGGGCTGAA | CCATGAAACTCTGCACC | HF | | SalI | OneShot |
| pGL4_Nr4a1_E02 | ATATGGATCCCATGGGACCTG | ATATGTCGACATTCTCCCTCCA | Phusion | 72 | Bamh1 + | Top10 |
| | GCCAGGTTTCA | TATATACATCTGTTCTATCGAC | HF | | SalI | OneShot |
| | | AG | | | | |
| pGL4_Nr4a1_E01 | ATATGGATCCGGCTGGCAGCA | ATATGTCGACCGACCAGGAGGA | Phusion | 72 | Bamh1 + | Top10 |
| | GAAATCGGGAA | GGGGGTGTT | HF | | SalI | OneShot |
| pGL4_Nr4a1_TSS | ATATGGTACCGAAGGCCAGAG | ATATGAGCTCTCCCACTCCCTG | Phusion | 68.5 | KpnI + | Top10 |
| | TGCCTGTCC | TGGCCG | HF | | SacI | OneShot |

TABLE 2
crRNA design and sequences.

| | | | | crRNA sequence (including |
|---|---|---|---|---|
| | | | | G substitution for T7 |
| | Enhancer | | Guide sequence (genomic) | promoter) (SEQ. ID. NOs.: |
| sgRNA name | region | Score | (SEQ. ID. NOs.: 27-42) | 43-58) |
| E02_US_1 | E02 | 80 | GTGAACTGAACTCCCCACCG | GTGAACTGAACTCCCCACCG |
| E02_US_2 | E02 | 79 | GCGCTGAGATATATGAATGC | GCGCTGAGATATATGAATGC |
| E02_DS_1 | E02 | 82 | GGGCGGGGCGGTTCCTGATT | GGGCGGGGCGGTTCCTGATT |
| E02_DS_2 | E02 | 78 | GCAGCAGGGTCAGCGTGAAC | GCAGCAGGGTCAGCGTGAAC |
| E06_US_1 | E06 | 88 | TGCTTAGGCACGGTAGTCAT | GGCTTAGGCACGGTAGTCAT |
| E06_US_2 | E06 | 75 | TCTGGTCTGGTCACTACAAA | GCTGGTCTGGTCACTACAAA |
| E06_DS_1 | E06 | 83 | GTGATCTAACACACCCCCCT | GTGATCTAACACACCCCCCT |
| E06_DS_2 | E06 | 80 | GGGTTTGGGGCTAGTGTAAT | GGGTTTGGGGCTAGTGTAAT |
| E09_US_1 | E09 | 73 | GGGGTTTGACCTGAGCCATC | GGGGTTTGACCTGAGCCATC |
| E09_US_2 | E09 | 72 | GAGCTTTTGGTGTCTTGACC | GAGCTTTTGGTGTCTTGACC |
| E09_DS_1 | E09 | 73 | GGAGGGGTTAACTAACCCAC | GGAGGGGTTAACTAACCCAC |
| E09_DS_2 | E09 | 72 | GGATCAATAACTACTTGGCT | GGATCAATAACTACTTGGCT |
| E04_07_US_1 | E04_E07 | 77 | TAGCCATCTCCCAGTCAAGC | GAGCCATCTCCCAGTCAAGC |
| E04_07_US_2 | E04_E07 | 76 | ATGGACCCTTACTCCCAAAT | GTGGACCCTTACTCCCAAAT |
| E04_07_DS_1 | E04_E07 | 94 | GAGGTGAAGGGTCCCAATCG | GAGGTGAAGGGTCCCAATCG |
| E04_07_DS_2 | E04_E07 | 77 | CTGCGTTTTAAGCCTTATAA | GTGCGTTTTAAGCCTTATAA |

sgRNAs were ordered as ultramers composed of an upstream T7 promoter (underlined), crRNA (lower case) and invariant downstream tracrRNA sequence (boxed) as shown:

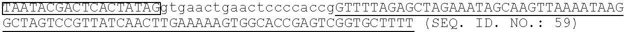

TABLE 3
PCR details for enhancer knockout mouse genotyping.

| | F primer | R primer | | | | WT | KO |
|---|---|---|---|---|---|---|---|
| | (SEQ. ID. | (SEQ. ID. | Polymerase | anneal | extend | product | product |
| Mouse | NOs.: 60-62) | NOs.: 63-65) | kit | (° C.) | (min) | size | size |
| Nr4a1se_9 | GCATCTCTGCTCCCCACTTT | CAGTAAGCCACCTTGAGCCA | NBE Phusion | 68 | 3 | 2.5 kb | 1 kb |
| | | | HF | | | | |
| Nr4a1e_6 | GGCTCCCAGTGTGACCTTTT | CCTGAACGCCTGAGCTAACA | LA Taq GCI | 58 | 2 | 1.8 kb | 500 bp |
| Nr4a1se_2 | CTGAGGCTCCTTATCGGGGA | CTGAATGCCCAAAACGCACC | NBE Phusion | 68 | 3 | 2.6 kb | 1 kb |
| | | | HF | | | | |

What is claimed is:

1. A method of modulating CD14^{dim}CD16⁺ (CD115⁺CD11b⁺GR1⁻ (Ly6C−)) monocytes or macrophages, the method comprising modulating expression or activity of Nr4a1 (Nur77).

2. A method of modulating CD14^{dim}CD16⁺ (CD115⁺CD11b⁺GR1⁻ (Ly6C−)) monocytes or macrophages, the method comprising modulating expression or activity of a transcription factor that regulates expression of Nr4a1 (Nur77).

3. A method of stimulating, promoting, increasing or inducing CD14^{dim}CD16⁺ monocyte or macrophage cell production, development, survival, proliferation, differentiation or activity, comprising modulating expression or activity of a transcription factor that regulates expression of Nr4a1 (Nur77).

4. The method of claim 2 or 3, wherein the transcription factor binds to the DNA sequence in the region Nr4a1se_2 in Ly6C− monocyte or upstream progenitor cell types thereof or a homologous region in CD14^{dim}CD16⁺ monocytes or upstream progenitor cell types thereof.

5. The method of any one of claims 2 to 4, wherein the transcription factor is Klf2, Klf4 or Nf-κB.

6. The method of any one of claims 1 to 5, wherein the method comprises inhibiting growth of a hyperproliferative cell, tumor cell, cancer cell, neoplastic cell, metastatic cell or tumor.

7. The method of any one of claims 2 to 6, wherein the method comprises administering an agonist or antagonist of the transcription factor.

8. The method of claim 7, wherein the method comprises administering an agonist of the transcription factor.

9. The method of claim 7 or 8, wherein the agonist or antagonist is an antibody, a small molecule, a peptide, an inhibitory nucleic acid, an allosteric inhibitor or a ligand mimetic.

10. The method of claim 9, wherein the antagonist is an antibody, or binding fragment thereof, that specifically binds to a Klf2 or Klf4 polypeptide.

11. The method of claim 9, wherein the antagonist is an inhibitory nucleic acid that targets or binds to an mRNA directing the expression of a Klf2 or Klf4 polypeptide.

12. The method of any one of claims 7 to 11, comprising administering the agonist or antagonist to a subject.

13. The method of claim 12, wherein the method comprises decreasing, reducing, inhibiting, suppressing, limiting or controlling an undesirable or aberrant immune response, immune disorder, inflammatory response, inflammation autoimmune response, autoimmune disease, adverse cardiovascular event or cardiovascular disease in the subject.

14. The method of claim 12, wherein the method comprises treating cancer in the subject.

15. A pharmaceutical composition comprising an agonist or antagonist of a transcription factor that regulates expression of Nr4a1 (Nur77) for treatment of an aberrant immune response, immune disorder, inflammatory response, inflammation, an autoimmune response, disorder or disease, cancer or an adverse cardiovascular event or cardiovascular disease in a subject.

16. The pharmaceutical composition of claim 15, wherein the transcription factor binds to the DNA sequence in the region Nr4a1se_2 in Ly6C− monocyte or upstream progenitor cell types thereof or a homologous region in CD14^{dim}CD16⁺ monocytes or upstream progenitor cell types thereof.

17. The pharmaceutical composition of claim 15 or 16, wherein the transcription factor is Klf2 or Klf4.

18. The pharmaceutical composition of any one of claims 15 to 17, wherein the agonist or antagonist is an antibody, a small molecule, a peptide, an inhibitory nucleic acid, an allosteric inhibitor or a ligand mimetic.

19. A method of identifying an agent that modulates, increases or decreases CD14^{dim}CD16⁺ (CD115⁺CD11b⁺GR1⁻ (Ly6C⁻)) monocyte or macrophage amounts or activity, the method comprising:
(a) contacting an experimental agent with an Nr4a1 promoter or enhancer region in the presence of Klf2, Klf4 and/or Nf-κB, wherein the Nr4a1 promote region is operably linked to a reporter nucleic acid;
(b) determining an amount of the reporter nucleic acid, or an amount of a transcribed or translated product thereof;
(c) comparing the amount determined in (b) to a control amount of the reporter nucleic acid, or the transcribed or translated product thereof, wherein a difference between the amount determined in (b) and a control amount indicates the experimental agent is an agent that modulates, increases or decreases CD14^{dim}CD16⁺ (CD115⁺CD11b⁺GR1⁻ (Ly6C⁻)) monocyte or macrophage amounts or activity.

20. The method of claim 19, wherein the experimental agent is an antibody or inhibitory nucleic acid.

21. The method of claim 19, wherein the antibody specifically binds to Klf2 or Klf4.

22. A method of identifying CD14^{dim}CD16⁺ (CD115⁺CD11b⁺GR1⁻ (Ly6C⁻)) monocyte functions through the use of homozygous or heterozygous mice deficient in Nr4a1se_2 sequence, or any vertebrates deficient or otherwise modified at a DNA sequence homologous to Nr4a1se_2.
